(19) United States
(12) Patent Application Publication
(10) Pub. No.: US 2025/0230153 A1
(43) Pub. Date: Jul. 17, 2025

(54) 2,3-DIHYDROBENZO[B][1,4]DIOXIN-6-YL CONTAINING COMPOUNDS USEFUL AS IMMUNOMODULATORS

(71) Applicant: BRISTOL-MYERS SQUIBB COMPANY, Princeton, NJ (US)

(72) Inventors: Timothy P. Connolly, Kenosha, WI (US); Piyasena Hewawasam, Middletown, CT (US); Juliang Zhu, Edison, NJ (US); Denis R. St. Laurent, Newington, CT (US); Kap-Sun Yeung, Madison, CT (US); Paul Michael Scola, Glastonbury, CT (US)

(21) Appl. No.: 18/402,803

(22) PCT Filed: Jun. 29, 2022
(86) PCT No.: PCT/US2022/073244

§ 371 Date: Jan. 3, 2024

Related U.S. Application Data
(60) Provisional application No. 63/218,555, filed on Jul. 6, 2021.

(51) Int. Cl. C07D 417/14; A61K 31/357; A61K 31/4025; A61K 31/4155; A61K 31/427; A61K 31/4433; A61K 31/4439; A61K 31/453; A61K 31/454; A61K 31/4545; A61K 31/497; A61K 31/5377; A61K 31/541; C07D 319/18; C07D 405/12; C07D 405/14; C07D 407/12; C07D 413/12; C07D 413/14; C07D 417/12

(52) U.S. Cl. CPC C07D 417/14; A61K 31/357; A61K 31/4025; A61K 31/4155; A61K 31/427; A61K 31/4433; A61K 31/4439; A61K 31/453; A61K 31/454; A61K 31/4545; A61K 31/497; A61K 31/5377; A61K 31/541; C07D 319/18; C07D 405/12; C07D 405/14; C07D 407/12; C07D 413/12; C07D 413/14; C07D 417/12

(57) ABSTRACT

The present disclosure generally relates to 2,3-dihydrobenzo[b][1,4]dioxin-6-yl containing compounds useful as immunomodulators. Provided herein are compounds, compositions comprising such compounds, and methods of their use. The disclosure further pertains to pharmaceutical compositions comprising at least one compound according to the disclosure that are useful for the treatment of various diseases, including cancer and infectious diseases.

### CROSS REFERENCE TO RELATED APPLICATION

This application claims the priority of U.S. Provisional Application Ser. No. 63/218,555 filed Jul. 6, 2021 which is herein incorporated by reference.

The present disclosure generally relates to 2,3-dihydrobenzo[b][1,4]dioxin-6-yl containing compounds useful as inhibitors of the PD-1/PD-L1 protein/protein and CD80/PD-L1 protein/protein interactions. Provided herein are compounds, compositions comprising such compounds, and methods of their use. The disclosure further pertains to pharmaceutical compositions comprising at least one compound according to the disclosure that are useful for the treatment of various diseases, including cancer and infectious diseases.

Programmed death-1 (CD279) is a receptor on T cells that has been shown to suppress activating signals from the T cell receptor when bound by either of its ligands, Programmed death-ligand 1 (PD-L1, CD274, B7-H1) or PD-L2 (CD273, B7-DC) (Sharpe et al., Nat. Imm. 2007). When PD-1 expressing T cells contact cells expressing its ligands, functional activities in response to antigenic stimuli, including proliferation, cytokine secretion, and cytolytic activity are reduced. PD-1/PD-Ligand interactions down regulate immune responses during resolution of an infection or tumor, or during the development of self tolerance (Keir Me, Butte M J, Freeman G J, et al. Annu. Rev. Immunol. 2008; 26: Epub). Chronic antigen stimulation, such as that which occurs during tumor disease or chronic infections, results in T cells that express elevated levels of PD-1 and are dysfunctional with respect to activity towards the chronic antigen (reviewed in Kim and Ahmed, Curr Opin Imm, 2010). This is termed “T cell exhaustion”. B cells also display PD-1/PD-ligand suppression and “exhaustion”.

PD-L1 has also been shown to interact with CD80 (Butte M J et al., Immunity 27:111-122 (2007)). The interaction of PD-L1/CD80 on expressing immune cells has been shown to be an inhibitory one. Blockade of this interaction has been shown to abrogate this inhibitory interaction (Paterson A M, et al., J Immunol., 187:1097-1105 (2011); Yang J, et al. J Immunol. August 1; 187(3):1113-9 (2011)).

Blockade of the PD-1/PD-L1 interaction using antibodies to PD-L1 has been shown to restore and augment T cell activation in many systems. Patients with advanced cancer benefit from therapy with a monoclonal antibody to PD-L1 (Brahmer et al., New Engl J Med 2012). Preclinical animal models of tumors have shown that blockade of the PD-1/PD-L1 pathway by monoclonal antibodies can enhance the immune response and result in the immune response to a number of histologically distinct tumors (Dong H, Chen L. J Mol Med. 2003; 81(5):281-287; Dong H, Strome S E, Salamoa D R, et al. Nat Med. 2002; 8(8):793-800).

Interference with the PD-1/PD-L1 interaction has also shown enhanced T cell activity in chronic infection systems. Chronic lymphocytic chorio meningitis virus infection of mice also exhibits improved virus clearance and restored immunity with blockade of PD-L1 (Barber D L, Wherry E J, Masopust D, et al. Nature 2006; 439(7077):682-687). Humanized mice infected with HIV-1 show enhanced protection against viremia and reduced viral depletion of CD4+ T cells (Palmer et al., J. Immunol 2013). Blockade of PD-1/PD-L1 through monoclonal antibodies to PD-L1 can restore in vitro antigen-specific functionality to T cells from HIV patients (Day, Nature 2006; Petrovas, J Exp. Med. 2006; Trautman, Nature Med. 2006; D'Souza, J. Immunol. 2007; Zhang, Blood 2007; Kaufmann, Nature Imm. 2007; Kasu, J. Immunol. 2010; Porichis, Blood 2011), HCV patients [Golden-Mason, J. Virol. 2007; Jeung, J. Leuk. Biol. 2007; Urbani, J. Hepatol. 2008; Nakamoto, PLoS Path. 2009; Nakamoto, Gastroenterology 2008] or HBV patients (Boni, J. Virol. 2007; Fisicaro, Gastro. 2010; Fisicaro et al., Gastroenterology, 2012; Boni et al., Gastro., 2012; Penna et al., J Hep, 2012; Raziorrough, Hepatology 2009; Liang, World J Gastro. 2010; Zhang, Gastro. 2008).

Blockade of the PD-L1/CD80 interaction has also been shown to stimulate immunity (Yang J., et al., J Immunol. August 1; 187(3):1113-9 (2011)). The immune stimulation resulting from blockade of the PD-L1/CD80 interaction has been shown to be enhanced through combination with blockade of further PD-1/PD-L1 or PD-1/PD-L2 interactions.

Alterations in immune cell phenotypes are hypothesized to be an important factor in septic shock (Hotchkiss, et al., Nat Rev Immunol (2013)). These include increased levels of PD-1 and PD-L1 and T ceoll apoptosis (Guignant, et al, Crit. Care (2011)). Antibodies directed to PD-L1 can reduce the level of Immune cell apoptosis (Zhang et al, Crit. Care (2011)). Furthermore, mice lacking PD-1 expression are more resistant to septic shock symptoms than wildtype mice (Yang J., et al., J Immunol. August 1; 187(3):1113-9 (2011)). Studies have revealed that blockade of the interactions of PD-L1 using antibodies can suppress inappropriate immune responses and ameliorate disease symptoms.

In addition to enhancing immunologic responses to chronic antigens, blockade of the PD-1/PD-L1 pathway has also been shown to enhance responses to vaccination, including therapeutic vaccination in the context of chronic infection (S. J. Ha, S. N. Mueller, E. J. Wherry et al., The Journal of Experimental Medicine, vol. 205, no. 3, pp. 543-555, 2008.; A. C. Finnefrock, A. Tang, F. Li et al., The Journal of Immunology, vol. 182, no. 2, pp. 980-987, 2009; M. -Y. Song, S.-H. Park, H. J. Nam, D. -H. Choi, and Y.-C. Sung, The Journal of Immunotherapy, vol. 34, no. 3, pp. 297-306, 2011).

The PD-1 pathway is a key inhibitory molecule in T cell exhaustion that arises from chronic antigen stimulation during chronic infections and tumor disease. Blockade of the PD-1/PD-L1 interaction through targeting the PD-L1 protein has been shown to restore antigen-specific T cell immune functions in vitro and in vivo, including enhanced responses to vaccination in the setting of tumor or chronic infection. Accordingly, agents that block the interaction of PD-L1 with either PD-1 or CD80 are desired.

Applicants found potent compounds that have activity as inhibitors of the interaction of PD-L1 with PD-1 and CD80, and thus may be useful for therapeutic administration to enhance immunity in cancer or infections, including therapeutic vaccine. These compounds are provided to be useful as pharmaceuticals with desirable stability, bioavailability, therapeutic index, and toxicity values that are important to their drugability.

In a first aspect, the present disclosure provides a compound of Formula (I):

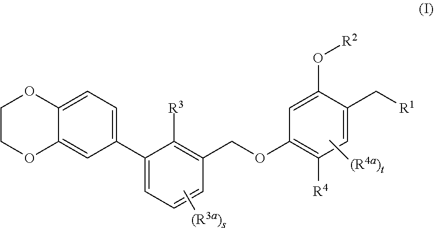

or a pharmaceutically acceptable salt thereof, wherein:
R¹ is independently

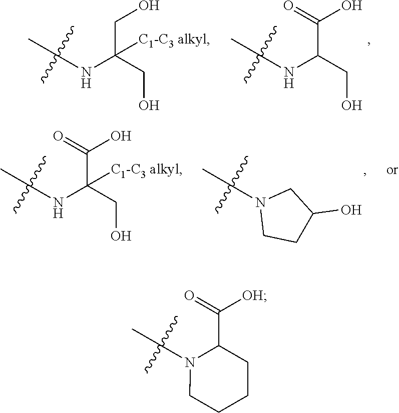

R² is independently —CH₂R^{2a}, —CH₂R^{2b}, or —(CH₂)ₘ(CHR^{a})ₙ—(CH₂)ₘR^{2b};
R^{2a} is independently C₄-C₆ cycloalkyl,

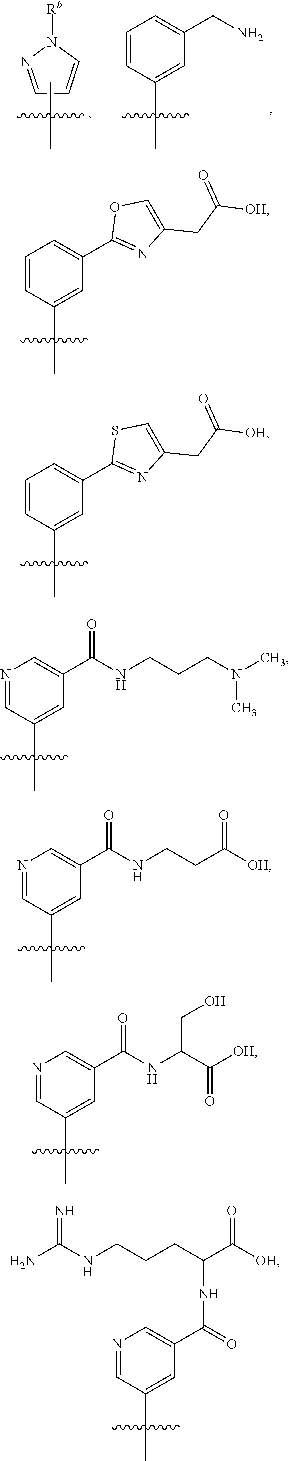

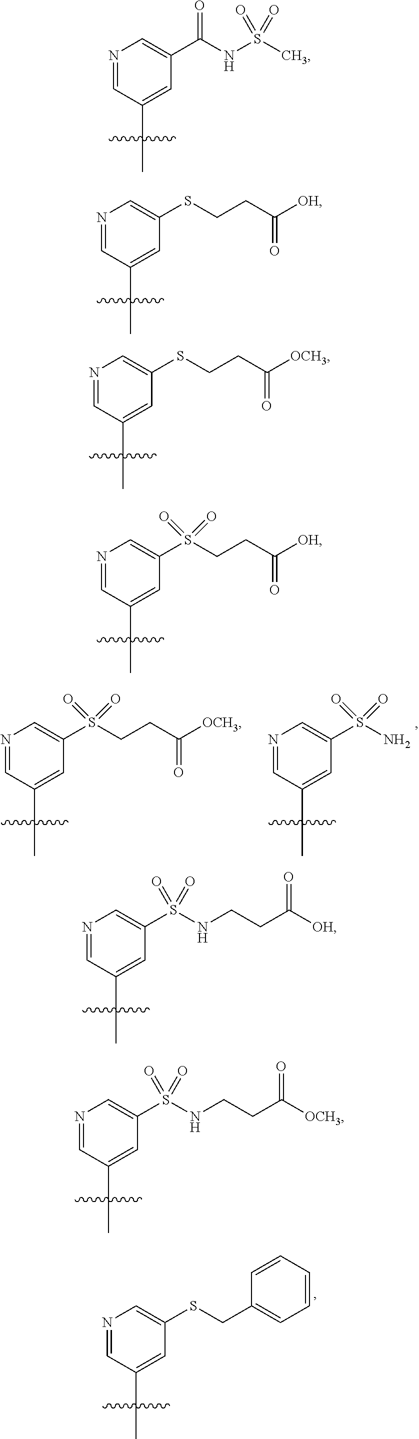

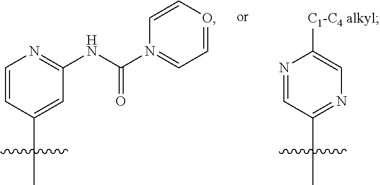

R^{2b} is independently OH, C₁-C₄ haloalkyl, —N(C₁-C₄ alkyl)₂, a 5- to 6-membered 5 heterocyclic ring selected from pyrrolidinyl, tetrahydropyranyl, piperidinyl, morpholinyl

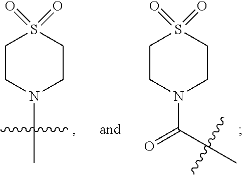 [SMILES: *N1CCS(=O)(=O)CC1 ; *C(=O)N1CCS(=O)(=O)CC1]

wherein said heterocyclic ring is substituted with 0 to 3 R^{c};
R³ and R⁴ are, at each occurrence, independently hydrogen, halogen, CN, C₁-C₄ alkyl, C₁-C₄haloalkyl, C₁-C₄ alkoxy, or C₁-C₄haloalkoxy;
R³ and R^{4a} are, at each occurrence, independently halogen, CN, C₁-C₄ alkyl,
C₁-C₄ haloalkyl, C₁-C₄ alkoxy, or C₁-C₄haloalkoxy;
R^{a} is independently H or C₁-C₃ alkyl;
R^{b} is independently C₁-C₄ alkyl substituted with 0 to 1 R^{d}, or pyridyl;
R^{c} is independently OH, CN, halogen, C₁-C₄ alkyl, C₁-C₄haloalkyl, —C(═O)OH, or —C(═O)(OC₁-C₄ alkyl);
R^{d} is independently OH, CN, halogen, C₁-C₄haloalkyl, —C(═O)OH, or —C(═O)(OC₁-C₄ alkyl);
m is independently 1, 2 or 3;
n is independently 0 or 1;
s and t are each independently 0, 1, or 2.

In a second aspect, within the scope of the first aspect, wherein:
R³ and R⁴ are, at each occurrence, independently hydrogen, halogen, CN, C₁-C₄ alkyl or C₁-C₄ haloalkyl;
R^{3a} and R^{4a} are, at each occurrence, independently halogen, CN, C₁-C₄ alkyl, or C₁-C₄ haloalkyl;
R^{c} is independently OH, F, C₁, C₁-C₃ alkyl, C₁-C₂haloalkyl, —C(═O)OH, or —C(═O)(OC₁-C₂ alkyl);
R^{d} is independently CN, —C(═O)OH, or —C(═O)(OC₁-C₄ alkyl);
m is independently 1 or 2;
n is independently 0 or 1;
s and t are each independently 0 or 1.

In a third aspect, the present disclosure provides a compound of Formula (II):

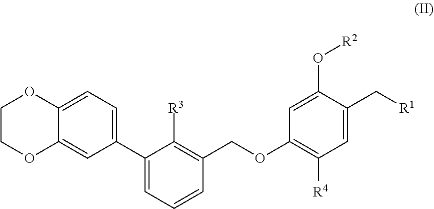

or a pharmaceutically acceptable salt thereof, wherein:
R¹ is independently

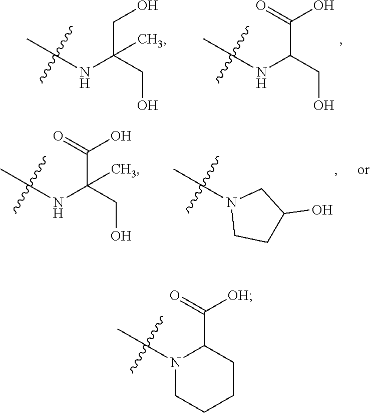

R² is independently —CH₂R^{2a}, —CH₂R^{2b}, or —(CH₂)ₘ(CHR^{a})ₙ—(CH₂)ₘR^{2b};
R^{2a} is independently cyclobutyl, cyclohexyl,

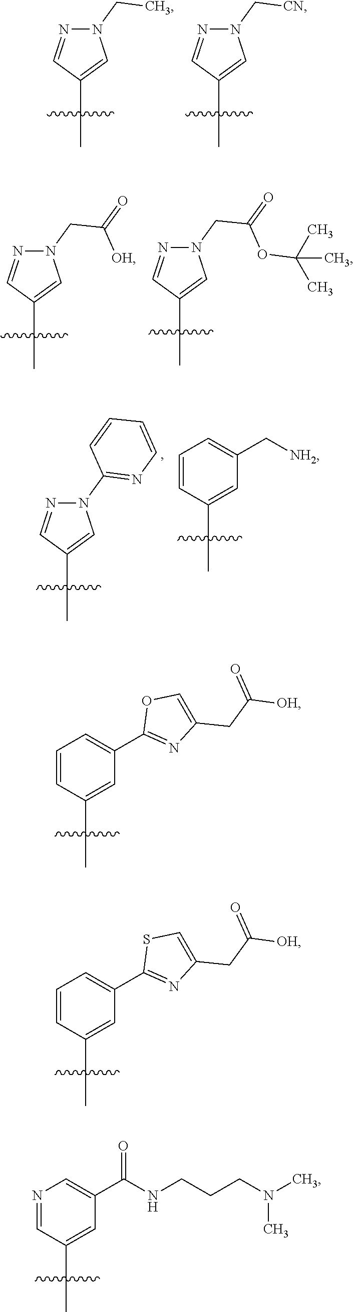

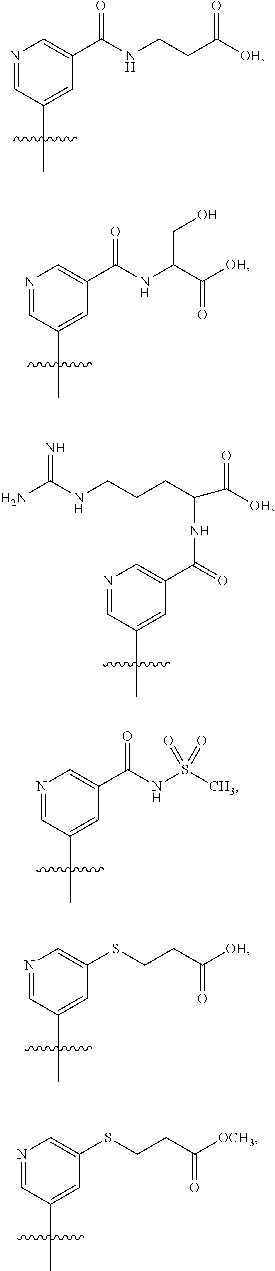 [SMILES: *c1cncc(C(=O)NCCC(=O)O)c1 ; *c1cncc(C(=O)NC(CO)C(=O)O)c1 ; *c1cncc(C(=O)NC(CCCNC(=N)N)C(=O)O)c1 ; *c1cncc(C(=O)NS(C)(=O)=O)c1 ; *c1cncc(SCCC(=O)O)c1 ; *c1cncc(SCCC(=O)OC)c1]

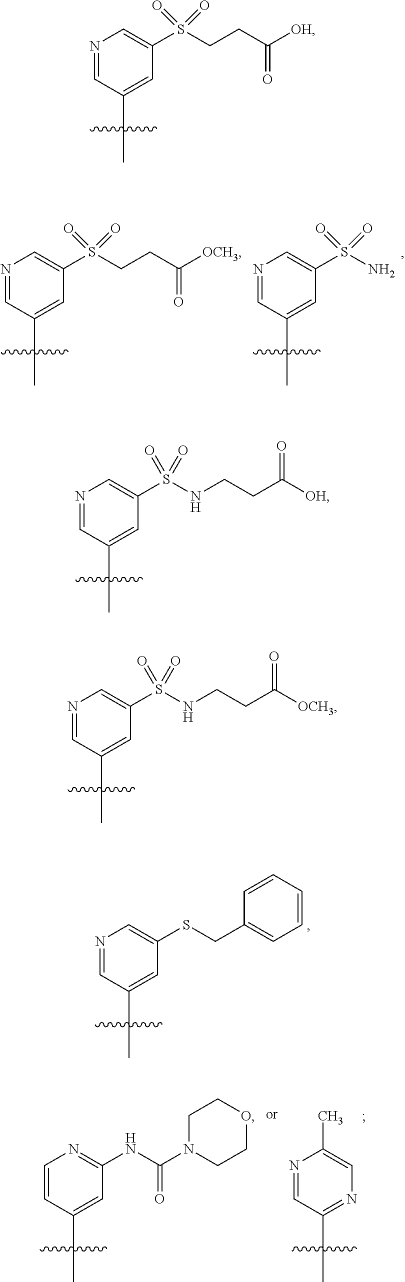

R^{2b} is independently OH, CF₃, —N(CH₃)₂,

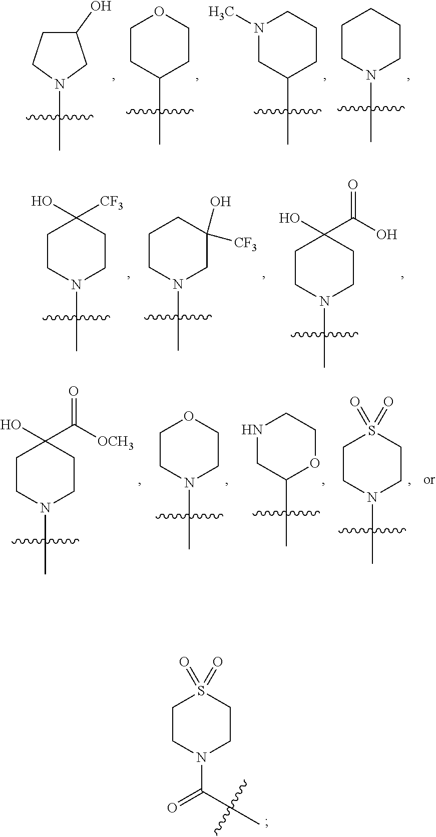

R³ is independently hydrogen, C₁-C₃ alkyl or halogen;
R⁴ is independently hydrogen, C₁-C₃ alkyl or halogen;
R^{a} is independently H or CH₃;
m is independently 1 or 2; and
n is independently 0 or 1.

In a fourth aspect, within the scope of the third aspect, wherein:
R² is independently —CH₂R^{2a};
R^{2a} is independently cyclobutyl, cyclohexyl,

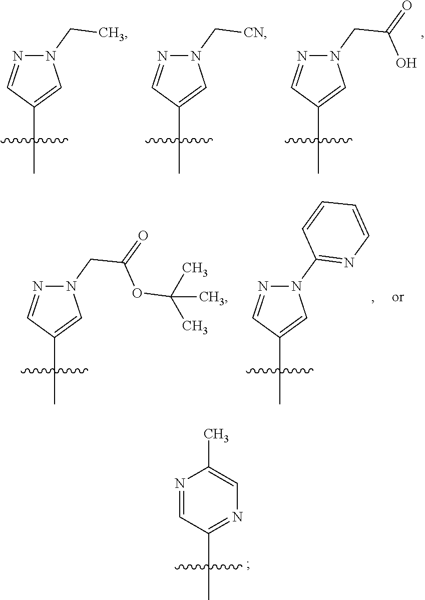

R³ is CH₃; and
R⁴ is Cl.

In a fifth aspect, within the scope of the third aspect, wherein:
R² is independently —CH₂R^{2a};
R^{2a} is independently

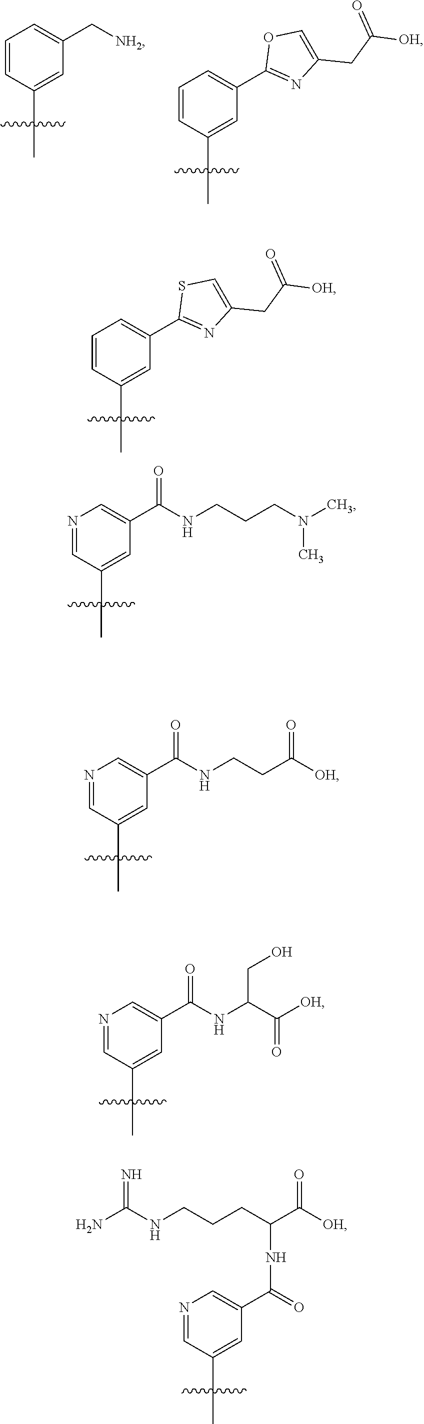

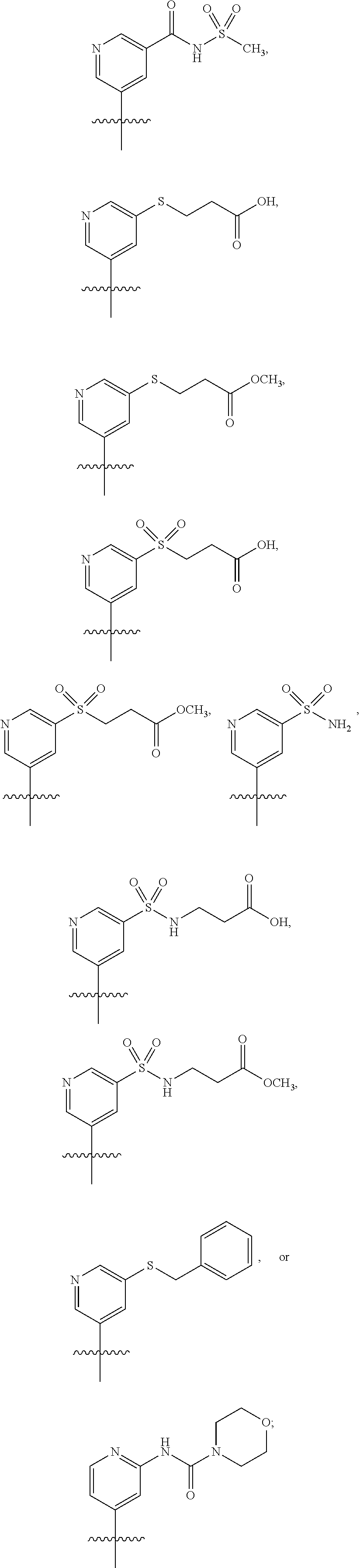

R³ is CH₃; and
R⁴ is Cl.

In a sixth aspect, within the scope of the third aspect, wherein:
R² is independently —CH₂R^{2b}, or —(CH₂)ₘ(CHR^{a})ₙ—(CH₂)ₘR^{2b};
R^{2b} is independently OH, CF₃, —N(CH₃)₂,

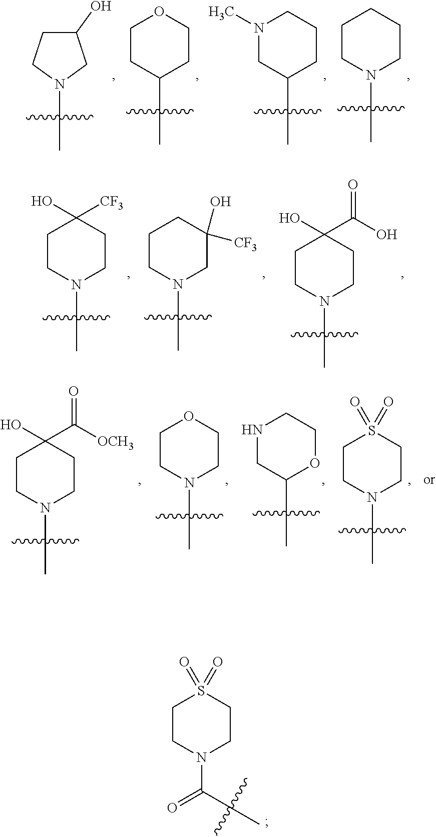

R³ is CH₃; and
R⁴ is Cl.

In one embodiment, R¹ is

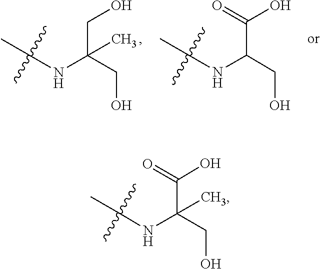

In another embodiment, R¹ is

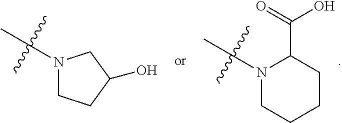 [SMILES: *N1CCC(O)C1 ; *N1CCCCC1C(=O)O]

In one embodiment, R¹ is

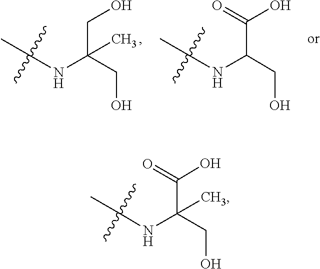

In another embodiment, R¹ is

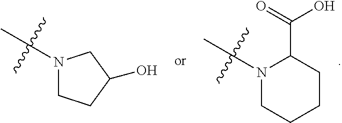 [SMILES: *N1CCC(O)C1 ; *N1CCCCC1C(=O)O]

In one embodiment, R² is —CH₂R^{2a}. In another embodiment, R² is —CH₂R^{2b} or —(CH₂)ₘ(CHR^{a})ₙ—(CH₂)ₘR^{2b}.

In one embodiment, R^{2a} is

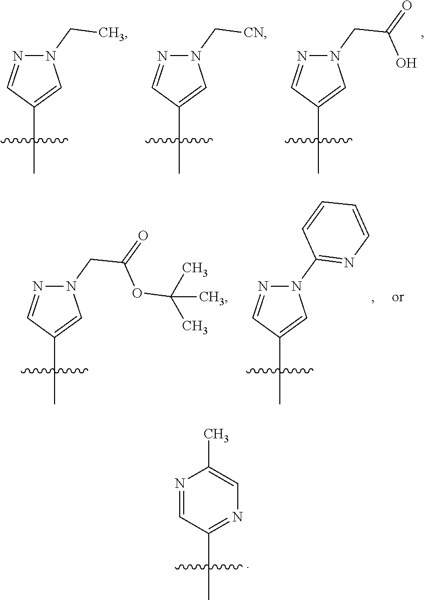

In another embodiment, R^{2a} is cyclobutyl, cyclohexyl,

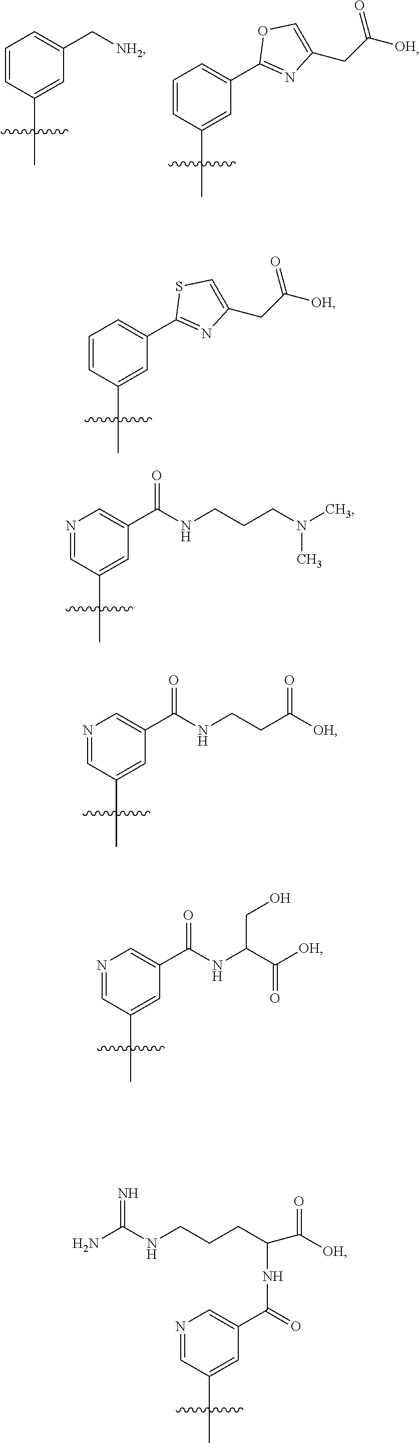

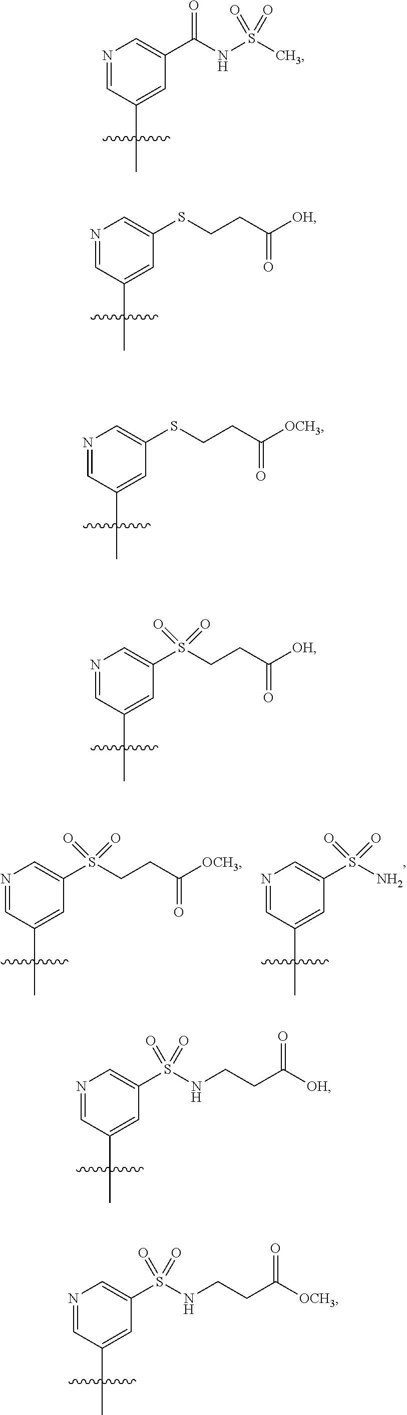

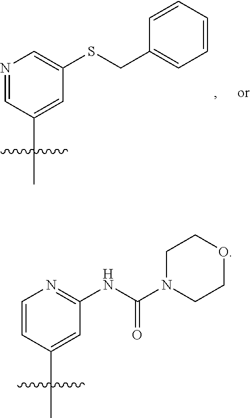 [SMILES: *c1cncc(SCc2ccccc2)c1 ; *c1ccnc(NC(=O)N2CCOCC2)c1]

In one embodiment, R^{2b} is OH, CF₃, or —N(CH₃)₂. In another embodiment, R^{2b} is

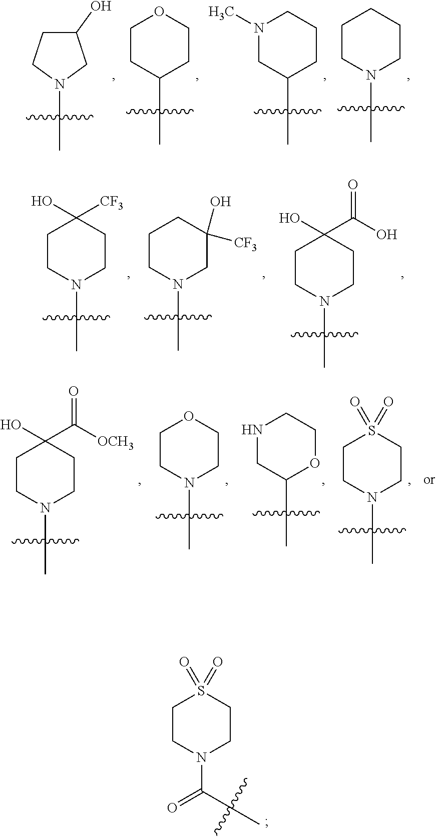

In another aspect, there is provided a compound selected from the exemplified Examples, or a pharmaceutically acceptable salt thereof.

In another aspect, there is provided a compound selected from any subset list of compounds within the scope of any of the above aspects.

The present disclosure also provides a pharmaceutical composition comprising a compound of the present invention, or a pharmaceutically acceptable salt thereof, and a pharmaceutically acceptable carrier.

In another aspect, the present disclosure provides a compound of the present invention, or a pharmaceutically acceptable salt thereof, or a pharmaceutical composition thereof, for use as a medicament.

In another aspect, the present disclosure provides a compound of the present invention, or a pharmaceutically acceptable salt thereof, or a pharmaceutical composition thereof, for use in the manufacture of a medicament for treatment of cancer in a subject in need thereof.

In another aspect, the present disclosure provides a compound of the present invention, or a pharmaceutically acceptable salt thereof, for use in enhancing, stimulating, modulating and/or increasing an immune response in a subject in need thereof, comprising administering to the subject a therapeutically effective amount of said compound or a pharmaceutically acceptable salt thereof.

In another aspect, the present disclosure provides a compound of the present invention, or a pharmaceutically acceptable salt thereof, for use in inhibiting growth, proliferation, or metastasis of cancer cells in a subject in need thereof, comprising administering to the subject a therapeutically effective amount of said compound or a pharmaceutically acceptable salt thereof.

In another aspect, the present disclosure provides a method of enhancing, stimulating, modulating and/or increasing the immune response in a subject in need thereof, said method comprising administering to the subject a therapeutically effective amount of a compound of the present invention, or a pharmaceutically acceptable salt thereof. In a first embodiment of the third aspect, the method further comprises administering an additional agent prior to, after, or simultaneously with the compound of the present invention, or the pharmaceutically acceptable salt thereof. In a second embodiment, the additional agent is an antimicrobial agent, an antiviral agent, a cytotoxic agent, a gene expression modulatory agent, and/or an immune response modifier.

In another aspect, the present disclosure provides a method of inhibiting growth, proliferation, or metastasis of cancer cells in a subject in need thereof, said method comprising administering to the subject a therapeutically effective amount of a compound of the present invention, or a pharmaceutically acceptable salt. In a first embodiment of the fourth aspect, the cancer is selected from melanoma, renal cell carcinoma, squamous non-small cell lung cancer (NSCLC), non-squamous NSCLC, colorectal cancer, castration-resistant prostate cancer, ovarian cancer, gastric cancer, hepatocellular carcinoma, pancreatic carcinoma, squamous cell carcinoma of the head and neck, carcinomas of the esophagus, gastrointestinal tract and breast, and a hematological malignancy.

In another aspect, the present disclosure provides a method of treating an infectious disease in a subject in need thereof, the method comprising administering to the subject a therapeutically effective amount of a compound of the present invention, or a pharmaceutically acceptable salt thereof. In a first embodiment of the fifth aspect, the infectious disease is caused by a virus. In a second embodiment of the fifth aspect, the virus is selected from HIV, Hepatitis A, Hepatitis B, Hepatitis C, Hepatitis D, herpes viruses, papillomaviruses, and influenza.

In another aspect, the present disclosure provides a method of treating septic shock in a subject in need thereof, the method comprising administering to the subject a therapeutically effective amount of a compound of the present invention, or a pharmaceutically acceptable salt thereof.

Unless specifically stated otherwise herein, references made in the singular may also include the plural. For example, “a” and “an” may refer to either one, or one or more. As used herein, the phase “compound(s) or pharmaceutically acceptable salts thereof” refers to at least one compound, at least one salt of the compounds, or a combination thereof. For example, compounds of the present invention or pharmaceutically acceptable salts thereof includes a compound of the present invention; two compounds of the present invention; a salt of a compound of the present invention; a compound of the present invention and one or more salts of the compound of the present invention; and two or more salts of a compound of the present invention.

Unless otherwise indicated, any atom with unsatisfied valences is assumed to have hydrogen atoms sufficient to satisfy the valences.

Throughout the specification, groups and substituents thereof may be chosen by one skilled in the field to provide stable moieties and compounds.

Listed below are definitions of various terms used to describe the present disclosure.

These definitions apply to the terms as they are used throughout the specification (unless they are otherwise limited in specific instances) either individually or as part of a larger group. The definitions set forth herein take precedence over definitions set forth in any patent, patent application, and/or patent application publication incorporated herein by reference.

The term “C₁-C₃ alkyl” as used herein, refers to a group derived from a straight or branched chain saturated hydrocarbon containing from one to three carbon atoms.

The term “C₁-C₆ alkyl” as used herein, refers to a group derived from a straight or branched chain saturated hydrocarbon containing from one to six carbon atoms.

The term “amido” as used herein, refers to —C(O)NH₂.

The term “aminocarbonyl” as used herein, refers to —C(O)NH₂.

The term “carbonyl” as used herein, refers to —C(O)—.

The term “carboxy” as used herein, refers to —CO₂H.

The term “cyano” as used herein, refers to —CN.

The term “cycloalkyl,” as used herein, refers to a group derived from a non-aromatic monocyclic or polycyclic hydrocarbon molecule by removal of one hydrogen atom from a saturated ring carbon atom. Representative examples of cycloalkyl groups include, but are not limited to, cyclopropyl, cyclopentyl, and cyclohexyl. When numbers appear in a subscript after the symbol “C”, the subscript defines with more specificity the number of carbon atoms that a particular cycloalkyl group may contain. For example, “C₃₋₆ cycloalkyl” denotes cycloalkyl groups with three to six carbon atoms.

The term “(C₃-C₆ cycloalkyl)C₁-C₃ alkyl” as used herein, refers to a C₁-C₃ alkyl group substituted with a C₃-C₆ cycloalkyl group.

The terms “halo” and “halogen” as used herein, refer to F, Cl, Br, or I.

The term “C₁-C₄ haloalkoxy” as used herein, refers to a haloC₁-C₄alkyl group attached to the parent molecular moiety through an oxygen atom.

The term “C₁-C₃ haloalkyl” as used herein, refers to a C₁-C₃ alkyl group substituted with one, two, or three halogen atoms.

The term “hydroxyalkyl” includes both branched and straight-chain saturated alkyl groups substituted with one or more hydroxyl groups. For example, “hydroxyalkyl” includes —CH₂OH, —CH₂CH₂OH, and C₁₋₄ hydroxyalkyl.

The term “nitro” as used herein, refers to —NO₂.

The term “oxo” as used herein, refers to ═O.

The term “heteroatom” refers to oxygen (O), sulfur (S), and nitrogen (N).

The terms “heterocyclo”, “heterocyclic”, or “heterocyclyl” may be used interchangeably and refer to cyclic groups having at least one saturated or partially saturated non-aromatic ring and wherein one or more of the rings have at least one heteroatom (O, S or N), said heteroatom containing ring preferably having 1 to 3 heteroatoms independently selected from O, S, and/or N. The ring of such a group containing a heteroatom can contain one or two oxygen or sulfur atoms and/or from one to four nitrogen atoms provided that the total number of heteroatoms in each ring is four or less, and further provided that the ring contains at least one carbon atom. The nitrogen and sulfur atoms may optionally be oxidized and the nitrogen atoms may optionally be quaternized. The heterocyclo group may be attached at any available nitrogen or carbon atom. The heterocyclo ring may be unsubstituted or may contain one or more substituents as valence allows.

Exemplary monocyclic heterocyclyl groups include pyrrolidinyl, imidazolinyl, oxazolidinyl, isoxazolinyl, thiazolidinyl, isothiazolidinyl, tetrahydrofuranyl, piperidinyl, piperazinyl, 2-oxopiperazinyl, 2-oxopiperidinyl, 2-oxopyrrolodinyl, 2-oxoazepinyl, azepinyl, 4-piperidonyl, tetrahydropyranyl, morpholinyl, thiamorpholinyl, thiamorpholinyl sulfoxide, thiamorpholinyl sulfone, 1,3-dioxolane, tetrahydro-1,1-dioxothienyl, dihydroisoindolyl, and tetrahydroquinolinyl.

The term heterocyclyl also encompasses heteroaryl compounds.

Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thiophenyl, oxadiazolyl, pyridinyl, pyrazinyl, pyrimidinyl, pyridazinyl, and triazinyl. Exemplary bicyclic heteroaryl groups include indolyl, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, and pyrrolopyridyl.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds of the present invention can form salts which are also within the scope of this disclosure. Unless otherwise indicated, reference to an inventive compound is understood to include reference to one or more salts thereof. The term “salt(s)” denotes acidic and/or basic salts formed with inorganic and/or organic acids and bases. In addition, the term “salt(s) may include zwitterions (inner salts), e.g., when a compound of the present invention contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps which may be employed during preparation, and thus, are contemplated within the scope of the disclosure. Salts of the compounds of the present invention may be formed, for example, by reacting a compound of the present invention with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, maleates (formed with maleic acid), 2-hydroxyethanesulfonates, lactates, methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts; alkaline earth metal salts such as calcium and magnesium salts; barium, zinc, and aluminum salts; salts with organic bases (for example, organic amines) such as trialkylamines such as triethylamine, procaine, dibenzylamine, N-benzyl-P-phenethylamine, 1-ephenamine, N,N′-dibenzylethylene-diamine, dehydroabietylamine, N-ethylpiperidine, benzylamine, dicyclohexylamine or similar pharmaceutically acceptable amines and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Preferred salts include monohydrochloride, hydrogensulfate, methanesulfonate, phosphate or nitrate salts.

Various forms of prodrugs are well known in the art and are described in:
a) The Practice of Medicinal Chemistry, Camille G. Wermuth et al., Ch 31, (Academic Press, 1996);
b) Design of Prodrugs, edited by H. Bundgaard, (Elsevier, 1985);
c) A Textbook of Drug Design and Development, P. Krogsgaard-Larson and H. Bundgaard, eds. Ch 5, pgs 113-191 (Harwood Academic Publishers, 1991); and
d) Hydrolysis in Drug and Prodrug Metabolism, Bernard Testa and Joachim M. Mayer, (Wiley-VCH, 2003).

Compounds of the present disclosure may contain stereoisomers, wherein asymmetric or chiral centers are present. Specific stereochemistry may be designated by the symbols “R” or “S” depending on the configuration of substituents around the chiral carbon atom. The present invention contemplates various stereoisomers (i.e., enantiomers and diastereomers) and mixtures thereof and is intended to encompass all stereoisomers that bind to PD-L1. Individual stereoisomers of compounds of the present invention may be prepared synthetically from commercially available starting materials which contain asymmetric or chiral centers or by preparation of racemic mixtures followed by resolution well-known to those of ordinary skill in the art.

In addition, compounds of the present invention, subsequent to their preparation, can be isolated and purified to obtain a composition containing an amount by weight equal to or greater than 99% of a compound of the present invention (“substantially pure”), which is then used or formulated as described herein. Such “substantially pure” compounds of the present invention are also contemplated herein as part of the present disclosure.

“Stable compound” and “stable structure” are meant to indicate a compound that is sufficiently robust to survive isolation to a useful degree of purity from a reaction mixture, and formulation into an efficacious therapeutic agent. The present disclosure is intended to embody stable compounds.

“Therapeutically effective amount” is intended to include an amount of a compound of the present disclosure alone or an amount of the combination of compounds claimed or an amount of a compound of the present disclosure in combination with other active ingredients effective to inhibit PD-1/PD-L1 protein/protein and/or CD80/PD-L1 protein/protein interactions, or effective to treat or prevent cancer or infectious disease, such as septic shock, HIV or Hepatitis B, Hepatitis C, and Hepatitis D.

As used herein, “treating” or “treatment” cover the treatment of a disease-state in a mammal, particularly in a human, and may include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

The compounds of the present disclosure are intended to include all isotopes of atoms occurring in the present compounds. Isotopes include those atoms having the same atomic number but different mass numbers. By way of general example and without limitation, isotopes of hydrogen include deuterium (D) and tritium (T). Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. For example, methyl (—CH₃) also includes deuterated methyl groups such as —CD₃.

Compounds in accordance with the present invention and/or pharmaceutically acceptable salts thereof can be administered by any means suitable for the condition to be treated, which can depend on the need for site-specific treatment or quantity of the present invention compound to be delivered. Also embraced within this disclosure is a class of pharmaceutical compositions comprising a compound of the present invention and/or pharmaceutically acceptable salts thereof; and one or more non-toxic, pharmaceutically-acceptable carriers and/or diluents and/or adjuvants (collectively referred to herein as “carrier” materials) and, if desired, other active ingredients. The compounds of the present invention may be administered by any suitable route, preferably in the form of a pharmaceutical composition adapted to such a route, and in a dose effective for the treatment intended. The compounds and compositions of the present disclosure may, for example, be administered orally, mucosally, rectally, or parentally including intravascularly, intravenously, intraperitoneally, subcutaneously, intramuscularly, and intrasternally in dosage unit formulations containing conventional pharmaceutically acceptable carriers, adjuvants, and vehicles. For example, the pharmaceutical carrier may contain a mixture of mannitol or lactose and microcrystalline cellulose. The mixture may contain additional components such as a lubricating agent, e.g. magnesium stearate and a disintegrating agent such as crospovidone. The carrier mixture may be filled into a gelatin capsule or compressed as a tablet. The pharmaceutical composition may be administered as an oral dosage form or an infusion, for example.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg, preferably from about 0.25 to 250 mg, and more preferably from about 0.5 to 100 mg. A suitable daily dose for a human or other mammal may vary widely depending on the condition of the patient and other factors, but, can be determined using routine methods.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the disclosure can contain at least one agent selected from sweetening agents, flavoring agents, coloring agents, demulcents, antioxidants, and preserving agents.

A tablet can, for example, be prepared by admixing at least one compound of the present invention and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials, include, but are not limited to, hydroxypropyl-methylcellulose and hydroxypropyl-cellulose. Exemplary time delay materials, include, but are not limited to, ethyl cellulose and cellulose acetate butyrate.

Hard gelatin capsules can, for example, be prepared by mixing at least one compound of the present invention and/or at least one salt thereof with at least one inert solid diluent, such as, for example, calcium carbonate; calcium phosphate; and kaolin.

Soft gelatin capsules can, for example, be prepared by mixing at least one compound of the present invention and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of the present invention and/or at least one pharmaceutically acceptable salt thereof with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of the present invention and/or at least one pharmaceutically acceptable salt thereof in either a vegetable oil, such as, for example, arachis oil; olive oil; sesame oil; and coconut oil; or in mineral oil, such as, for example, liquid paraffin. An oily suspension can also contain at least one thickening agent, such as, for example, beeswax; hard paraffin; and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of the present invention and/or at least one pharmaceutically acceptable salt thereof with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents; flavoring agents; and coloring agents.

An emulsion of at least one compound of the present invention and/or at least one pharmaceutically acceptable salt thereof can, for example, be prepared as an oil-in-water emulsion. The oily phase of the emulsions comprising compounds of the present invention may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soy bean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present disclosure include Tween 60, Span 80, cetostearyl alcohol, myristyl alcohol, glyceryl monostearate, sodium lauryl sulfate, glyceryl distearate alone or with a wax, or other materials well known in the art.

The compounds of the present invention and/or at least one pharmaceutically acceptable salt thereof can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions; and aqueous or oleaginous suspensions.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound of the present invention in an oily phase, such as, for example, a mixture of soybean oil and lecithin; 2) combining the present invention containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers, adjuvants, and vehicles that may be used in the pharmaceutical compositions of this disclosure include, but are not limited to, ion exchangers, alumina, aluminum stearate, lecithin, self-emulsifying drug delivery systems (SEDDS) such as d-alpha-tocopherol polyethyleneglycol 1000 succinate, surfactants used in pharmaceutical dosage forms such as Tweens, polyethoxylated castor oil such as CREMOPHOR surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this disclosure can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

The amounts of compounds that are administered and the dosage regimen for treating a disease condition with the compounds and/or compositions of this disclosure depends on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.0025 and about 50 mg/kg body weight and most preferably between about 0.005 to 10 mg/kg body weight, may be appropriate. The daily dose can be administered in one to four doses per day. Other dosing schedules include one dose per week and one dose per two day cycle.

For therapeutic purposes, the active compounds of this disclosure are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Pharmaceutical compositions of this disclosure comprise at least one compound of the present invention and/or at least one pharmaceutically acceptable salt thereof, and optionally an additional agent selected from any pharmaceutically acceptable carrier, adjuvant, and vehicle. Alternate compositions of this disclosure comprise a compound of the present invention described herein, or a prodrug thereof, and a pharmaceutically acceptable carrier, adjuvant, or vehicle.

The compounds of the disclosure inhibit the PD-1/PD-L1 protein/protein resulting in a PD-L1 blockade. The blockade of PD-L1 can enhance the immune response to cancerous cells and infectious diseases in mammals, including humans.

In one aspect, the present disclosure relates to treatment of a subject in vivo using a compound of the present invention or a salt thereof such that growth of cancerous tumors is inhibited. A compound of the present invention or a salt thereof may be used alone to inhibit the growth of cancerous tumors. Alternatively, a compound of the present invention or a salt thereof may be used in conjunction with other immunogenic agents or standard cancer treatments, as described below.

In one embodiment, the disclosure provides a method of inhibiting growth of tumor cells in a subject, comprising administering to the subject a therapeutically effective amount of a compound of the present invention or a salt thereof.

In one embodiment, a method is provided for treating cancer comprising administering to a patient in need thereof, a therapeutically effective amount of a compound of the present invention or a salt thereof. Examples of cancers include those whose growth may be inhibited using compounds of the disclosure include cancers typically responsive to immunotherapy. Non-limiting examples of preferred cancers for treatment include melanoma (e.g., metastatic malignant melanoma), renal cancer (e.g. clear cell carcinoma), prostate cancer (e.g. hormone refractory prostate adenocarcinoma), breast cancer, colon cancer and lung cancer (e.g. non-small cell lung cancer). Additionally, the disclosure includes refractory or recurrent malignancies whose growth may be inhibited using the compounds of the disclosure.

Examples of other cancers that may be treated using the methods of the disclosure include bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The present disclosure is also useful for treatment of metastatic cancers, especially metastatic cancers that express PD-L1 (Iwai et al. (2005) Int. Immunol. 17:133-144).

Optionally, the compounds of the present invention or salts thereof can be combined with another immunogenic agent, such as cancerous cells, purified tumor antigens (including recombinant proteins, peptides, and carbohydrate molecules), cells, and cells transfected with genes encoding immune stimulating cytokines (He et al (2004) J. Immunol. 173:4919-28). Non-limiting examples of tumor vaccines that can be used include peptides of melanoma antigens, such as peptides of gp100, MAGE antigens, Trp-2, MART1 and/or tyrosinase, or tumor cells transfected to express the cytokine GM-CSF.

In humans, some tumors have been shown to be immunogenic such as melanomas. It is anticipated that by raising the threshold of T cell activation by PD-L1 blockade, tumor responses are expected to be activated in the host.

The PD-L1 blockade can be combined with a vaccination protocol. Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita, V. et al. (eds.), 1997, Cancer: Principles and Practice of Oncology. Fifth Edition). In one of these strategies, a vaccine is prepared using autologous or allogenenic tumor cells. These cellular vaccines have been shown to be most effective when the tumor cells are transduced to express GM-CSF. GM-CSF has been shown to be a potent activator of antigen presentation for tumor vaccination (Dranoff et al. (1993) Proc. Natl. Acad. Sci. U.S.A. 90: 3539-43).

The study of gene expression and large scale gene expression patterns in various tumors has led to the definition of so called tumor specific antigens (Rosenberg, S A (1999) Immunity 10: 281-7). In many cases, these tumor specific antigens are differentiation antigens expressed in the tumors and in the cell from which the tumor arose, for example melanocyte antigens gp100, MAGE antigens, and Trp-2. More importantly, many of these antigens can be shown to be the targets of tumor specific T cells found in the host. PD-L1 blockade may be used in conjunction with a collection of recombinant proteins and/or peptides expressed in a tumor in order to generate an immune response to these proteins. These proteins are normally viewed by the immune system as self antigens and are therefore tolerant to them. The tumor antigen may also include the protein telomerase, which is required for the synthesis of telomeres of chromosomes and which is expressed in more than 85% of human cancers and in only a limited number of somatic tissues (Kim, N et al. (1994) Science 266: 2011-2013). (These somatic tissues may be protected from immune attack by various means). Tumor antigen may also be “neo-antigens” expressed in cancer cells because of somatic mutations that alter protein sequence or create fusion proteins between two unrelated sequences (ie. bcr-abl in the Philadelphia chromosome), or idiotype from B cell tumors.

Other tumor vaccines may include the proteins from viruses implicated in human cancers such a Human Papilloma Viruses (HPV), Hepatitis Viruses (HBV, HDV and HCV) and Kaposi's Herpes Sarcoma Virus (KHSV). Another form of tumor specific antigen which may be used in conjunction with PD-L1 blockade is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot, R & Srivastava, P (1995) Science 269:1585-1588; Tamura, Y. et al. (1997) Science 278:117-120).

Dendritic cells (DC) are potent antigen presenting cells that can be used to prime antigen-specific responses. DC's can be produced ex vivo and loaded with various protein and peptide antigens as well as tumor cell extracts (Nestle, F. et al. (1998) Nature Medicine 4: 328-332). DCs may also be transduced by genetic means to express these tumor antigens as well. DCs have also been fused directly to tumor cells for the purposes of immunization (Kugler, A. et al. (2000) Nature Medicine 6:332-336). As a method of vaccination, DC immunization may be effectively combined with PD-L1 blockade to activate more potent anti-tumor responses.

PD-L1 blockade may also be combined with standard cancer treatments. PD-L1 blockade may be effectively combined with chemotherapeutic regimes. In these instances, it may be possible to reduce the dose of chemotherapeutic reagent administered (Mokyr, M. et al. (1998) Cancer Research 58: 5301-5304). An example of such a combination is a compound of this disclosure in combination with dacarbazine for the treatment of melanoma. Another example of such a combination is a compound of this disclosure in combination with interleukin-2 (IL-2) for the treatment of melanoma. The scientific rationale behind the combined use of PD-L1 blockade and chemotherapy is that cell death, that is a consequence of the cytotoxic action of most chemotherapeutic compounds, should result in increased levels of tumor antigen in the antigen presentation pathway. Other combination therapies that may result in synergy with PD-L1 blockade through cell death are radiation, surgery, and hormone deprivation. Each of these protocols creates a source of tumor antigen in the host. Angiogenesis inhibitors may also be combined with PD-L1 blockade. Inhibition of angiogenesis leads to tumor cell death which may feed tumor antigen into host antigen presentation pathways.

The compounds of this disclosure can also be used in combination with bispecific compounds that target Fc alpha or Fc gamma receptor-expressing effectors cells to tumor cells (see, e.g., U.S. Pat. Nos. 5,922,845 and 5,837,243). Bispecific compounds can be used to target two separate antigens. For example anti-Fc receptor/anti tumor antigen (e.g., Her-2/neu) bispecific compounds have been used to target macrophages to sites of tumor. This targeting may more effectively activate tumor specific responses. The T cell arm of these responses would be augmented by the use of PD-L1 blockade. Alternatively, antigen may be delivered directly to DCs by the use of bispecific compounds which bind to tumor antigen and a dendritic cell specific cell surface marker.

Tumors evade host immune surveillance by a large variety of mechanisms. Many of these mechanisms may be overcome by the inactivation of proteins which are expressed by the tumors and which are immunosuppressive. These include among others TGF-beta (Kehrl, J. et al. (1986) J Exp. Med. 163: 1037-1050), IL-10 (Howard, M. & O'Garra, A. (1992) Immunology Today 13: 198-200), and Fas ligand (Hahne, M. et al. (1996) Science 274: 1363-1365). Inhibitors that bind to and block each of these entities may be used in combination with the compounds of this disclosure to counteract the effects of the immunosuppressive agent and favor tumor immune responses by the host.

Compounds that activate host immune responsiveness can be used in combination with PD-L1 blockade. These include molecules on the surface of dendritic cells which activate DC function and antigen presentation. Anti-CD40 compounds are able to substitute effectively for T cell helper activity (Ridge, J. et al. (1998) Nature 393: 474-478) and can be used in conjunction with PD-L1 blockade (Ito, N. et al. (2000) Immunobiology 201 (5) 527-40). Activating compounds to T cell costimulatory molecules such as CTLA-4 (e.g., U.S. Pat. No. 5,811,097), OX-40 (Weinberg, A. et al. (2000) Immunol 164: 2160-2169), 4-1BB (Melero, I. et al. (1997) Nature Medicine 3: 682-685 (1997), and ICOS (Hutloff, A. et al. (1999) Nature 397: 262-266) may also provide for increased levels of T cell activation.

Bone marrow transplantation is currently being used to treat a variety of tumors of hematopoietic origin. While graft versus host disease is a consequence of this treatment, therapeutic benefit may be obtained from graft vs. tumor responses. PD-L1 blockade can be used to increase the effectiveness of the donor engrafted tumor specific T cells.

Other methods of the disclosure are used to treat patients who have been exposed to particular toxins or pathogens. Accordingly, another aspect of the disclosure provides a method of treating an infectious disease in a subject comprising administering to the subject a therapeutically effective amount of a compound of the present invention or salts thereof.

Similar to its application to tumors as discussed above, the compound of the present invention or salts thereof can be used alone, or as an adjuvant, in combination with vaccines, to stimulate the immune response to pathogens, toxins, and self-antigens. Examples of pathogens for which this therapeutic approach may be particularly useful, include pathogens for which there is currently no effective vaccine, or pathogens for which conventional vaccines are less than completely effective. These include, but are not limited to HIV, Hepatitis (A, B, C or D), Influenza, Herpes, Giardia, Malaria, Leishmania, Staphylococcus aureus, Pseudomonas Aeruginosa. PD-L1 blockade is particularly useful against established infections by agents such as HIV that present altered antigens over the course of the infections. These novel epitopes are recognized as foreign at the time of administration, thus provoking a strong T cell response that is not dampened by negative signals through PD-1.

Some examples of pathogenic viruses causing infections treatable by methods of the disclosure include HIV, hepatitis (A, B, C, or D), herpes viruses (e.g., VZV, HSV-1, HAV-6, HHv-7, HHV-8, HSV-2, CMV, and Epstein Barr virus), adenovirus, influenza virus, flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Some examples of pathogenic bacteria causing infections treatable by methods of the disclosure include chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lymes disease bacteria.

Some examples of pathogenic fungi causing infections treatable by methods of the disclosure include Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Some examples of pathogenic parasites causing infections treatable by methods of the disclosure include Entamoeba histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

In all of the above methods, PD-L1 blockade can be combined with other forms of immunotherapy such as cytokine treatment (e.g., interferons, GM-CSF, G-CSF, IL-2), or bispecific antibody therapy, which provides for enhanced presentation of tumor antigens (see, e.g., Holliger (1993) Proc. Natl. Acad. Sci. USA 90:6444-6448; Poljak (1994) Structure 2:1121-1123), vaccines, or agents that modify gene expression.

The compounds of this disclosure may provoke and amplify autoimmune responses. Indeed, induction of anti-tumor responses using tumor cell and peptide vaccines reveals that many anti-tumor responses involve anti-self reactivities (depigmentation observed in anti-CTLA-4+GM-CSF-modified B 16 melanoma in van Elsas et al. supra; depigmentation in Trp-2 vaccinated mice (Overwijk, W. et al. (1999) Proc. Nat. Acad. Sci. U.S.A. 96: 2982-2987); autoimmune prostatitis evoked by TRAMP tumor cell vaccines (Hurwitz, A. (2000) supra), melanoma peptide antigen vaccination and vitilago observed in human clinical trials (Rosenberg, S A and White, D E (1996) J. Immunother Emphasis Tumor Immunol 19 (1): 81-4).

Therefore, it is possible to consider using anti-PD-L1 blockade in conjunction with various self proteins in order to devise vaccination protocols to efficiently generate immune responses against these self proteins for disease treatment. For example, Alzheimer's disease involves inappropriate accumulation of A.beta.peptide in amyloid deposits in the brain; antibody responses against amyloid are able to clear these amyloid deposits (Schenk et al., (1999) Nature 400: 173-177).

Other self proteins may also be used as targets such as IgE for the treatment of allergy and asthma, and TNF.alpha. for rheumatoid arthritis. Finally, antibody responses to various hormones may be induced by the use of a compound of the present invention or salts thereof. Neutralizing antibody responses to reproductive hormones may be used for contraception. Neutralizing antibody response to hormones and other soluble factors that are required for the growth of particular tumors may also be considered as possible vaccination targets.

Analogous methods as described above for the use of anti-PD-L1 antibody can be used for induction of therapeutic autoimmune responses to treat patients having an inappropriate accumulation of other self-antigens, such as amyloid deposits, including A.beta. in Alzheimer's disease, cytokines such as TNF alpha, and IgE.

The compounds of this disclosure may be used to stimulate antigen-specific immune responses by co-administration of a compound of the present invention or salts thereof with an antigen of interest (e.g., a vaccine). Accordingly, in another aspect the disclosure provides a method of enhancing an immune response to an antigen in a subject, comprising administering to the subject: (i) the antigen; and (ii) a compound of the present invention or salts thereof, such that an immune response to the antigen in the subject is enhanced. The antigen can be, for example, a tumor antigen, a viral antigen, a bacterial antigen or an antigen from a pathogen. Non-limiting examples of such antigens include those discussed in the sections above, such as the tumor antigens (or tumor vaccines) discussed above, or antigens from the viruses, bacteria or other pathogens described above.

As previously described, the compounds of the disclosure can be co-administered with one or more other therapeutic agents, e.g., a cytotoxic agent, a radiotoxic agent or an immunosuppressive agent. The compounds of the disclosure can be administered before, after or concurrently with the other therapeutic agent or can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil, decarbazine and cyclophosphamide hydroxyurea which, by themselves, are only effective at levels which are toxic or subtoxic to a patient. Cisplatin is intravenously administered as a 100 mg/dose once every four weeks and adriamycin is intravenously administered as a 60-75 mg/mL dose once every 21 days. Co-administration of a compound of the present invention or salts thereof, with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody.

Also within the scope of the present disclosure are kits comprising a compound of the present invention or salts thereof and instructions for use. The kit can further contain at least one additional reagent. Kits typically include a label indicating the intended use of the contents of the kit. The term label includes any writing, or recorded material supplied on or with the kit, or which otherwise accompanies the kit.

The above other therapeutic agents, when employed in combination with the compounds of the present disclosure, may be used, for example, in those amounts indicated in the Physicians' Desk Reference (PDR) or as otherwise determined by one of ordinary skill in the art. In the methods of the present disclosure, such other therapeutic agent(s) may be administered prior to, simultaneously with, or following the administration of the inventive compounds.

### EXAMPLES

The invention is further defined in the following Examples. It should be understood that the Examples are given by way of illustration only. From the above discussion and the Examples, one skilled in the art can ascertain the essential characteristics of the invention, and without departing from the spirit and scope thereof, can make various changes and modifications to adapt the invention to various uses and conditions. As a result, the invention is not limited by the illustrative examples set forth hereinbelow, but rather is defined by the claims appended hereto.

The compounds may be made by methods known in the art including those described below and including variations within the skill of the art. Some reagents and intermediates are known in the art. Other reagents and intermediates can be made by methods known in the art using readily available materials. The variables (e.g. numbered “R” substituents) used to describe the synthesis of the compounds are intended only to illustrate how to make the compounds and are not to be confused with variables used in the claims or in other sections of the specification. The following methods are for illustrative purposes and are not intended to limit the scope of the invention.

Abbreviations used in the schemes generally follow conventions used in the art. Chemical abbreviations used in the specification and examples are defined as follows: “THF” for tetrahydrofuran; “DMF” for N,N-dimethylformamide; “MeOH” for methanol; “EtOH” for ethanol; “n-PrOH” for 1-propyl alcohol or propan-1-ol; “i-PrOH” for 2-propyl alcohol or propan-2-ol; “Ar” for aryl; “TFA” for trifluoroacetic acid; “DMSO” for dimethylsulfoxide; “EtOAc” for ethyl acetate; “Et₂O” for diethyl ether; “DMAP” for 4-dimethylaminopyridine; “DCE” for 1,2-dichloroethane; “ACN” for acetonitrile; “DME” for 1,2-dimethoxyethane; “h” for hours; “rt” for room temperature or retention time (context will dictate); “min” for minutes; “HOBt” for 1-hydroxybenzotriazole hydrate; “HCTU” for 1-[bis(dimethylamino)methylen]-5-chlorobenzotriazolium 3-oxide hexafluorophosphate or N,N,N′,N-tetramethyl-O-(6-chloro-1H-benzotriazol-1-yl)uronium hexafluorophosphate; “HATU” for 1-[bis(dimethylamino)methylene]-1H-1,2,3-triazolo[4,5-b]pyridinium 3-oxid hexafluorophosphate or N-[(dimethylamino)-1H-1,2,3-triazolo-[4,5-b]pyridin-1-ylmethylene]-N-methylmethanaminium hexafluorophosphate N-oxide; “DIEA” and “iPrNEt₂” for diisopropylethylamine; “Et₃N” for triethyl amine.

Abbreviations are defined as follows: “1×” for once, “2×” for twice, “3×” for thrice, “C” for degrees Celsius, “eq” for equivalent or equivalents, “g” for gram or grams, “mg” for milligram or milligrams, “L” for liter or liters, “mL” for milliliter or milliliters, “μL” for microliter or microliters, “N” for normal, “M” for molar, “mmol” for millimole or millimoles, “min” for minute or minutes, “h” for hour or hours, “rt” for room temperature, “RT” for retention time, “atm” for atmosphere, “psi” for pounds per square inch, “conc.” for concentrate, “sat” or “sat'd” for saturated, “MW” for molecular weight, “mp” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry, “LC” for liquid chromatography, “LCMS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy, “¹H” for proton, “6” for delta, “s” for singlet, “d” for doublet, “t” for triplet, “q” for quartet, “m” for multiplet, “br” for broad, “Hz” for hertz, and “α”, “β”, “R”, “S”, “E”, and “Z” are stereochemical designations familiar to one skilled in the art.

### Analytical LCMS Conditions for Example 1001 to Example 1020

Condition ACN-AA: Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm.

Condition ACN-TFA: Column: Waters Aquity UPLC BEHC18, 2.1×50 mm, 1.7-μm particles; Mobile Phase A: 99.95% water with 0.05% trifluoroacetic acid; Mobile Phase B: 99.95% acetonitrile with 0.05% trifluoroacetic acid; Gradient: 2% B, 2-98% B over 1 minutes, then a 0.5-minute hold at 98% B; Flow: 0.8 mL/min; Detection: UV at 220 nm or 254 nm.

Condition MeOH-AA: Column: Waters BEH C18, 2.0×50 mm, 1.7-μm particles; Mobile Phase A: 5:95 methanol:water with 10 mM ammonium acetate; Mobile Phase B: 95:5 methanol:water with 10 mM ammonium acetate; Temperature: 50° C.; Gradient: 0% B, 0-100% B over 3 minutes, then a 0.5-minute hold at 100% B; Flow: 0.5 mL/min; Detection: UV at 220 nm. Proton NMR was acquired in deuterated DMSO.

### Intermediate: 2-(Chloromethyl)-5-methylpyrazine hydrochloride

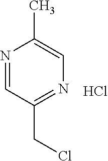

A solution of (5-methylpyrazin-2-yl)methanol (0.094 g, 0.757 mmol) in dichloromethane (5 mL) was treated with thionyl chloride (0.332 mL, 4.54 mmol) and stirred for 3 hours. The reaction was concentrated, and then dried under vacuum pump for 30 minutes to afford the product as a pale orange solid, which was used immediately in the following reaction.

### Intermediate: 5-Chloro-4-((3-(2,3-dihydrobenzo[b][1,4]dioxin-6-yl)-2-methylbenzyl)oxy)-2-((5-methylpyrazin-2-yl)methoxy)benzaldehyde

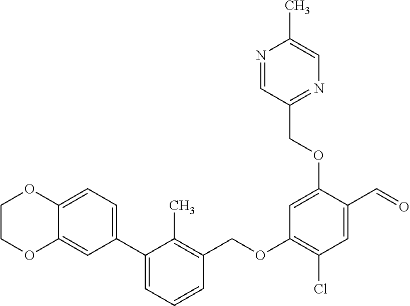 [SMILES: Cc1cnc(COc2cc(OCc3cccc(-c4ccc5c(c4)OCCO5)c3C)c(Cl)cc2C=O)cn1]

A stirred mixture of 5-chloro-4-((3-(2,3-dihydrobenzo[b][1,4]dioxin-6-yl)-2-methylbenzyl)oxy)-2-hydroxybenzaldehyde (0.224 g, 0.545 mmol), 2-(chloromethyl)-5-methylpyrazine hydrochloride (0.125 g, 0.70 mmol), cesium carbonate (0.463 g, 1.421 5 mmol) and sodium iodide (8 mg, 0.054 mmol) in N,N-dimethylformamide (3 mL) was heated at 75° C. for 1.5 hours. The reaction was cooled, then water was added and a large solid mass formed. The solid did not dissolve significantly in ethyl acetate, so a large volume of dichloromethane was used to dissolve the solids. The separate organic layers were washed with water and then combined and dried over sodium sulfate, filtered, and concentrated under reduced pressure. The orange residue was dissolved once in minimal dichloromethane and dried by rotary evaporator. The solids were triturated with diethyl ether, collecting the product (0.250 g, 0.484 mmol, 90% yield) by vacuum filtration, after air drying. The material was used without purification in following experiments. LCMS (Condition ACN-TFA, ES+) M+H=517.1, 1.16 minutes, calculated exact mass=516.15. ¹H NMR (400 MHz, CDCl₃) δ: 10.36 (s, 1H), 8.70 (s, 1H), 8.45 (s, 1H), 7.91 (s, 1H), 7.43 (t, J=4.5 Hz, 1H), 7.26 (d, J=4.3 Hz, 2H), 6.93 (d, J=8.3 Hz, 1H), 6.85 (d, J=2.0 Hz, 1H), 6.82-6.77 (m, 2H), 5.32 (s, 2H), 5.21 (s, 2H), 4.32 (s, 4H), 2.61 (s, 3H), 2.30 (s, 3H).

### Example 1001: (R)-2-((5-Chloro-4-((3-(2,3-dihydrobenzo[b][1,4]dioxin-6-yl)-2-methylbenzyl)oxy)-2-((5-methylpyrazin-2-yl)methoxy)benzyl)amino)-3-hydroxypropanoic acid

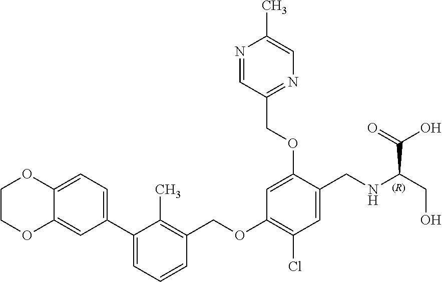

A solution of 5-chloro-4-((3-(2,3-dihydrobenzo[b][1,4]dioxin-6-yl)-2-methylbenzyl)oxy)-2-((5-methylpyrazin-2-yl)methoxy)benzaldehyde (0.039 g, 0.075 mmol) and (R)-2-amino-3-hydroxypropanoic acid (0.024 g, 0.226 mmol) in N,N-dimethylformamide (1 mL) was treated with sodium triacetoxyborohydride (0.050 g, 0.234 mmol). The mixture was stirred for 72 hours. The reaction was diluted with ethyl acetate (5 mL) and washed with saturated aqueous sodium bicarbonate solution (5 mL). The organic layer was dried over sodium sulfate, filtered, and concentrated under reduced pressure. The crude material was purified via preparative LCMS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-100% B over 20 minutes, then a 5-minute hold at 100% B; Flow: 20 mE/minutes. Fractions containing the desired product were combined and dried via centrifugal evaporation, affording the product (0.0096 g, 0.016 mmol. 20.6% yield), and its estimated purity by LCMS analysis was 98%. LCMS (Condition ACN-AA, ES+) M+H=606.5, 1.63 minutes, calculated exact mass=605.19. ¹H NMR (500 MHz, DMSO-d₆) δ: 8.77 (s, 1H), 8.53 (s, 1H), 7.50 (s, 1H), 7.43 (d, J=7.7 Hz, 1H), 7.28-7.20 (m, 1H), 7.19-7.12 (m, 2H), 6.91 (d, J=8.1 Hz, 1H), 6.79-6.70 (m, 2H), 5.36 (s, 2H), 5.25 (s, 2H), 4.27 (s, 4H), 4.00 (s, 2H), 3.76-3.58 (m, 2H), 3.19 (t, J=5.5 Hz, 1H), 2.22 (s, 3H): Note: one methyl signal was solvent obscured.

Examples 1002 and 1003 were prepared in substantially the same manner as Example 1001, using the appropriate amine for the reductive amination.

### Example 1002: (S)-2-((5-Chloro-4-((3-(2,3-dihydrobenzo[b][1,4]dioxin-6-yl)-2-methylbenzyl)oxy)-2-((5-methylpyrazin-2-yl)methoxy)benzyl)amino)-3-hydroxy-2-methylpropanoic acid

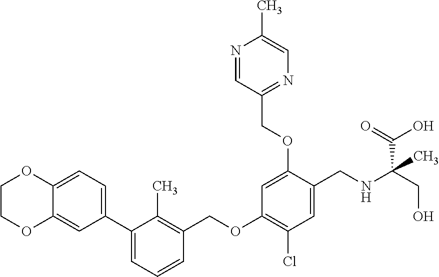 [SMILES: Cc1cnc(COc2cc(OCc3cccc(-c4ccc5c(c4)OCCO5)c3C)c(Cl)cc2CN[C@@](C)(CO)C(=O)O)cn1]

The yield of the product was 1.0 mg, and its estimated purity by LCMS analysis was 99% (LCMS Condition MeOH-AA, ES+) M+H=620.5, 2.80 minutes, calculated exact mass=619.21. ¹H NMR (500 MHz, DMSO-d₆) δ: 8.75 (s, 1H), 8.53 (s, 1H), 7.48-7.38 (m, 2H), 7.26-7.19 (m, 1H), 7.16 (d, J=7.7 Hz, 1H), 7.11 (s, 1H), 6.92 (d, J=8.1 Hz, 1H), 6.80-6.71 (m, 2H), 5.33 (s, 2H), 5.23 (s, 2H), 4.28 (s, 4H), 3.80-3.65 (m, 2H), 2.23 (s, 3H), 1.83 (s, 2H), 1.11 (s, 3H); a methyl signal is partially solvent obscured.

### Intermediate: 4-(Chloromethyl)-1-ethyl-1H-pyrazole hydrochloride

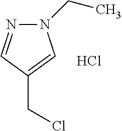

A solution of (1-ethyl-1H-pyrazol-4-yl)methanol (0.050 g, 0.4 mmol) in dichloromethane (5 mL) was treated with thionyl chloride (0.17 mL, 2.4 mmol) and stirred for 3 hours. The reaction was concentrated, and then dried under vacuum pump for 30 minutes to afford the product as clear viscous oil which was used immediately in the following reaction. ¹H NMR (500 MHz, CDCl₃) δ: 7.87 (s, 1H), 7.73 (s, 1H), 4.58 (q, J=7.3 Hz, 2H), 4.55 (s, 2H), 1.66 (t, J=7.3 Hz, 3H).

### Intermediate: 5-Chloro-4-((3-(2,3-dihydrobenzo[b][1,4]dioxin-6-yl)-2-methylbenzyl)oxy)-2-((1-ethyl-1H-pyrazol-4-yl)methoxy)benzaldehyde

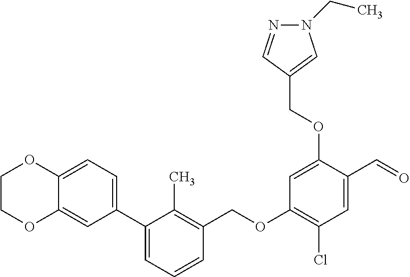 [SMILES: CCn1cc(COc2cc(OCc3cccc(-c4ccc5c(c4)OCCO5)c3C)c(Cl)cc2C=O)cn1]

A stirred mixture of 5-chloro-4-((3-(2,3-dihydrobenzo[b][1,4]dioxin-6-yl)-2-methylbenzyl)oxy)-2-hydroxybenzaldehyde (0.125 g, 0.305 mmol), 4-(chloromethyl)-1-ethyl-1H-pyrazole hydrochloride (0.072 g, 0.396 mmol), cesium carbonate (0.298 g, 0.914 mmol) and sodium iodide (5 mg, 0.030 mmol) in N,N-dimethylformamide (3 mL) was heated at 75° C. for 1.5 hours. The mixture was then cooled, diluted with dichloromethane, and washed with water. The organic layer was dried over sodium sulfate, filtered, and concentrated under reduced pressure, affording an orange oil which solidified upon standing. The solid was triturated with diethyl ether, and the solids were collected by decanting the ether, and then dried under vacuum to afford the product (0.093 g, 0.179 mmol, 58.8% yield) as an orange powder. This material was used directly in the following experiment. LCMS (Condition ACN-TFA, ES+) M+H=519.2, 1.15 minutes, calculated exact mass=518.16. ¹H NMR (500 MHz, CDCl₃) δ: 10.25 (s, 1H), 7.88 (s, 1H), 7.54 (s, 1H), 7.45 (s, 2H), 6.92 (d, J=8.2 Hz, 1H), 6.83 (d, J=1.5 Hz, 1H), 6.78 (d, J=8.4 Hz, 1H), 6.69 (s, 1H), 5.24 (s, 2H), 5.08 (s, 2H), 4.32 (s, 4H), 4.18 (q, J=7.3 Hz, 2H), 2.31 (s, 3H), 1.50 (t, J=7.2 Hz, 3H).

### Example 1003: (S)-2-((5-Chloro-4-((3-(2,3-dihydrobenzo[b][1,4]dioxin-6-yl)-2-methylbenzyl)oxy)-2-((1-ethyl-1H-pyrazol-4-yl)methoxy)benzyl)amino)-3-hydroxy-2-methylpropanoic acid

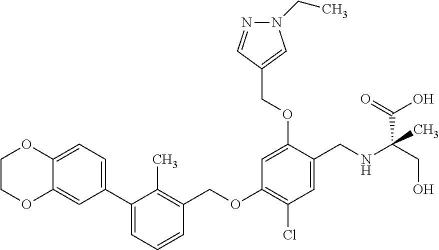 [SMILES: CCn1cc(COc2cc(OCc3cccc(-c4ccc5c(c4)OCCO5)c3C)c(Cl)cc2CN[C@@](C)(CO)C(=O)O)cn1]

The yield of the product was 23.4 mg, and its estimated purity by LCMS analysis was 99% (LCMS Condition MeOH-AA, ES+) M+H=622.5, 2.81 minutes, calculated exact mass=621.22. ¹H NMR (500 MHz, DMSO-d₆) δ: 7.92 (s, 1H), 7.55 (s, 1H), 7.51-7.43 (m, 2H), 7.33-7.22 (m, 1H), 7.18 (d, J=7.7 Hz, 1H), 7.14 (s, 1H), 6.92 (d, J=8.4 Hz, 1H), 6.82-6.69 (m, 2H), 5.29 (s, 2H), 5.11 (s, 2H), 4.28 (s, 4H), 4.09 (q, J=7.3 Hz, 2H), 3.88 (s, 2H), 3.61 (d, J=11.4 Hz, 1H), 3.51 (d, J=11.4 Hz, 1H), 2.89 (s, 1H), 2.73 (s, 1H), 2.24 (s, 3H), 1.90 (s, 1H), 1.34 (t, J=7.3 Hz, 3H), 1.22 (s, 3H).

### Example 1004: (S)-2-((5-Chloro-4-((3-(2,3-dihydrobenzo[b][1,4]dioxin-6-yl)-2-methylbenzyl)oxy)-2-((2-(morpholine-4-carboxamido)pyridin-4-yl)methoxy)benzyl)amino)-3-hydroxy-2-methylpropanoic acid

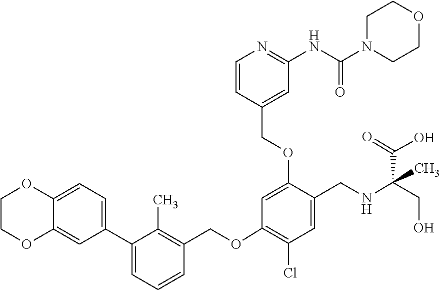 [SMILES: Cc1c(COc2cc(OCc3ccnc(NC(=O)N4CCOCC4)c3)c(CN[C@@](C)(CO)C(=O)O)cc2Cl)cccc1-c1ccc2c(c1)OCCO2]

This example was prepared in a similar manner as Example 1001, using the appropriate amine for the reductive amination. The yield of the product was 1.4 mg, and its estimated purity by LCMS analysis was 98% (LCMS Condition ACN-AA, ES+) M+H=733.3, 1.65 minutes, calculated exact mass=732.26. ¹H NMR (400 MHz, CDCl₃) δ: 8.10 (d, J=4.4 Hz, 1H), 7.97 (br. s., 1H), 7.45 (br. s., 1H), 7.31 (br. s., 1H), 7.16 (s, 2H), 7.01 (d, J=3.9 Hz, 1H), 6.87 (d, J=8.3 Hz, 1H), 6.77 (s, 1H), 6.71 (d, J=8.1 Hz, 1H), 6.48 (br. s., 1H), 5.02 (br. s., 2H), 4.90 (br. s., 2H), 4.28 (s, 5H), 3.94 (br. s., 2H), 3.71 (br. s., 2H), 3.63 (br. s., 5H), 3.44 (br. s., 4H), 2.15 (s, 3H), 1.27 (br. s., 3H).

### Example 1005: (R)-2-((5-Chloro-4-((3-(2,3-dihydrobenzo[b][1,4]dioxin-6-yl)-2-methylbenzyl)oxy)-2-((1-ethyl-1H-pyrazol-4-yl)methoxy)benzyl)amino)-3-hydroxypropanoic acid

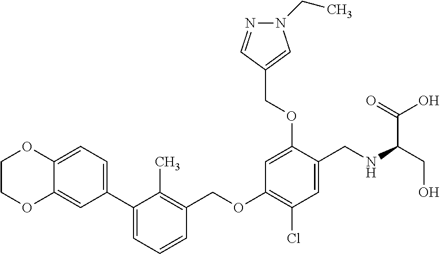 [SMILES: CCn1cc(COc2cc(OCc3cccc(-c4ccc5c(c4)OCCO5)c3C)c(Cl)cc2CN[C@H](CO)C(=O)O)cn1]

A suspension of 5-chloro-4-((3-(2,3-dihydrobenzo[b][1,4]dioxin-6-yl)-2-methylbenzyl)oxy)-2-((1-ethyl-1H-pyrazol-4-yl)methoxy)benzaldehyde (0.039 g, 0.075 mmol) and (R)-2-amino-3-hydroxypropanoic acid (0.012 g, 0.113 mmol) in dry N,N-dimethylformamide (0.9 mL) and glacial acetic acid (0.100 mL) was stirred for 20 minutes and then treated with borane-2-picoline complex (0.016 g, 0.150 mmol). The mixture was stirred for 16 hours then diluted with water and ethyl acetate, and quenched by addition of saturated aqueous sodium bicarbonate solution. The organic layer was concentrated and the residue was purified via preparative LCMS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 35-75% B over 15 minutes, then a 5-minute hold at 100% B; Flow: 20 mL/min. Fractions containing the desired product were combined and dried via centrifugal evaporation. The yield of the product was 19.9 mg, and its estimated purity by LCMS analysis was 98% (LCMS Condition MeOH-AA, ESI+) M+H=608.15, 3.28 minutes, calculated exact mass=607.21. ¹H NMR (500 MHz, DMSO-d₆) δ: 7.92 (s, 1H), 7.58 (s, 1H), 7.51-7.42 (m, 2H), 7.30-7.22 (m, 1H), 7.18 (d, J=7.3 Hz, 1H), 7.13 (s, 1H), 6.92 (d, J=8.1 Hz, 1H), 6.81-6.71 (m, 2H), 5.27 (s, 2H), 5.15-5.04 (m, 2H), 4.27 (s, 4H), 4.10 (q, J=7.1 Hz, 2H), 3.98-3.85 (m, 2H), 3.75-3.67 (m, 1H), 3.62 (dd, J=11.0, 6.6 Hz, 1H), 3.11 (t, J=5.5 Hz, 1H), 2.24 (s, 3H), 1.35 (t, J=7.3 Hz, 3H).

Examples 1006 to 1011 were prepared in substantially the same manner as Example 1005, using the appropriate amine for the reductive amination.

### Intermediate: (5-Bromopyridin-3-yl)methanol

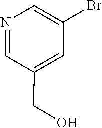

To a cold (0° C. ice bath) solution of 5-bromonicotinic acid (50 g, 248 mmol) in anhydrous THF (1000 mL), under nitrogen, was added N-methylmorpholine (27.2 mL, 248 mmol) and then ethyl chloroformate (23.67 mL, 248 mmol). After stirring for 20 minutes, sodium borohydride (28.1 g, 743 mmol) was added portionwise. The mixture was cooled (−78° C. dry ice acetone bath) and methanol (400 mL) was added over 90 minutes. The temperature was then allowed to rise to room temperature and stirring was continued for 24 hours. The reaction was concentrated under reduced pressure to a gummy semi-solid. The residue was suspended in dichloromethane (400 mL) and silica gel was added (˜100 g) to form a slurry. This was applied to the top of a short bed of silica gel and eluted with 10% methanol in dichloromethane. The filtrate was concentrated to a mobile yellow oil. The crude oil was purified by flash column chromatography (7.5 cm d×13 cm h) eluting with a step gradient from 0-5% methanol in dichloromethane. Product fractions were pooled and concentrated under reduced pressure, affording the product (24.5 g, 130 mmol, 52.6% yield) as a yellow mobile oil. LCMS (Condition ACN-TFA, ES+) M+H=188.0, 0.54 minutes, calculated exact mass=186.96. ¹H NMR (400 MHz, CDCl₃) δ: 8.59 (d, J=2.3 Hz, 1H), 8.49 (d, J=1.5 Hz, 1H), 7.93-7.87 (m, 1H), 4.74 (s, 2H).

### Intermediate: Methyl 3-((5-(hydroxymethyl)pyridin-3-yl)thio)propanoate

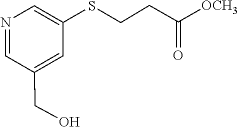 [SMILES: COC(=O)CCSc1cncc(CO)c1]

A mixture of (5-bromopyridin-3-yl)methanol (0.772 g, 4.11 mmol), methyl 3-mercaptopropanoate (0.500 mL, 4.52 mmol), Hunig's base (1.434 mL, 8.21 mmol), Pd₂(dba)₃ (0.113 g, 0.123 mmol) and Xantphos (0.143 g, 0.246 mmol) in dry dioxane (30 mL) was nitrogen sparged for 5 minutes, then heated (110° C. oil bath) for 16 hours. The reaction was cooled, then filtered, and the filter cake was washed with ethyl acetate. The filtrate was concentrated and purified (40 g SiO₂, methanol in dichloromethane 0-10% over 20 column volumes; two purifications were required), affording the product as a yellow oil. LCMS (Condition MeOH-AA, ES+) M+H=228.1, 1.58 minutes, calculated exact mass=227.06. ¹H NMR (400 MHz, CDCl₃) δ: 8.52 (d, J=2.2 Hz, 1H), 8.43 (d, J=1.7 Hz, 1H), 7.74 (t, J=2.0 Hz, 1H), 4.74 (s, 2H), 3.70 (s, 3H), 3.21 (t, J=7.3 Hz, 2H), 2.65 (t, J=7.3 Hz, 2H).

### Intermediate: Methyl 3-((5-(chloromethyl)pyridin-3-yl)thio)propanoate hydrochloride

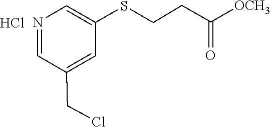 [SMILES: Cl ; COC(=O)CCSc1cncc(CCl)c1]

A solution of methyl 3-((5-(hydroxymethyl)pyridin-3-yl)thio)propanoate (0.109 g, 0.480 mmol) in dry dichloromethane (1.5 mL) was treated with thionyl chloride (0.210 mL, 2.88 mmol), stirred for 1 hour, and then concentrated to afford the product as a white solid. ¹H NMR (400 MHz, CDCl₃) δ: 8.62 (br. s., 1H), 8.57 (br. s., 1H), 8.27 (s, 1H), 4.72 (s, 2H), 3.75 (s, 3H), 3.40 (t, J=6.1 Hz, 2H), 2.78 (t, J=6.1 Hz, 2H).

### Intermediate: Methyl 3-((5-((4-chloro-5-((3-(2,3-dihydrobenzo[b][1,4]dioxin-6-yl)-2-methylbenzyl)oxy)-2-formylphenoxy)methyl)pyridin-3-yl)thio)propanoate

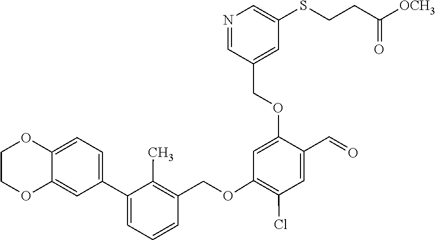 [SMILES: COC(=O)CCSc1cncc(COc2cc(OCc3cccc(-c4ccc5c(c4)OCCO5)c3C)c(Cl)cc2C=O)c1]

A solution of 5-chloro-4-((3-(2,3-dihydrobenzo[b][1,4]dioxin-6-yl)-2-methylbenzyl)oxy)-2-hydroxybenzaldehyde (0.140 g, 0.341 mmol) and methyl 3-((5-(chloromethyl)pyridin-3-yl)thio)propanoate hydrochloride (0.135 g, 0.480 mmol) in dry N,N-dimethylformamide (5 mL) was treated with cesium carbonate (0.33 g, 1.02 mmol) and sodium iodide (5 mg, 0.034 mmol). The mixture was stirred with heating (70° C. oil bath) for 1 hour, then cooled, diluted with ethyl acetate (30 mL), and washed with saturated aqueous sodium bicarbonate (10 mL), water, and brine. The organic layer was dried over sodium sulfate, filtered, and concentrated under reduced pressure. The residue was purified by silica gel column (24 g SiO₂, 0-70% ethyl acetate in hexanes), to afford the product (0.121 g, 0.195 mmol, 57.3% yield) as a glassy semi-solid. LCMS (Condition ACN-AA, ES+) M+H=620.0, 2.37 minutes, calculated exact mass=619.14. ¹H NMR (400 MHz, CDCl₃) δ: 10.31 (s, 1H), 8.60 (d, J=2.2 Hz, 1H), 8.52 (d, J=1.7 Hz, 1H), 7.92 (s, 1H), 7.82 (s, 1H), 7.44-7.40 (m, 1H), 6.93 (d, J=8.3 Hz, 1H), 6.84 (d, J=2.0 Hz, 1H), 6.82-6.75 (m, 1H), 6.67 (s, 1H), 5.23 (s, 2H), 5.18 (s, 2H), 4.32 (s, 4H), 3.70 (s, 3H), 3.23 (t, J=7.2 Hz, 2H), 2.66 (t, J=7.2 Hz, 2H), 2.30 (s, 3H) Note: two aromatic protons were obscured by CDCl₃ solvent peak.

### Example 1006: (S)-2-((5-Chloro-4-((3-(2,3-dihydrobenzo[b][1,4]dioxin-6-yl)-2-methylbenzyl)oxy)-2-((5-((3-methoxy-3-oxopropyl)thio)pyridin-3-yl)methoxy)benzyl)amino)-3-hydroxy-2-methylpropanoic acid

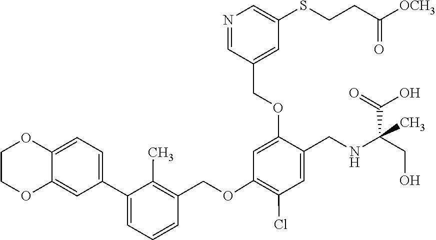 [SMILES: COC(=O)CCSc1cncc(COc2cc(OCc3cccc(-c4ccc5c(c4)OCCO5)c3C)c(Cl)cc2CN[C@@](C)(CO)C(=O)O)c1]

The yield of the product was 46.9 mg, and its estimated purity by LCMS analysis was 96% (LCMS Condition ACN-AA, ES+) M+H=723.3, 1.76 minutes, calculated exact mass=722.21. ¹H NMR (500 MHz, DMSO-d₆) δ: 8.56 (s, 1H), 8.47 (s, 1H), 7.98 (s, 1H), 7.54 (s, 1H), 7.41 (d, J=7.3 Hz, 1H), 7.27-7.20 (m, 1H), 7.16 (d, J=7.3 Hz, 1H), 7.11 (s, 1H), 6.92 (d, J=8.1 Hz, 1H), 6.77-6.70 (m, 2H), 5.28 (s, 2H), 5.25 (s, 2H), 4.26 (s, 4H), 4.00 (s, 2H), 3.55 (s, 3H), 3.23 (t, J=6.8 Hz, 2H), 2.62 (t, J=7.0 Hz, 2H), 2.22 (s, 3H), 1.23 (s, 3H): Note: some product peaks were obscured by solvent.

### Example 1007: (S)-1-(5-Chloro-4-((3-(2,3-dihydrobenzo[b][1,4]dioxin-6-yl)-2-methylbenzyl)oxy)-2-((5-((3-methoxy-3-oxopropyl)thio)pyridin-3-yl)methoxy)benzyl)piperidine-2-carboxylic acid

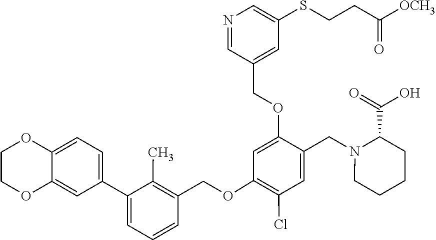 [SMILES: COC(=O)CCSc1cncc(COc2cc(OCc3cccc(-c4ccc5c(c4)OCCO5)c3C)c(Cl)cc2CN2CCCC[C@H]2C(=O)O)c1]

The yield of the product was 34.0 mg, and its estimated purity by LCMS analysis was 96% (LCMS Condition ACN-AA, ES+) M+H=733.3, 1.90 minutes, calculated exact mass=732.23. ¹H NMR (500 MHz, DMSO-d₆) δ: 8.53 (s, 1H), 8.50 (s, 1H), 7.91 (s, 1H), 7.50-7.40 (m, 2H), 7.29-7.21 (m, 1H), 7.18 (d, J=7.7 Hz, 1H), 7.11 (s, 1H), 6.92 (d, J=8.4 Hz, 1H), 6.81-6.72 (m, 2H), 5.25 (s, 2H), 5.23 (s, 2H), 4.28 (s, 4H), 3.76 (d, J=13.6 Hz, 1H), 3.62 (d, J=13.9 Hz, 1H), 3.56 (s, 3H), 3.23 (t, J=7.0 Hz, 2H), 3.10 (d, J=3.7 Hz, 1H), 2.89 (br. s., 1H), 2.63 (t, J=6.8 Hz, 2H), 2.32-2.17 (m, 4H), 1.79 (br. s., 1H), 1.69 (d, J=9.5 Hz, 1H), 1.48 (br. s., 3H), 1.34 (br. s., 1H).

### Intermediate: 2-(4-(Hydroxymethyl)-1H-pyrazol-1-yl)acetonitrile

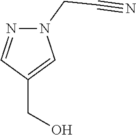

A solution of (1H-pyrazol-4-yl)methanol (0.070 g, 0.714 mmol) and 2-bromoacetonitrile (0.052 mL, 0.749 mmol) in dry acetonitrile (5.0 mL) was treated with potassium carbonate (0.197 g, 1.427 mmol). The reaction was stirred for 18 hours, then treated with additional 2-bromoacetonitrile (0.052 mL, 0.749 mmol), heated (55° C. oil bath) and stirred for 72 hours. The mixture was diluted with acetonitrile and filtered through celite. The filtrate was concentrated under reduced pressure and the crude residue was purified by flash column chromatography, eluting with 0-10% methanol in dichloromethane to afford the product. LCMS (Condition ACN-TFA, ES+) M+H=138.0, 0.039 minutes, calculated exact mass=137.06. ¹H NMR (400 MHz, d6-Acetone) δ: 7.72 (s, 1H), 7.49 (s, 1H), 5.35 (s, 2H), 4.50 (d, J=5.3 Hz, 2H), 3.94 (t, J=5.6 Hz, 1H).

### Intermediate: 2-(4-(chloromethyl)-1H-pyrazol-1-yl)acetonitrile

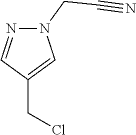

A flask charged with 2-(4-(hydroxymethyl)-1H-pyrazol-1-yl)acetonitrile (0.070 g, 0.510 mmol) and dichloromethane (2 mL) was treated with thionyl chloride (0.224 mL, 3.06 mmol), the reaction was stirred for 16 hours, then concentrated under reduced pressure, to afford the product., which was used immediately in the following reaction. ¹H NMR (400 MHz, d6-Acetone) δ: 7.94 (s, 1H), 7.63 (s, 1H), 5.41 (s, 2H), 4.68 (s, 2H).

### Intermediate: 2-(4-((4-Chloro-5-((3-(2,3-dihydrobenzo[b][1,4]dioxin-6-yl)-2-methylbenzyl)oxy)-2-formylphenoxy)methyl)-1H-pyrazol-1-yl)acetonitrile

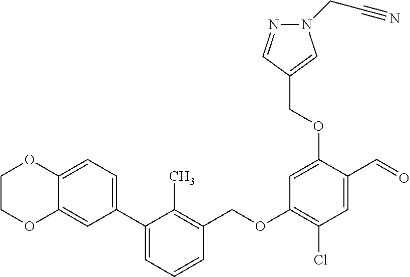 [SMILES: Cc1c(COc2cc(OCc3cnn(CC#N)c3)c(C=O)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-hydroxybenzaldehyde (0.100 g, 0.243 mmol) and 2-(4-(chloromethyl)-1H-pyrazol-1-yl)acetonitrile (0.079 g, 0.510 mmol) in dry N,N-dimethylformamide (3.0 mL) was treated with cesium carbonate (0.238 g, 0.730 mmol) and sodium iodide (4 mg, 0.024 mmol). The mixture was heated (75° C. oil bath) for 5 hours, then cooled, diluted with ethyl acetate, and washed with water then brine. The organic layer was dried over sodium sulfate, filtered, and concentrated under reduced pressure. The residue was purified by biotage (RediSep 24 g SiO₂, 0% (3 CV), 0-100% (25 CV), 100% (5 CV), ethyl acetate in hexanes). Product fractions were pooled and concentrated under reduced pressure, affording the product (0.052 g, 0.098 mmol, 40.3% yield) as a yellow viscous oil. The material was used as-is without further purification. LCMS (Condition ACN-TFA, ES+) M+H=530.2, 1.12 minutes, calculated exact mass=529.14. ¹H NMR (400 MHz, CDCl₃) δ: 10.24 (s, 1H), 7.89 (s, 1H), 7.62 (d, J=12.0 Hz, 2H), 7.44 (t, J=4.6 Hz, 1H), 7.28 (d, J=1.0 Hz, 1H), 6.93 (d, J=8.3 Hz, 1H), 6.83 (d, J=2.0 Hz, 1H), 6.78 (dd, J=8.3, 2.0 Hz, 1H), 6.66 (s, 1H), 5.25 (s, 2H), 5.08 (s, 4H), 4.32 (s, 4H), 2.31 (s, 3H); Note: one aromatic signal was obscured by CDCl₃.

### Example 1008: (S)-2-((5-chloro-2-((1-(cyanomethyl)-1H-pyrazol-4-yl)methoxy)-4-((3-(2,3-dihydrobenzo[b][1,4]dioxin-6-yl)-2-methylbenzyl)oxy)benzyl)amino)-3-hydroxy-2-methylpropanoic acid

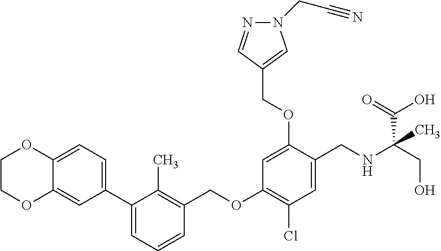 [SMILES: Cc1c(COc2cc(OCc3cnn(CC#N)c3)c(CN[C@@](C)(CO)C(=O)O)cc2Cl)cccc1-c1ccc2c(c1)OCCO2]

The yield of the product was 23.5 mg, and its estimated purity by LCMS analysis was 100% (LCMS Condition ACN-AA, ES+) M+H=633.3, 1.65 minutes, calculated exact mass=632.20. ¹H NMR (500 MHz, DMSO-d₆) δ: 8.06 (s, 1H), 7.75 (s, 1H), 7.57-7.43 (m, 2H), 7.26 (t, J=7.3 Hz, 1H), 7.18 (d, J=7.7 Hz, 1H), 7.14 (s, 1H), 6.92 (d, J=8.1 Hz, 1H), 6.80-6.70 (m, 2H), 5.47 (s, 2H), 5.28 (s, 2H), 5.15 (s, 2H), 4.27 (s, 4H), 3.91 (s, 2H), 3.64 (d, J=11.4 Hz, 1H), 3.52 (d, J=11.4 Hz, 1H), 2.24 (s, 3H), 1.23 (s, 3H).

### Intermediate: (1H-Pyrazol-4-yl)methanol

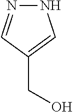

A solution of ethyl 1H-pyrazole-4-carboxylate (1.431 g, 10.21 mmol) in dry THF (25.0 mL) was added dropwise to a cold (0° C. ice bath) suspension of lithium aluminum hydride (0.568 g, 14.97 mmol) in dry THF (100 mL). The reaction was stirred with cooling for 10 minutes, then allowed to warm to room temperature and stirred for 16 hours. The reaction was quenched by dropwise addition of water (0.60 mL), then 15% sodium hydroxide (0.60 mL) and finally water (1.70 mL). The mixture was diluted with ethyl acetate (30 mL) and stirred for 2 hours. The solids were removed by filtration, washing with several portions of ethyl acetate, and the filtrate was concentrated under reduced pressure. The resulting oil was dried under vacuum pump, resulting in a crystalline solid. The solids were recrystallized from dichloromethane, affording the product (0.128 g, 1.305 mmol, 12.78% yield) after the mother liquor was decanted and the crystals were dried under vacuum pump. ¹H NMR (400 MHz, CD₃OD) δ: 7.58 (br. s., 2H), 6.93 (d, J=0.5 Hz, 1H), 4.54 (s, 2H).

### Intermediate: tert-Butyl 2-(4-(hydroxymethyl)-1H-pyrazol-1-yl)acetate

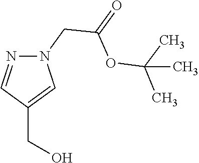

A solution of (1H-pyrazol-4-yl)methanol (0.128 g, 1.305 mmol) and tert-butyl 2-bromoacetate (0.289 mL, 1.957 mmol) in dry acetonitrile (6.0 mL) was treated with potassium carbonate (0.361 g, 2.61 mmol). The reaction was stirred for 72 hours, whereupon the mixture was diluted with acetonitrile and filtered through celite. The filtrate was concentrated under reduced pressure. The residue was purified by biotage (RediSep 40 g SiO₂, 0% (3 CV), 0-10% (10 CV), 10% (2 CV), methanol in dichloromethane). Product fractions were pooled and concentrated under reduced pressure, affording the product (0.271 g, 1.277 mmol, 98% yield) as a clear viscous oil. LCMS (Condition ACN-TFA, ES+) M-tBu+H=157.1, 0.63 minutes, calculated exact mass=212.12. ¹H NMR (400 MHz, CDCl₃) δ: 7.45 (s, 1H), 7.39 (s, 1H), 4.73 (s, 2H), 4.50 (s, 2H), 1.44 (s, 9H).

### Intermediate: tert-butyl 2-(4-(chloromethyl)-1H-pyrazol-1-yl)acetate

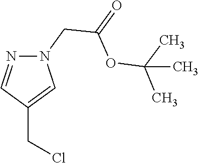

A cold (0° C. ice bath) solution of tert-butyl 2-(4-(hydroxymethyl)-1H-pyrazol-1-yl)acetate (0.102 g, 0.481 mmol) in dry dichloromethane (2.4 mL) was treated with Hunig's base (0.252 mL, 1.442 mmol) followed by methanesulfonyl chloride (0.075 mL, 0.961 mmol) dropwise. The ice bath was removed and the mixture was stirred for 6 hours, after which the reaction was diluted with additional dichloromethane and washed with water. The organic layer was dried over sodium sulfate, filtered, and concentrated under reduced pressure affording the product which was used immediately in the following step. ¹H NMR (400 MHz, CDCl₃) δ: 7.58 (s, 1H), 7.53 (s, 1H), 4.79 (s, 2H), 4.57 (s, 2H), 1.48 (s, 9H).

### Intermediate: tert-Butyl 2-(4-((4-chloro-5-((3-(2,3-dihydrobenzo[b][1,4]dioxin-6-yl)-2-methylbenzyl)oxy)-2-formylphenoxy)methyl)-1H-pyrazol-1-yl)acetate

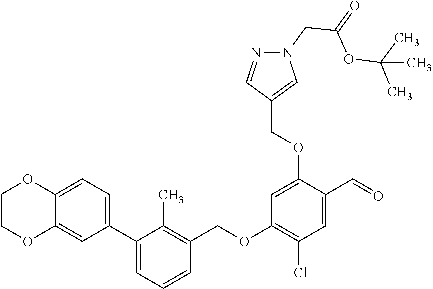 [SMILES: Cc1c(COc2cc(OCc3cnn(CC(=O)OC(C)(C)C)c3)c(C=O)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-hydroxybenzaldehyde (0.126 g, 0.307 mmol) and tert-butyl 2-(4-(chloromethyl)-1H-pyrazol-1-yl)acetate (0.111 g, 0.481 mmol) in dry N,N-dimethylformamide (3.0 mL) was treated with cesium carbonate (0.310 g, 0.951 mmol) and sodium iodide (5 mg, 0.033 mmol). The mixture was heated (70° C. oil bath) for 90 minutes, then cooled and stirred for 16 hours. The reaction mixture was diluted with ethyl acetate (20 mL) and washed with water (2×50 mL) and brine (50 mL), then dried over sodium sulfate, filtered, and concentrated under reduced pressure to afford the crude product as a glassy brown solid after drying twice from diethyl ether. The residue was purified by biotage (RediSep 12 g SiO₂, 0% (3 CV), 0-25% (15 CV), 25% (2 CV), methanol in dichloromethane). Product fractions were pooled and concentrated under reduced pressure, affording the product (0.134 g, 0.221 mmol, 72.2% yield) as a brown viscous oil. LCMS (Condition ACN-TFA, ES+) M+H=605.3, 1.18 minutes, calculated exact mass=604.20. ¹H NMR (400 MHz, CDCl₃) δ: 10.26 (s, 1H), 7.88 (s, 1H), 7.59 (s, 1H), 7.52 (s, 1H), 7.45-7.42 (m, 1H), 7.26 (s, 1H), 6.92 (d, J=8.3 Hz, 1H), 6.83 (d, J=2.0 Hz, 1H), 6.78 (dd, J=8.2, 2.1 Hz, 1H), 6.69 (s, 1H), 5.23 (s, 2H), 5.11 (s, 2H), 4.81 (s, 2H), 4.32 (s, 4H), 2.30 (s, 3H), 1.47 (s, 9H).

### Example 1009: (S)-2-((2-((1-(2-(tert-Butoxy)-2-oxoethyl)-1H-pyrazol-4-yl)methoxy)-5-chloro-4-((3-(2,3-dihydrobenzo[b][1,4]dioxin-6-yl)-2-methylbenzyl)oxy)benzyl)amino)-3-hydroxy-2-methylpropanoic acid

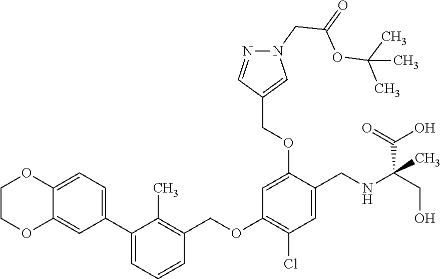 [SMILES: Cc1c(COc2cc(OCc3cnn(CC(=O)OC(C)(C)C)c3)c(CN[C@@](C)(CO)C(=O)O)cc2Cl)cccc1-c1ccc2c(c1)OCCO2]

The yield of the product was 16.6 mg, and its estimated purity by LCMS analysis was 98% (LCMS Condition ACN-AA, ES+) M+H=708.3, 1.84 minutes, calculated exact mass=707.26. ¹H NMR (500 MHz, DMSO-d₆) δ: 7.90 (s, 1H), 7.61 (s, 1H), 7.53-7.44 (m, 2H), 7.30-7.23 (m, 1H), 7.18 (d, J=7.7 Hz, 1H), 7.13 (s, 1H), 6.92 (d, J=8.1 Hz, 1H), 6.80-6.70 (m, 2H), 5.27 (s, 2H), 5.15 (s, 2H), 4.91 (s, 2H), 4.28 (s, 4H), 3.87 (s, 2H), 3.60 (d, J=11.0 Hz, 1H), 3.51 (d, J=11.0 Hz, 1H), 2.24 (s, 3H), 1.39 (s, 9H), 1.22 (s, 3H).

### Intermediate: (5-(Benzylthio)pyridin-3-yl)methanol

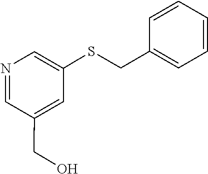 [SMILES: OCc1cncc(SCc2ccccc2)c1]

A mixture of (5-bromopyridin-3-yl)methanol (1.078 g, 5.73 mmol), benzyl mercaptan (0.712 mL, 6.02 mmol), Hunig's base (2.003 mL, 11.47 mmol), Pd₂(dba)₃ (0.158 g, 0.172 mmol) and Xantphos (0.199 g, 0.344 mmol) in dry dioxane (25 mL) was nitrogen sparged for 5 minutes, then heated (110° C. oil bath) for 16 hours. The reaction was cooled, then diluted with ethyl acetate. The solids were removed by filtration, washing with ethyl acetate. The filtrate developed crystals upon standing. The crystals were collected by vacuum filtration, washing with ethyl acetate. The filtrate was concentrated under reduced pressure and the residue was purified by biotage (RediSep 40 g SiO₂, 0% (3 CV), 0-100% (15 CV), 100% (4 CV), ethyl acetate in hexanes). Product fractions were pooled and concentrated under reduced pressure, affording the product (1.289 g, 5.57 mmol, 97% yield) as a pale yellow viscous oil. LCMS (Condition ACN-TFA, ES+) M+H=232.1, 0.64 minutes, calculated exact mass=231.07. ¹H NMR (400 MHz, CDCl₃) δ: 8.39 (d, J=2.3 Hz, 1H), 8.33 (d, J=2.0 Hz, 1H), 7.59 (t, J=2.0 Hz, 1H), 7.32-7.22 (m, 5H), 4.64 (s, 2H), 4.11 (s, 2H).

### Intermediate: 3-(Benzylthio)-5-(chloromethyl)pyridine hydrochloride

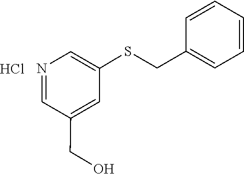 [SMILES: Cl ; OCc1cncc(SCc2ccccc2)c1]

A solution of (5-(benzylthio)pyridin-3-yl)methanol (0.336 g, 1.453 mmol) in dry dichloromethane (10.0 mL) was treated with thionyl chloride (0.70 mL, 9.59 mmol), and the mixture was stirred for 2 hours. The reaction was concentrated by rotary evaporator to a pale yellow powdery solid, which was used immediately in the following step. ¹H NMR (400 MHz, CDCl₃) δ: 8.55 (d, J=1.3 Hz, 1H), 8.48 (d, J=2.0 Hz, 1H), 8.10 (s, 1H), 7.40-7.31 (m, 5H), 4.63 (s, 2H), 4.30 (s, 2H).

### Intermediate: 2-((5-(Benzylthio)pyridin-3-yl)methoxy)-5-chloro-4-((3-(2,3-dihydrobenzo[b][1,4]dioxin-6-yl)-2-methylbenzyl)oxy)benzaldehyde

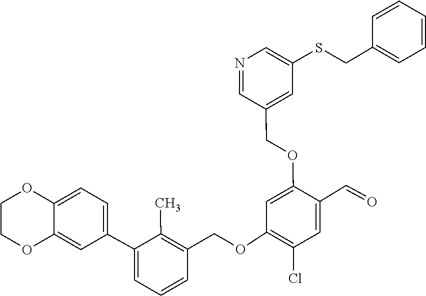 [SMILES: Cc1c(COc2cc(OCc3cncc(SCc4ccccc4)c3)c(C=O)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-hydroxybenzaldehyde (0.337 g, 0.820 mmol) and 3-(benzylthio)-5-(chloromethyl)pyridine hydrochloride (0.416 g, 1.453 mmol) in N,N-dimethylformamide (6 mL) was treated with cesium carbonate (0.652 g, 2.001 mmol) and sodium iodide (0.022 g, 0.147 mmol), and the mixture was then stirred with heating (75° C. oil bath) for 2 hours, then cooled. The reaction was diluted with ethyl acetate (25 mL), and washed with water (3×25 mL) and brine. The organic layer was dried over sodium sulfate, filtered, and concentrated under reduced pressure. The residue was triturated with diethyl ether, then partially dissolved in ethyl acetate (˜5 mL) followed by dilution with a large volume of diethyl ether (˜100 mL). The solids were collected by vacuum filtration then dried under vacuum, affording the product (0.396 g, 0.634 mmol, 79% yield) as a tan powdery solid, and which was used without further purification in the following step. LCMS (Condition ACN-TFA, ES+) M+H=624.2, 1.28 minutes, calculated exact mass=623.15. ¹H NMR (400 MHz, CDCl₃) δ: 10.25 (s, 1H), 8.54 (d, J=2.3 Hz, 1H), 8.46 (d, J=2.0 Hz, 1H), 7.92 (s, 1H), 7.63 (t, J=2.0 Hz, 1H), 7.43-7.37 (m, 1H), 7.26-7.21 (m, 5H), 6.93 (d, J=8.3 Hz, 1H), 6.84 (d, J=2.0 Hz, 1H), 6.79 (dd, J=8.2, 2.1 Hz, 1H), 6.60 (s, 1H), 5.19 (s, 2H), 5.11 (s, 2H), 4.32 (s, 4H), 4.14 (s, 2H), 2.29 (s, 3H), Note: some aromatic resonances partially obscured by CDCl₃ solvent peak.

### Example 1010: (S)-1-(2-((5-(Benzylthio)pyridin-3-yl)methoxy)-5-chloro-4-((3-(2,3-dihydrobenzo [b][1,4]dioxin-6-yl)-2-methylbenzyl)oxy)benzyl)piperidine-2-carboxylic acid

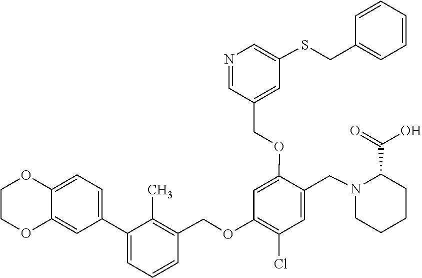 [SMILES: Cc1c(COc2cc(OCc3cncc(SCc4ccccc4)c3)c(CN3CCCC[C@H]3C(=O)O)cc2Cl)cccc1-c1ccc2c(c1)OCCO2]

The yield of the product was 4.9 mg, and its estimated purity by LCMS analysis was 95% (LCMS Condition ACN-AA, ES+) M+H=737.3, 2.03 minutes, calculated exact mass=736.24. ¹H NMR (500 MHz, DMSO-d₆) δ: 8.50 (s, 1H), 8.44 (s, 1H), 7.90 (s, 1H), 7.49-7.42 (m, 2H), 7.30-7.15 (m, 7H), 7.09 (s, 1H), 6.92 (d, J=8.1 Hz, 1H), 6.81-6.71 (m, 2H), 5.22 (d, J=6.6 Hz, 4H), 4.30-4.26 (m, 7H), 3.89 (s, 2H), 3.80 (d, J=13.9 Hz, 1H), 3.65 (d, J=13.9 Hz, 1H), 2.31 (d, J=4.8 Hz, 1H), 2.23 (s, 5H), 1.81 (br. s., 1H), 1.69 (d, J=10.3 Hz, 1H), 1.33 (br. s., 1H).

### Intermediate: 2-(4-(Chloromethyl)-1H-pyrazol-1-yl)pyridine hydrochloride

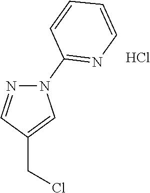 [SMILES: ClCc1cnn(-c2ccccn2)c1 ; Cl]

A suspension of (1-(pyridin-2-yl)-1H-pyrazol-4-yl)methanol (0.151 g, 0.862 mmol) in dry dichloromethane (5 mL) was treated with thionyl chloride (0.377 mL, 5.17 mmol), and the mixture was stirred for 2 hours. The solvent was removed by rotary evaporator to afford a pale yellow solid, which was used immediately in the following step. ¹H NMR (400 MHz, DMSO-d₆) δ: 8.71 (d, J=0.5 Hz, 1H), 8.52-8.45 (m, 1H), 8.04-7.97 (m, 1H), 7.94-7.89 (m, 2H), 7.37 (ddd, J=7.3, 4.8, 1.1 Hz, 1H), 5.76 (s, 2H).

### Intermediate: 5-Chloro-4-((3-(2,3-dihydrobenzo[b][1,4]dioxin-6-yl)-2-methylbenzyl)oxy)-2-((1-(pyridin-2-yl)-1H-pyrazol-4-yl)methoxy)benzaldehyde

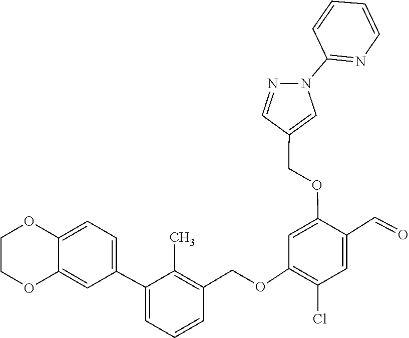 [SMILES: Cc1c(COc2cc(OCc3cnn(-c4ccccn4)c3)c(C=O)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-hydroxybenzaldehyde (0.142 g, 0.345 mmol) and 2-(4-(chloromethyl)-1H-pyrazol-1-yl)pyridine hydrochloride (0.167 g, 0.862 mmol) in dry N,N-dimethylformamide (3.0 mL) was treated with cesium carbonate (0.393 g, 1.207 mmol) and sodium iodide (5.17 mg, 0.034 mmol). The mixture was heated (75° C. oil bath) for 3 hours, and then cooled to room temperature. The reaction mixture was diluted with ethyl acetate and washed with water (3×10 mL) and then brine. The organic layer was dried over sodium sulfate, filtered, and concentrated under reduced pressure, affording the product (0.175 g, 0.308 mmol, 89% yield) as a yellow soft solid. LCMS (Condition ACN-TFA, ES+) M+H=568.2, 1.27 minutes, calculated exact mass=567.16. ¹H NMR (400 MHz, CDCl₃) δ: 10.30 (s, 1H), 8.67 (s, 1H), 8.41 (ddd, J=4.8, 1.8, 0.8 Hz, 1H), 8.01-7.94 (m, 1H), 7.90 (s, 1H), 7.88-7.81 (m, 2H), 7.78 (s, 1H), 7.44 (dd, J=6.0, 3.0 Hz, 1H), 7.23 (ddd, J=7.3, 4.8, 1.0 Hz, 2H), 6.92 (d, J=8.3 Hz, 1H), 6.84 (d, J=2.0 Hz, 1H), 6.79 (dd, J=8.3, 2.0 Hz, 1H), 6.72 (s, 1H), 5.25 (s, 2H), 5.18 (s, 2H), 4.32 (s, 4H), 2.30 (s, 3H).

### Example 1011: (S)-2-((5-Chloro-4-((3-(2,3-dihydrobenzo[b][1,4]dioxin-6-yl)-2-methylbenzyl)oxy)-2-((1-(pyridin-2-yl)-1H-pyrazol-4-yl)methoxy)benzyl)amino)-3-hydroxy-2-methylpropanoic acid

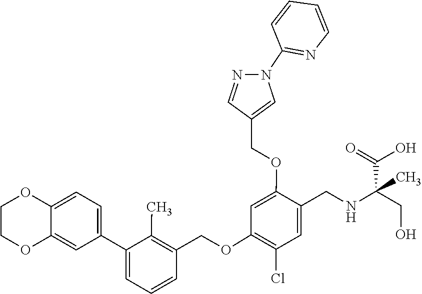 [SMILES: Cc1c(COc2cc(OCc3cnn(-c4ccccn4)c3)c(CN[C@@](C)(CO)C(=O)O)cc2Cl)cccc1-c1ccc2c(c1)OCCO2]

The yield of the product was 34.7 mg, and its estimated purity by LCMS analysis was 98% (Condition ACN-AA, ES+) M+H=671.10, 1.88 minutes, calculated exact mass=670.22. ¹H NMR (500 MHz, DMSO-d₆) δ: 8.79 (s, 1H), 8.45 (d, J=4.0 Hz, 1H), 8.02 (s, 1H), 8.01-7.95 (m, 1H), 7.95-7.89 (m, 1H), 7.51 (s, 1H), 7.47 (d, J=7.3 Hz, 1H), 7.38-7.32 (m, 1H), 7.25-7.21 (m, 1H), 7.19 (s, 1H), 7.16 (d, J=7.7 Hz, 1H), 6.92 (d, J=8.1 Hz, 1H), 6.77 (s, 1H), 6.74 (d, J=8.4 Hz, 1H), 5.30 (s, 2H), 5.26 (s, 2H), 4.28 (s, 4H), 3.92 (s, 2H), 3.63 (d, J=11.4 Hz, 1H), 3.53 (d, J=11.4 Hz, 1H), 2.23 (s, 3H), 1.24 (s, 3H).

### Intermediate: (S)-1-(2-((5-((2-Carboxyethyl)thio)pyridin-3-yl)methoxy)-5-chloro-4-((3-(2,3-dihydrobenzo[b][1,4]dioxin-6-yl)-2-methylbenzyl)oxy)benzyl)piperidine-2-carboxylic acid

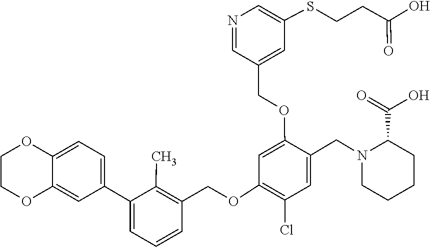 [SMILES: Cc1c(COc2cc(OCc3cncc(SCCC(=O)O)c3)c(CN3CCCC[C@H]3C(=O)O)cc2Cl)cccc1-c1ccc2c(c1)OCCO2]

A solution of (S)-1-(5-chloro-4-((3-(2,3-dihydrobenzo[b][1,4]dioxin-6-yl)-2-methylbenzyl)oxy)-2-((5-((3-methoxy-3-oxopropyl)thio)pyridin-3-yl)methoxy)benzyl) piperidine-2-carboxylic acid (0.030 g, 0.041 mmol, Example 1008) in methanol (0.2 mL) and THF (0.2 mL) was treated with 1.0 M aqueous sodium hydroxide (0.2 mL, 0.200 mmol), and the mixture was stirred for 90 minutes. The mixture was treated with trifluoroacetic acid (0.015 mL, 0.200 mmol), then concentrated to remove organic solvents. The residue was dried as an azeoptrope from acetonitrile three times by rotary evaporator. The residue was used directly in the following experiment. LCMS (Condition ACN-TFA, ES+) M+H=719.2, 1.07 minutes, calculated exact mass=718.21.

### Example 1012: (S)-1-(2-((5-((2-Carboxyethyl)sulfonyl)pyridin-3-yl)methoxy)-5-chloro-4-((3-(2,3-dihydrobenzo[b]1[1,4]dioxin-6-yl)-2-methylbenzyl)oxy)benzyl)piperidine-2-carboxylic acid

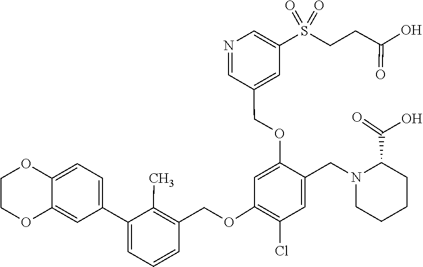 [SMILES: Cc1c(COc2cc(OCc3cncc(S(=O)(=O)CCC(=O)O)c3)c(CN3CCCC[C@H]3C(=O)O)cc2Cl)cccc1-c1ccc2c(c1)OCCO2]

A suspension of (S)-1-(2-((5-((2-carboxyethyl)thio)pyridin-3-yl)methoxy)-5-chloro-4-((3-(2,3-dihydrobenzo[b]1[1,4]dioxin-6-yl)-2-methylbenzyl)oxy)benzyl) piperidine-2-carboxylic acid (0.029 g, 0.041 mmol) in dry dichloromethane (0.5 mL) was treated with mCPBA (0.030 g, 0.123 mmol), resulting in dissolution of all solids within several minutes. The reaction was stirred for 3 hours, then treated with saturated aqueous sodium sulfite (0.5 mL, 1.071 mmol), and the mixture was stirred for 16 hour. The mixture was diluted with dichloromethane (2 mL) and water (2 mL) and the layers were separated. The aqueous layer was extracted with dichloromethane (3×2 mL). The combined extracts were dried over sodium sulfate, filtered, and concentrated under reduced pressure, resulting in a solid upon standing. The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-50% B over 30 minutes, then a 5-minute hold at 100% B; Flow: 20 mL/min. Fractions containing the desired product were combined and dried via centrifugal evaporation, affording the product. The yield of the product was 0.4 mg, and its estimated purity by LCMS analysis was 97%. LCMS (Condition ACN-AA, ES+) M+H=751.1, 1.42 minutes, calculated exact mass=750.20.

### Example 1013: (S)-2-((2-((3-(4-(Carboxymethyl)thiazol-2-yl)benzyl)oxy)-5-chloro-4-((3-(2,3-dihydrobenzo[b][1,4]dioxin-6-yl)-2-methylbenzyl)oxy)benzyl)amino)-3-hydroxy-2-methylpropanoic acid

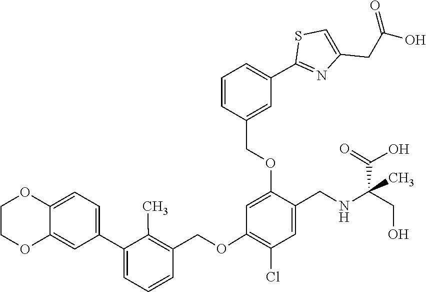 [SMILES: Cc1c(COc2cc(OCc3cccc(-c4nc(CC(=O)O)cs4)c3)c(CN[C@@](C)(CO)C(=O)O)cc2Cl)cccc1-c1ccc2c(c1)OCCO2]

A solution of (S)-2-((5-chloro-4-((3-(2,3-dihydrobenzo[b][1,4]dioxin-6-yl)-2-methylbenzyl)oxy)-2-((3-(4-(2-ethoxy-2-oxoethyl)thiazol-2-yl)benzyl)oxy)benzyl) amino)-3-hydroxy-2-methylpropanoic acid (0.066 g, 0.085 mmol) in methanol (0.427 mL) and THF (0.427 mL) was treated with 1.0 N sodium hydroxide (0.427 mL, 0.427 mmol). The mixture was stirred for 90 minutes. The reaction was quenched by addition of several drops of trifluoroacetic acid, then the solvent was removed by rotary evaporator. The residue was dried three times from methanol, then the crude material was purified via preparative LCMS with the following conditions: Column: XBridge C18, 19×200 mm, 5-μm particles; Mobile Phase A: 5:95 methanol: water with 10-mM ammonium acetate; Mobile Phase B: 95:5 methanol: water with 10-mM ammonium acetate; Gradient: 60-100% B over 20 minutes, then a 5-minute hold at 100% B; Flow: 20 mL/min. Fractions containing the desired product were combined and dried via centrifugal evaporation. The yield of the product was 57.7 mg, and its estimated purity by LCMS analysis was 99% (Condition MeOH-AA, ES+) M+H=745.3, 2.74 minutes, calculated exact mass=744.19. ¹H NMR (500 MHz, DMSO-d₆) δ: 8.12 (s, 1H), 7.86 (d, J=7.7 Hz, 1H), 7.67 (d, J=7.3 Hz, 1H), 7.58-7.48 (m, 3H), 7.42 (d, J=7.3 Hz, 1H), 7.27-7.19 (m, 1H), 7.19-7.12 (m, 2H), 6.92 (d, J=8.1 Hz, 1H), 6.78-6.68 (m, 2H), 5.34 (s, 2H), 5.25 (s, 2H), 4.27 (s, 4H), 4.02 (br. s., 2H), 3.76 (s, 2H), 3.68 (d, J=11.0 Hz, 1H), 2.20 (s, 3H), 1.26 (s, 3H).

Examples 1014 and 1015 were prepared in substantially the same manner as Example 1013.

### Example 1014: (S)-2-((2-((5-((2-Carboxyethyl)thio)pyridin-3-yl)methoxy)-5-chloro-4-((3-(2,3-dihydrobenzo[b][1,4]dioxin-6-yl)-2-methylbenzyl)oxy)benzyl)amino)-3-hydroxy-2-methylpropanoic acid

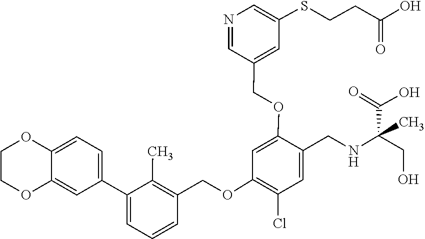 [SMILES: Cc1c(COc2cc(OCc3cncc(SCCC(=O)O)c3)c(CN[C@@](C)(CO)C(=O)O)cc2Cl)cccc1-c1ccc2c(c1)OCCO2]

The yield of the product was 2.3 mg, and its estimated purity by LCMS analysis was 96% (Condition MeOH-AA, ES+) M+H=709.3, 2.60 minutes, calculated exact mass=708.19. ¹H NMR (500 MHz, DMSO-d₆) δ: 8.54 (br. s., 1H), 8.51 (br. s., 1H), 7.99 (br. s., 1H), 7.56 (s, 1H), 7.48 (d, J=7.7 Hz, 1H), 7.31-7.23 (m, 1H), 7.22-7.14 (m, 2H), 6.93 (d, J=8.1 Hz, 1H), 6.81-6.68 (m, 2H), 5.31 (s, 2H), 5.26 (d, J=4.8 Hz, 2H), 4.28 (s, 4H), 4.11-3.95 (m, 2H), 3.70 (d, J=11.7 Hz, 1H), 3.58 (d, J=11.0 Hz, 1H), 3.53-3.46 (m, 2H), 3.20 (t, J=6.6 Hz, 2H), 2.64 (t, J=6.6 Hz, 2H), 2.25 (s, 3H), 1.26 (s, 3H).

### Example 1015: (S)-2-((2-((3-(4-(Carboxymethyl)oxazol-2-yl)benzyl)oxy)-5-chloro-4-((3-(2,3-dihydrobenzo[b][1,4]dioxin-6-yl)-2-methylbenzyl)oxy)benzyl)amino)-3-hydroxy-2-methylpropanoic acid

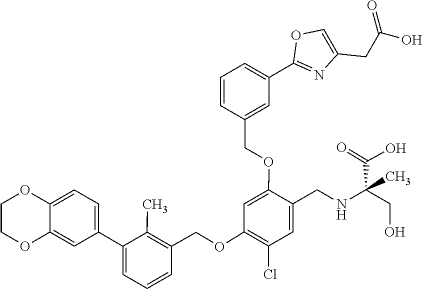 [SMILES: Cc1c(COc2cc(OCc3cccc(-c4nc(CC(=O)O)co4)c3)c(CN[C@@](C)(CO)C(=O)O)cc2Cl)cccc1-c1ccc2c(c1)OCCO2]

The yield of the product was 7.7 mg, and its estimated purity by LCMS analysis was 95% (Condition MeOH-AA, ES+) M+H=729.3, 2.65 minutes, calculated exact mass=728.21. ¹H NMR (500 MHz, DMSO-d₆) δ: 8.15 (s, 1H), 7.99 (s, 1H), 7.90 (d, J=8.1 Hz, 1H), 7.70 (d, J=8.1 Hz, 1H), 7.58-7.51 (m, 2H), 7.40 (d, J=7.3 Hz, 1H), 7.24-7.18 (m, 1H), 7.17-7.11 (m, 2H), 6.91 (d, J=8.1 Hz, 1H), 6.77-6.68 (m, 2H), 5.34 (s, 2H), 5.24 (s, 2H), 4.26 (s, 4H), 4.03 (s, 2H), 3.52 (s, 2H), 2.19 (s, 3H), 1.26 (s, 3H).

### Example 1016: (S)-2-((2-((1-(Carboxymethyl)-1H-pyrazol-4-yl)methoxy)-5-chloro-4-((3-(2,3-dihydrobenzo[b][1,4]dioxin-6-yl)-2-methylbenzyl)oxy)benzyl)amino)-3-hydroxy-2-methylpropanoic acid

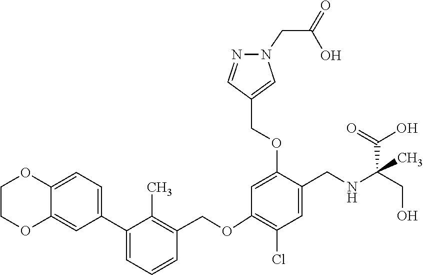 [SMILES: Cc1c(COc2cc(OCc3cnn(CC(=O)O)c3)c(CN[C@@](C)(CO)C(=O)O)cc2Cl)cccc1-c1ccc2c(c1)OCCO2]

A solution of(S)-2-((2-((1-(2-(tert-butoxy)-2-oxoethyl)-1H-pyrazol-4-yl)methoxy)-5-chloro-4-((3-(2,3-dihydrobenzo[b][1,4]dioxin-6-yl)-2-methylbenzyl)oxy) benzyl)amino)-3-hydroxy-2-methylpropanoic acid (0.015 g, 0.021 mmol) in dry dichloromethane (0.5 mL) was treated with trifluoroacetic acid (0.5 mL, 6.49 mmol), then stirred for 2 hours. The reaction was concentrated under reduced pressure and the residue was purified via preparative LCMS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 25-65% B over 30 minutes, then a 5-minute hold at 100% B; Flow: 20 mL/min. Fractions containing the desired product were combined and dried via centrifugal evaporation. The yield of the product was 5.0 mg, and its estimated purity by LCMS analysis was 98% (Condition MeOH-AA, ES+) M+H=652.2, 2.56 minutes, calculated exact mass=651.20. ¹H NMR (500 MHz, DMSO-d₆) δ: 7.81 (s, 1H), 7.50 (d, J=7.3 Hz, 1H), 7.48 (s, 1H), 7.45 (s, 1H), 7.27 (t, J=7.7 Hz, 1H), 7.20-7.14 (m, 2H), 6.92 (d, J=8.1 Hz, 1H), 6.80-6.71 (m, 2H), 5.30 (s, 2H), 5.10 (br. s., 2H), 4.50 (s, 2H), 3.91 (br. s., 2H), 2.25 (s, 3H), 1.90 (s, 2H), 1.22 (s, 3H) Note: several resonances were obscured by residual solvent.

### Example 1017: (S)-1-(2-((3-(Aminomethyl)benzyl)oxy)-5-chloro-4-((3-(2,3-dihydrobenzo[b][1,4]dioxin-6-yl)-2-methylbenzyl)oxy)benzyl)piperidine-2-carboxylic acid, bis-trifluoroacetic acid salt

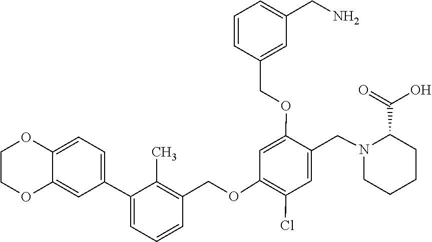 [SMILES: Cc1c(COc2cc(OCc3cccc(CN)c3)c(CN3CCCC[C@H]3C(=O)O)cc2Cl)cccc1-c1ccc2c(c1)OCCO2]

A solution of (S)-1-(2-((3-(((tert-butoxycarbonyl)amino)methyl)benzyl)oxy)-5-chloro-4-((3-(2,3-dihydrobenzo[b][1,4]dioxin-6-yl)-2-methylbenzyl)oxy)benzyl) piperidine-2-carboxylic acid (0.029 g, 0.039 mmol) in dry dichloromethane (0.5 mL) and trifluoroacetic acid (0.500 mL) was stirred for 2 hours. The reaction was split into two vials, containing approximately 0.4 mL and 0.6 mL respectively of the reaction solution. Both were dried under air stream. One vial (˜0.6 eq.) was held in reserve. The remaining vial (˜0.4 eq.) was purified via preparative LC/MS with the following conditions: Column: XBridge C18, 19×200 mm, 5-μm particles; Mobile Phase A: 5:95 methanol: water with 0.1% trifluoroacetic acid; Mobile Phase B: 95:5 methanol: water with 0.1% trifluoroacetic acid; Gradient: 20-100% B over 20 minutes, then a 5-minute hold at 100% B; Flow: 20 mL/min. Fractions containing the desired product were combined and dried via centrifugal evaporation. The yield of the product was 6.6 mg, and its estimated purity by LCMS analysis was 100% (Condition MeOH-AA, ES+) M+H=643.7, 2.73 minutes, calculated exact mass=642.25. ¹H NMR (500 MHz, DMSO-d₆) δ: 7.77 (br. s., 1H), 7.52 (s, 1H), 7.49-7.39 (m, 4H), 7.30-7.23 (m, 1H), 7.22-7.15 (m, 2H), 6.93 (d, J=8.1 Hz, 1H), 6.79-6.70 (m, 2H), 5.34-5.23 (m, 4H), 4.39 (br. s., 1H), 4.27 (s, 4H), 4.20 (d, J=13.6 Hz, 1H), 4.06 (br. s., 2H), 3.78 (br. s., 1H), 2.88 (s, 1H), 2.06 (br. s., 1H), 1.67 (br. s., 4H), 1.47 (br. s., 1H); some peaks were solvent obscured.

### Intermediate: Methyl 3-(5-(hydroxymethyl)pyridine-3-sulfonamido)propanoate

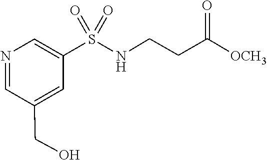 [SMILES: COC(=O)CCNS(=O)(=O)c1cncc(CO)c1]

A suspension of methyl 3-aminopropanoate hydrochloride (0.382 g, 2.74 mmol) in acetonitrile (5.0 mL) was stirred for 2 hours with solid potassium carbonate (0.488 g, 3.53 mmol), forming a thick slurry while stirring. Separately, a solution of (5-(benzylthio)pyridin-3-yl)methanol (0.204 g, 0.882 mmol), water (0.079 mL, 4.41 mmol) and acetic acid (0.252 mL, 4.41 mmol) in acetonitrile (5.0 mL) was cooled (0° C. ice/water bath), then treated with 1,3-dichloro-5,5-dimethylimidazolidine-2,4-dione (0.521 g, 2.65 mmol). The mixture was stirred for 5 minutes, warmed to room temperature, and stirred for 10 minutes. The mixture was quenched with the previously free-based methyl 3-aminopropanoate slurry. The reaction was stirred for 16 hours. The reaction was filtered through a celite pad, and the filtrate was concentrated by rotary evaporator. The residue was dissolved in minimal dichloromethane with a few drops of methanol to aid in dissolution, then purified by biotage (RediSep 24 g SiO₂, 0% (3 CV), 0-20% (15 CV), 20% (2 CV), methanol in dichloromethane). Product fractions were pooled and concentrated. The resulting waxy solid was dissolved in ethyl acetate (50 mL), washed with water (3×50 mL), then the organic layer was dried over sodium sulfate, filtered, and concentrated under reduced pressure. The combined aqueous layer was extracted with ethyl acetate (3×75 mL), and the combined organic extracts were dried over sodium sulfate, filtered, and concentrated under reduced pressure. The residue was then suspended in dichloromethane, and solids were removed by filtration. The filtrate was concentrated and the residue was used as-is in the following experiment. LCMS (Condition ACN-TFA, ES+) M+H=275.1, 0.51 minutes, calculated exact mass=274.06. ¹H NMR (400 MHz, CDCl₃) δ: 8.93 (d, J=2.0 Hz, 1H), 8.74 (d, J=1.5 Hz, 1H), 8.21 (t, J=2.0 Hz, 1H), 6.14 (t, J=6.4 Hz, 1H), 4.82 (s, 2H), 3.66 (s, 3H), 3.25 (q, J=6.2 Hz, 2H), 2.58 (t, J=6.1 Hz, 2H).

### Intermediate: Methyl 3-(5-(chloromethyl)pyridine-3-sulfonamido)propanoate

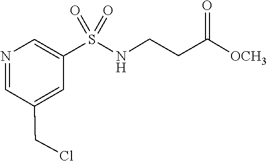 [SMILES: COC(=O)CCNS(=O)(=O)c1cncc(CCl)c1]

A solution of methyl 3-(5-(hydroxymethyl)pyridine-3-sulfonamido)propanoate (0.099 g, 0.361 mmol) in dry dichloromethane (3 mL) was treated with thionyl chloride (0.263 mL, 3.61 mmol). The solution became a suspension over several minutes. The mixture was stirred for 2 hours, then concentrated under reduced pressure affording the product as a white solid, which was used immediately in the following step. LCMS (Condition ACN-TFA, ES+) M+H=293.0, 0.69 minutes, calculated exact mass=292.03.

### Intermediate: Methyl 3-(5-((4-chloro-5-((3-(2,3-dihydrobenzo[b][1,4]dioxin-6-yl)-2-methylbenzyl)oxy)-2-formylphenoxy)methyl)pyridine-3-sulfonamido)propanoate

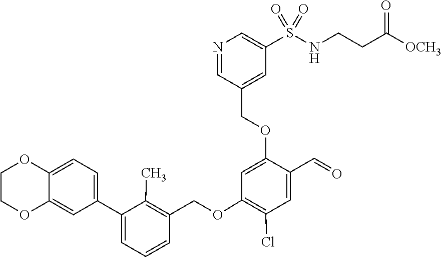 [SMILES: COC(=O)CCNS(=O)(=O)c1cncc(COc2cc(OCc3cccc(-c4ccc5c(c4)OCCO5)c3C)c(Cl)cc2C=O)c1]

A solution of 5-chloro-4-((3-(2,3-dihydrobenzo[b][1,4]dioxin-6-yl)-2-methylbenzyl)oxy)-2-hydroxybenzaldehyde (0.080 g, 0.195 mmol) and methyl 3-(5-(chloromethyl)pyridine-3-sulfonamido)propanoate (0.106 g, 0.361 mmol) in dry N,N-dimethylformamide (3 mL) was treated with cesium carbonate (0.190 g, 0.584 mmol) and sodium iodide (3 mg, 0.019 mmol), then heated (75° C. oil bath) for 2.5 hours. The reaction was allowed to cool to room temperature with stirring for 16 hours. The mixture was diluted with ethyl acetate (20 mL), then washed with water (3×20 mL), brine, and finally dried (Na₂SO₂), filtered, and concentrated under reduced pressure. The residue was purified by biotage (RediSep 12 g SiO₂, 0% (3 CV), 0-20% (15 CV), 20% (2 CV), methanol in dichloromethane). Product fractions were pooled and concentrated under reduced pressure, affording the product (0.101 g, 0.075 mmol, 38.7% yield), which was used without additional purification in the following experiment. LCMS (Condition ACN-TFA, ES+) M+H=667.1, 1.10 minutes, calculated exact mass=666.14.

### Example 1018: (S)-2-((5-Chloro-4-((3-(2,3-dihydrobenzo[b][1,4]dioxin-6-yl)-2-methylbenzyl)oxy)-2-((5-(N-(3-methoxy-3-oxopropyl)sulfamoyl)pyridin-3-yl)methoxy)benzyl)amino)-3-hydroxy-2-methylpropanoic acid

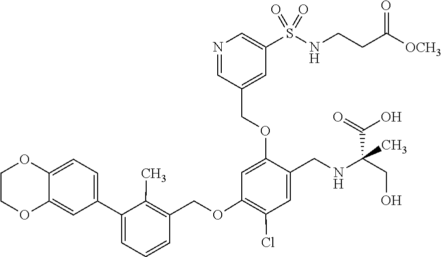 [SMILES: COC(=O)CCNS(=O)(=O)c1cncc(COc2cc(OCc3cccc(-c4ccc5c(c4)OCCO5)c3C)c(Cl)cc2CN[C@@](C)(CO)C(=O)O)c1]

A solution of methyl 3-(5-((4-chloro-5-((3-(2,3-dihydrobenzo[b][1,4]dioxin-6-yl)-2-methylbenzyl)oxy)-2-formylphenoxy)methyl)pyridine-3-sulfonamido)propanoate (0.050 g, 0.075 mmol) and (S)-2-amino-3-hydroxy-2-methylpropanoic acid (0.052 g, 0.437 mmol) in dry N,N-dimethylformamide (0.993 mL) and acetic acid (0.110 mL) was stirred for 1 hour, then treated with sodium cyanoborohydride (0.050 g, 0.796 mmol). The mixture was stirred for 16 hours. The reaction was filtered (0.45 μm syringe tip filter) and purified via preparative LCMS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-70% B over 20 minutes, then a 5-minute hold at 100% B; Flow: 20 mL/min. Fractions containing the desired product were combined and dried via centrifugal evaporation. The yield of the product was 9.4 mg, and its estimated purity by LCMS analysis was 96% (Condition MeOH-AA, ES+) M+H=770.8, 2.69 minutes, calculated exact mass=769.21. ¹H NMR (500 MHz, DMSO-d₆) δ: 8.87 (d, J=9.9 Hz, 2H), 8.77 (s, 1H), 7.57 (s, 1H), 7.50 (d, J=7.7 Hz, 1H), 7.29-7.24 (m, 1H), 7.21 (s, 1H), 7.18 (d, J=7.7 Hz, 1H), 6.92 (d, J=8.4 Hz, 1H), 6.80-6.68 (m, 2H), 5.38 (s, 2H), 5.32 (s, 2H), 4.27 (s, 4H), 4.14 (d, J=12.5 Hz, 1H), 4.02 (d, J=12.8 Hz, 1H), 3.70 (d, J=12.1 Hz, 1H), 3.58 (d, J=11.7 Hz, 1H), 3.06-2.99 (m, 2H), 2.55 (t, J=7.2 Hz, 2H), 2.25 (s, 3H), 1.30 (s, 3H); one methyl signal is solvent obscured.

Example 1019 was prepared in substantially the same manner as Example 1018, using the appropriate amine for the reductive amination.

### Intermediate: 5-(Hydroxymethyl)pyridine-3-sulfonamide

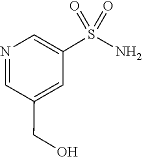 [SMILES: NS(=O)(=O)c1cncc(CO)c1]

A solution of (5-(benzylthio)pyridin-3-yl)methanol (0.512 g, 2.213 mmol), water (0.199 mL, 11.07 mmol) and acetic acid (0.634 mL, 11.07 mmol) in acetonitrile (12.0 mL) was cooled (0° C. ice/water bath), then treated with 1,3-dichloro-5,5-dimethylimidazolidine-2,4-dione (1.31 g, 6.64 mmol). The mixture was stirred for 10 minutes, then quenched with concentrated ammonium hydroxide (2.0 mL, 51.4 mmol), forming a white precipitate immediately. The reaction was stirred for 3.5 hours, then diluted with acetonitrile. Solids were removed by filtration, and the filtrate was concentrated under reduced pressure. The residue was suspended in dichloromethane with several drops of methanol added to induce dissolution and purified by biotage (RediSep 24 g SiO₂, 0% (3 CV), 0-20% (15 CV), 20% (2 CV), methanol in dichloromethane). Product fractions were pooled and concentrated under reduced pressure, affording the product (0.266 g, 1.413 mmol, 63.9% yield) as an pale yellow crystalline solid. LCMS (Condition ACN-TFA, ES+) M+H=189.1, broad peak from 0.24 to 0.34 minutes, calculated exact mass=188.03. ¹H NMR (400 MHz, CD₃OD) δ: 8.92 (d, J=2.3 Hz, 1H), 8.71 (d, J=2.0 Hz, 1H), 8.29 (t, J=2.1 Hz, 1H), 4.75 (d, J=0.5 Hz, 2H).

### Intermediate: 5-(Chloromethyl)pyridine-3-sulfonamide

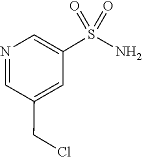 [SMILES: NS(=O)(=O)c1cncc(CCl)c1]

A suspension of 5-(hydroxymethyl)pyridine-3-sulfonamide (0.212 g, 1.126 mmol) in dry dichloromethane (6.0 mL) was treated with thionyl chloride (0.493 mL, 6.76 mmol) and the mixture was stirred for 2 hours, during which time most solids appeared to remain undissolved. Additional thionyl chloride (˜2 mL, ˜26 mmol) was added and the reaction was stirred for several days. The reaction was concentrated under reduced pressure and the residue was dried twice from dichloromethane. The dry residue was treated with neat thionyl chloride (6 mL) and stirred for 3 hours, then concentrated under reduced pressure, affording a mixture of desired chloride product and unreacted starting material. This was used immediately in the following reaction. ¹H NMR (400 MHz, DMSO-d₆) δ: 8.94 (d, J=2.3 Hz, 1H), 8.86 (m, 1H), 8.28 (t, J=2.0 Hz, 1H), 4.95 (s, 2H).

### Intermediate: 5-((4-Chloro-5-((3-(2,3-dihydrobenzo[b][1,4]dioxin-6-yl)-2-methylbenzyl)oxy)-2-formylphenoxy)methyl)pyridine-3-sulfonamide

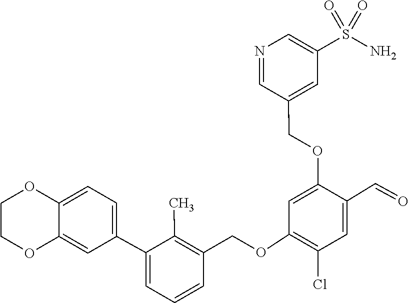 [SMILES: Cc1c(COc2cc(OCc3cncc(S(N)(=O)=O)c3)c(C=O)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-hydroxybenzaldehyde (0.130 g, 0.316 mmol), 5-(chloromethyl) pyridine-3-sulfonamide (0.140 g, 0.6756 mmol), cesium carbonate (0.361 g, 1.107 mmol) and sodium iodide (5 mg, 0.04 mmol) in dry N,N-dimethylformamide (4.0 mL) was stirred with heating (70° C. oil bath) for 90 minutes. The reaction mixture was cooled, diluted with ethyl acetate (20 mL), then washed with water (2×20 mL) and brine. The organic layer was dried over sodium sulfate, filtered, and concentrated under reduced pressure to afford a deep yellow solid. The residue was purified by biotage (RediSep 12 g SiO₂, 0% (5 CV), 0-20% (15 CV), 20% (5 CV), methanol in dichloromethane). Product fractions were pooled and concentrated and the residue was re-purified as above. Product fractions were pooled and concentrated under reduced pressure, affording the product (0.099 g, 0.170 mmol, 53.8% yield) as a bright yellow solid. LCMS (Condition ACN-TFA, ES+) M+H=581.1, 1.05 minutes, calculated exact mass=580.11.

### Example 1019: (S)-2-((5-Chloro-4-((3-(2,3-dihydrobenzo[b][1,4]dioxin-6-yl)-2-methylbenzyl)oxy)-2-((5-sulfamoylpyridin-3-yl)methoxy)benzyl)amino)-3-hydroxy-2-methylpropanoic acid

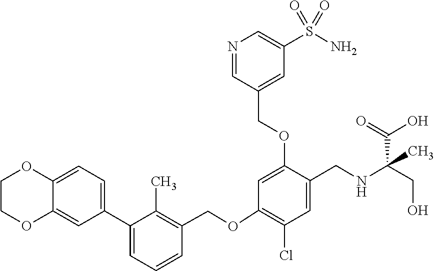 [SMILES: Cc1c(COc2cc(OCc3cncc(S(N)(=O)=O)c3)c(CN[C@@](C)(CO)C(=O)O)cc2Cl)cccc1-c1ccc2c(c1)OCCO2]

The yield of the product was 6.2 mg, and its estimated purity by LCMS analysis was 97% (Condition ACN-AA, ES+) M+H=684.8, 1.64 minutes, calculated exact mass=683.17. ¹H NMR (500 MHz, DMSO-d₆) δ: 8.88 (s, 1H), 8.86 (s, 1H), 8.78 (s, 1H), 7.57 (s, 1H), 7.50 (d, J=7.3 Hz, 1H), 7.30-7.24 (m, 1H), 7.22 (s, 1H), 7.18 (d, J=7.7 Hz, 1H), 6.92 (d, J=8.1 Hz, 1H), 6.79-6.71 (m, 2H), 5.38 (s, 2H), 5.32 (s, 2H), 4.27 (s, 4H), 4.16-4.10 (m, 1H), 4.02 (d, J=12.5 Hz, 1H), 3.69 (d, J=11.7 Hz, 1H), 3.58 (d, J=11.4 Hz, 1H), 2.25 (s, 3H), 1.29 (s, 3H).

### Example 1020: (S)-2-((2-((5-(N-(2-Carboxyethyl)sulfamoyl)pyridin-3-yl)methoxy)-5-chloro-4-((3-(2,3-dihydrobenzo[b]1[1,4]dioxin-6-yl)-2-methylbenzyl)oxy)benzyl)amino)-3-hydroxy-2-methylpropanoic acid

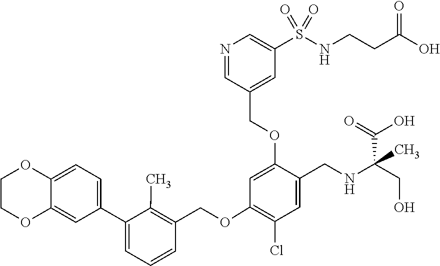 [SMILES: Cc1c(COc2cc(OCc3cncc(S(=O)(=O)NCCC(=O)O)c3)c(CN[C@@](C)(CO)C(=O)O)cc2Cl)cccc1-c1ccc2c(c1)OCCO2]

A solution of (S)-2-((5-chloro-4-((3-(2,3-dihydrobenzo[b][1,4]dioxin-6-yl)-2-methylbenzyl)oxy)-2-((5-(N-(3-methoxy-3-oxopropyl)sulfamoyl)pyridin-3-yl)methoxy) benzyl)amino)-3-hydroxy-2-methylpropanoic acid (0.0079 g, 10.26 μmol) in methanol (0.5 mL) was treated with lithium hydroxide monohydrate (4 mg, 0.1 mmol), and the mixture was heated (60° C. oil bath) for 90 minutes. The reaction was cooled, then filtered (0.45 μm syringe tip filter) and the filtrate was purified via preparative LCMS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 25-65% B over 15 minutes, then a 5-minute hold at 100% B; Flow: 20 mL/min. Fractions containing the desired product were combined and dried via centrifugal evaporation, affording the product (0.0066 g, 8.73 μmol, 85% yield). LCMS (Condition ACN-AA,ES+) M+H=755.90, 1.53 minutes, calculated exact mass=755.19. ¹H NMR (500 MHz, DMSO-d₆) δ: 8.89 (s, 1H), 8.86 (s, 1H), 8.71 (br. s., 1H), 7.55 (s, 1H), 7.50 (d, J=7.3 Hz, 1H), 7.29-7.23 (m, 1H), 7.23-7.14 (m, 2H), 6.92 (d, J=8.1 Hz, 1H), 6.81-6.71 (m, 2H), 5.39 (s, 2H), 5.31 (s, 2H), 4.27 (s, 4H), 4.11 (d, J=12.1 Hz, 1H), 4.06-3.98 (m, 1H), 3.69 (d, J=11.0 Hz, 1H), 2.96 (br. s., 2H), 2.39 (t, J=7.0 Hz, 2H), 2.25 (s, 3H), 1.29 (s, 3H).

Analytical LCMS conditions for Example 1021 to Example 1059: Condition ACN-AA: Column Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm.

Condition ACN-TFA: Column Waters Acquity UPLC BEH C18, 2.1×50 mm, 1.7-μm particles; Mobile Phase A: 5:95 acetonitrile:water with 0.10% trifluoroacetic acid; Mobile Phase B: 95:5 acetonitrile:water with 0.1% trifluoroacetic acid; Temperature: 50° C.; Gradient: 0-100% B over 3 minutes, then a 0.75-minute hold at 100% B; Flow: 1.0 mL/min; Detection: UV at 220 nm.

Estimated Purity: Two analytical LCMS injections were used to determine the final purity, and estimated purity is the average of the two results, using the preceding conditions.

### Intermediate: Ethyl 5-((4-chloro-5-((3-(2,3-dihydrobenzo[b][1,4]dioxin-6-yl)-2-methylbenzyl)oxy)-2-formylphenoxy)methyl)nicotinate

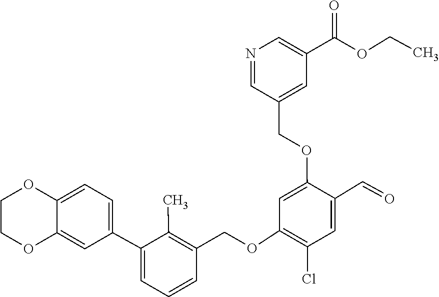 [SMILES: CCOC(=O)c1cncc(COc2cc(OCc3cccc(-c4ccc5c(c4)OCCO5)c3C)c(Cl)cc2C=O)c1]

A solution of 5-chloro-4-((3-(2,3-dihydrobenzo[b][1,4]dioxin-6-yl)-2-methylbenzyl)oxy)-2-hydroxybenzaldehyde (0.285 g, 0.694 mmol) and ethyl 5-(bromomethyl)nicotinate (0.182 g, 0.746 mmol) in DMF (6 mL) was treated with cesium carbonate (0.452 g, 1.387 mmol) and sodium iodide (10.40 mg, 0.069 mmol), stirred with heating (75° C. oil bath) for 2.5 hrs, then slowly cooled to room temperature and stirred for 16 hours. The reaction mixture was diluted with EtOAc (25 mL) and washed with water (2×25 mL), brine, then dried (Na₂SO₄), filtered, and concentrated under reduced pressure. The residue was purified by biotage (RediSep 24 g SiO₂, 0% (3 CV), 0-100% (15 CV), 100% (2 CV), EtOAc in hexanes). Product fractions were pooled and concentrated under reduced pressure, affording the product (0.275 g, 0.479 mmol, 69% yield) as a viscous oil that crystallized upon standing. LCMS (ES+) M+H=574.2. ¹H NMR (400 MHz, CDCl₃) δ: 10.31 (s, 1H), 9.25 (d, J=1.8 Hz, 1H), 8.87 (d, J=2.0 Hz, 1H), 8.41 (t, J=2.1 Hz, 1H), 7.92 (s, 1H), 7.45-7.38 (m, 1H), 7.26 (s, 1H), 6.92 (d, J=8.3 Hz, 1H), 6.84 (d, J=2.0 Hz, 1H), 6.81-6.75 (m, 1H), 6.68 (s, 1H), 5.25 (s, 2H), 5.23 (s, 2H), 4.45 (q, J=7.0 Hz, 2H), 4.32 (s, 4H), 2.30 (s, 3H), 1.43 (t, J=7.2 Hz, 3H).

### Intermediate: 5-((4-Chloro-5-((3-(2,3-dihydrobenzo[b][1,4]dioxin-6-yl)-2-methylbenzyl)oxy)-2-formylphenoxy)methyl)nicotinic acid

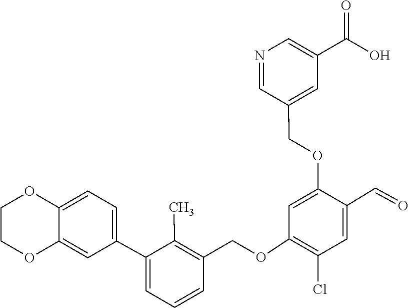 [SMILES: Cc1c(COc2cc(OCc3cncc(C(=O)O)c3)c(C=O)cc2Cl)cccc1-c1ccc2c(c1)OCCO2]

A solution of ethyl 5-((4-chloro-5-((3-(2,3-dihydrobenzo[b][1,4]dioxin-6-yl)-2-methylbenzyl)oxy)-2-formylphenoxy)methyl)nicotinate (0.275 g, 0.479 mmol) in MeOH (2.0 mL) and THF (2.0 mL) was treated with 1.0 M aq. NaOH (3.0 mL, 3.00 mmol), and the mixture was stirred for 2 hrs. The mixture was concentrated to remove organic solvents and then diluted with CH₂C₁₂ and water. The mixture was treated with drops of TFA until dissolution of solids was complete, then the layers were separated. The aq. layer was washed with additional CH₂Cl₂, then EtOAc, and the combined organic layers were diluted with MeOH to effect complete dissolution of the turbid mixture. The solution was dried (Na₂SO₄), filtered, and concentrated under reduced pressure, affording the product (0.263 g, 0.482 mmol, 101% yield) as a white powdery solid. LCMS (ES+) M+H=546.2. ¹H NMR (500 MHz, CDCl₃) δ: 10.25 (s, 1H), 9.19 (d, J=1.8 Hz, 1H), 8.80 (d, J=2.0 Hz, 1H), 8.42 (s, 1H), 7.87 (s, 1H), 7.43-7.35 (m, 1H), 7.24-7.16 (m, 2H), 6.88 (d, J=8.2 Hz, 1H), 6.79 (d, J=1.8 Hz, 1H), 6.74 (dd, J=8.2, 2.0 Hz, 1H), 6.68 (s, 1H), 5.22 (s, 2H), 5.20 (s, 2H), 4.27 (s, 4H), 2.25 (s, 3H); Note: sample prepared in CDCl₃ with one drop CD₃OD to effect complete dissolution.

### Intermediate: (S)-5-((4-Chloro-5-((3-(2,3-dihydrobenzo[b][1,4]dioxin-6-yl)-2-methylbenzyl)oxy)-2-((2-(methoxycarbonyl)piperidin-1-yl)methyl)phenoxy)methyl)nicotinic acid

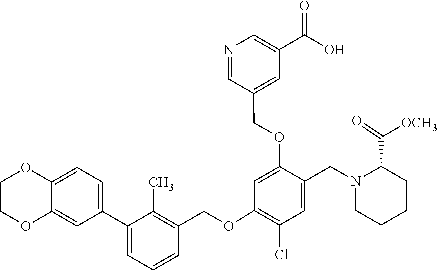 [SMILES: COC(=O)[C@@H]1CCCCN1Cc1cc(Cl)c(OCc2cccc(-c3ccc4c(c3)OCCO4)c2C)cc1OCc1cncc(C(=O)O)c1]

A solution of 5-((4-chloro-5-((3-(2,3-dihydrobenzo[b][1,4]dioxin-6-yl)-2-methylbenzyl)oxy)-2-formylphenoxy)methyl)nicotinic acid (0.143 g, 0.262 mmol) and (S)-methyl piperidine-2-carboxylate hydrochloride (0.166 g, 0.924 mmol) in dry DMF (4 mL) was treated with triethylamine (0.128 mL, 0.917 mmol) and stirred for 10 min. To the mixture was added acetic acid (0.075 ml, 1.310 mmol), and the mixture was stirred for 1 hr, then sodium cyanoborohydride (0.058 g, 0.917 mmol) was added and the reaction was stirred for 16 hours. The reaction was diluted with EtOAc (30 mL) and washed with water (30 mL) followed by brine (2×30 mL). The organic layer was dried (Na₂SO₄), filtered, and concentrated under reduced pressure, affording a viscous oil. The mixture was filtered (0.45 μm syringe tip filter). The residue was purified by biotage (24 g SiO₂, 0-20% (30 CV), methanol in dichloromethane). Product fractions were pooled and concentrated under reduced pressure, affording the product (0.055 g, 0.082 mmol, 31.2% yield) as a white lyophilized solid. LCMS (ESI+) M+H=673.05.

### Intermediate: (S)-Methyl 1-(5-chloro-4-((3-(2,3-dihydrobenzo[b][1,4]dioxin-6-yl)-2-methylbenzyl)oxy)-2-((5-((methylsulfonyl)carbamoyl)pyridin-3-yl)methoxy)benzyl)piperidine-2-carboxylate

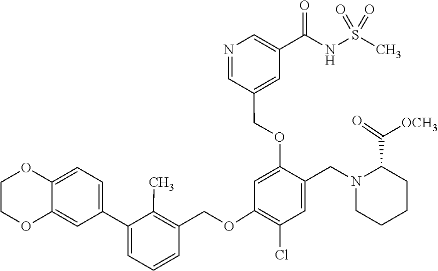 [SMILES: COC(=O)[C@@H]1CCCCN1Cc1cc(Cl)c(OCc2cccc(-c3ccc4c(c3)OCCO4)c2C)cc1OCc1cncc(C(=O)NS(C)(=O)=O)c1]

A solution of (S)-5-((4-chloro-5-((3-(2,3-dihydrobenzo[b][1,4]dioxin-6-yl)-2-methylbenzyl)oxy)-2-((2-(methoxycarbonyl)piperidin-1-yl)methyl)phenoxy)methyl) nicotinic acid (0.056 g, 0.083 mmol) and N,N′-carbonyldiimdazole (0.015 g, 0.092 mmol) in THF (0.5 mL) was heated (70° C. oil bath) for 1 hr. The reaction was cooled, then treated with a pre-mixed solution of methanesulfonamide (0.012 g, 0.125 mmol) and DBU (0.025 mL, 0.166 mmol) in THF (0.5 mL). The reaction was stirred with heating (50° C. oil bath) for 90 minutes, then cooled and concentrated under reduced pressure. The residue was purified by biotage (RediSep 12 g SiO₂, 0% (3 CV), 0-20% (20 CV), 20% (2 CV), MeOH in CH₂Cl₂). Product fractions were pooled and concentrated under reduced pressure, affording semi-purified product (0.58 g) as a clear oil. The material was dissolved in CH₂Cl₂ (10 mL) and washed with 0.02 M aqueous HCl (10 mL), then water. The organic layer was dried over sodium sulfate, filtered, and concentrated under reduced pressure, to afford the product (0.035 g, 0.047 mmol, 56.1% yield). LCMS (Condition ACN-AA, ES+) M+H=750.1, 2.13 minutes, calculated exact mass=749.22. ¹H NMR (500 MHz, DMSO-d₆) δ: 9.00 (br. s., 1H), 8.68 (br. s., 1H), 8.31 (s, 1H), 7.47 (d, J=7.3 Hz, 1H), 7.32 (s, 1H), 7.25 (t, J=7.5 Hz, 1H), 7.17 (d, J=7.7 Hz, 1H), 7.14 (s, 1H), 6.92 (d, J=8.1 Hz, 1H), 6.78 (s, 1H), 6.76 (d, J=8.4 Hz, 1H), 5.28 (s, 2H), 5.23 (s, 2H), 4.28 (s, 4H), 3.58 (s, 3H), 3.55-3.45 (m, 2H), 3.24 (br. s., 1H), 2.85 (s, 3H), 2.81 (d, J=7.3 Hz, 1H), 2.24 (s, 3H), 2.21 (br. s., 1H), 1.73 (br. s, 2H), 1.48-1.32 (m, 4H).

### Example 1021: (S)-1-(5-Chloro-4-((3-(2,3-dihydrobenzo[b][1,4]dioxin-6-yl)-2-methylbenzyl)oxy)-2-((5-((methylsulfonyl)carbamoyl)pyridin-3-yl)methoxy)benzyl)piperidine-2-carboxylic acid

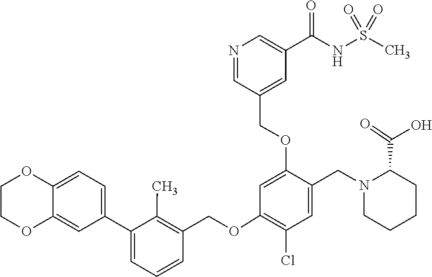 [SMILES: Cc1c(COc2cc(OCc3cncc(C(=O)NS(C)(=O)=O)c3)c(CN3CCCC[C@H]3C(=O)O)cc2Cl)cccc1-c1ccc2c(c1)OCCO2]

A solution of (S)-methyl 1-(5-chloro-4-((3-(2,3-dihydrobenzo[b][1,4]dioxin-6-yl)-2-methylbenzyl)oxy)-2-((5-((methylsulfonyl)carbamoyl)pyridin-3-yl)methoxy)benzyl) piperidine-2-carboxylate (0.035 g, 0.047 mmol) in tetrahydrofuran (0.5 mL) and methanol (0.5 mL) was treated with lithium hydroxide monohydrate (0.022 g, 0.524 mmol) and two drops of water, and heated (65° C. oil bath) for 4.5 hours. The reaction was stirred for 90 min, then cooled, filtered (0.45 μm syringe tip filter). The crude material was purified via preparative LC/MS with the following conditions: Column: XBridge C18, 19×mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-50% B over 30 minutes, then a 5-minute hold at 100% B; Flow: 20 mL/min. Fractions containing the desired product were combined and dried via centrifugal evaporation, affording the product. The estimated purity was 100% (Condition ACN-AA, ES+) M+H=736.1, 1.51 minutes, calculated exact mass=735.20. ¹H NMR (500 MHz, DMSO-d₆) δ: 9.00 (s, 1H), 8.70 (s, 1H), 8.33 (s, 1H), 7.47 (d, J=7.7 Hz, 1H), 7.45 (s, 1H), 7.28-7.22 (m, 1H), 7.20-7.15 (m, 2H), 6.92 (d, J=8.1 Hz, 1H), 6.79 (s, 1H), 6.76 (d, J=8.4 Hz, 1H), 5.31 (s, 2H), 5.25 (s, 2H), 3.79 (d, J=13.9 Hz, 1H), 3.68 (d, J=13.9 Hz, 1H), 3.17 (br. s., 1H), 2.94-2.91 (m, 1H), 2.89 (s, 4H), 2.73 (s, 3H), 2.35 (br. s., 1H), 2.25 (s, 3H), 1.80 (br. s., 1H), 1.69 (br. s., 1H), 1.49 (br. s., 3H), 1.37 (br. s., 1H).

### Intermediate: 2-(2-Bromoethoxy)-5-chloro-4-((3-(2,3-dihydrobenzo[b][1,4]dioxin-6-yl)-2-methylbenzyl)oxy)benzaldehyde

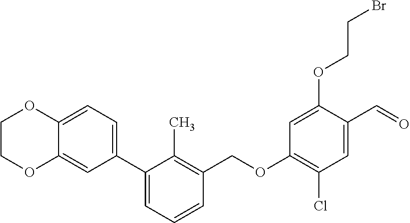 [SMILES: Cc1c(COc2cc(OCCBr)c(C=O)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-hydroxybenzaldehyde (1.146 g, 2.79 mmol) in dry N,N-dimethylformamide (30 ml) was treated with cesium carbonate (1.363 g, 4.18 mmol) followed by 1,2-dibromoethane (2.62 g, 13.95 mmol). The mixture was stirred with heating (70° C. oil bath) for 5 hours then cooled and stirred for 16 hours. The reaction was diluted with ethyl acetate (50 mL) and washed with water (2×50 mL) followed by brine (50 mL). The organic layer was dried over sodium sulfate, filtered, and concentrated under reduced pressure. The residue was purified by biotage (RediSep 40 g SiO₂, 0% (3 CV), 0-50% (15 CV), 50% (3 CV), ethyl acetate in hexanes). Product fractions were pooled and concentrated under reduced pressure, affording the product (0.980 g, 1.893 mmol, 67.9% yield) as a white solid. LCMS (ES+) M+H=519.0. ¹H NMR (400 MHz, CDCl₃) δ: 10.34 (s, 1H), 7.90 (s, 1H), 7.44 (dd, J=6.0, 3.0 Hz, 1H), 7.28 (m, 1H), 7.26 (m, 1H), 6.92 (d, J=8.3 Hz, 1H), 6.83 (d, J=2.0 Hz, 1H), 6.78 (dd, J=8.3, 2.0 Hz, 1H), 6.58 (s, 1H), 5.24 (s, 2H), 4.41 (t, J=6.0 Hz, 2H), 4.32 (s, 4H), 3.70 (t, J=6.0 Hz, 2H), 2.30 (s, 3H).

### Intermediate: (R)-5-Chloro-4-((3-(2,3-dihydrobenzo[b][1,4]dioxin-6-yl)-2-methylbenzyl)oxy)-2-(2-(3-hydroxypyrrolidin-1-yl)ethoxy)benzaldehyde

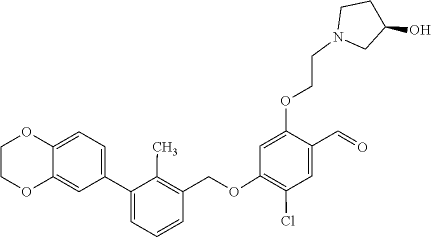 [SMILES: Cc1c(COc2cc(OCCN3CC[C@@H](O)C3)c(C=O)cc2Cl)cccc1-c1ccc2c(c1)OCCO2]

A solution of 2-(2-bromoethoxy)-5-chloro-4-((3-(2,3-dihydrobenzo[b][1,4]dioxin-6-yl)-2-methylbenzyl)oxy)benzaldehyde (0.166 g, 0.321 mmol) in dry N,N-dimethylformamide (3.0 mL) was treated with (R)-pyrrolidin-3-ol hydrochloride (0.119 g, 0.962 mmol), potassium carbonate (0.266 g, 1.924 mmol) and sodium iodide (4.81 mg, 0.032 mmol). The mixture was stirred with heating (65° C. oil bath) for 10 hours. The reaction was cooled, diluted with ethyl acetate (20 mL) and washed with water (20 mL) followed by brine (20 mL). The organic layer was dried over sodium sulfate, filtered, and concentrated under reduced pressure. The residue was triturated with diethyl ether to afford the product (0.084 g, 0.160 mmol, 50.0% yield) as an amber glassy solid. LCMS (ES+) M+H=524.1. ¹H NMR (400 MHz, CDCl₃) δ: 10.30 (s, 1H), 7.88 (s, 1H), 7.45 (dd, J=5.9, 2.9 Hz, 1H), 7.26 (s, 1H), 6.92 (d, J=8.3 Hz, 1H), 6.83 (d, J=2.0 Hz, 1H), 6.79 (dd, J=8.2, 2.1 Hz, 1H), 6.61 (s, 1H), 5.23 (s, 2H), 4.43-4.36 (m, 1H), 4.32 (s, 5H), 4.22 (t, J=5.6 Hz, 2H), 3.08-2.94 (m, 3H), 2.82 (d, J=10.3 Hz, 1H), 2.67 (dd, J=10.0, 5.0 Hz, 1H), 2.49-2.40 (m, 1H), 2.30 (s, 3H), 2.27-2.16 (m, 1H), 1.83-1.73 (m, 1H).

### Example 1022: (R)-2-((5-Chloro-4-((3-(2,3-dihydrobenzo[b][1,4]dioxin-6-yl)-2-methylbenzyl)oxy)-2-(2-((R)-3-hydroxypyrrolidin-1-yl)ethoxy)benzyl)amino)-3-hydroxy-2-methylpropanoic acid

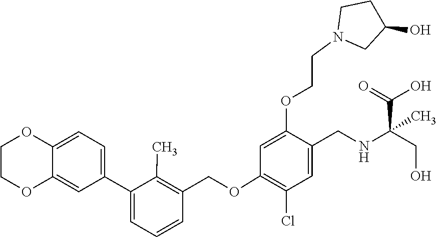 [SMILES: Cc1c(COc2cc(OCCN3CC[C@@H](O)C3)c(CN[C@](C)(CO)C(=O)O)cc2Cl)cccc1-c1ccc2c(c1)OCCO2]

A solution of (R)-5-chloro-4-((3-(2,3-dihydrobenzo[b][1,4]dioxin-6-yl)-2-methylbenzyl)oxy)-2-(2-(3-hydroxypyrrolidin-1-yl)ethoxy)benzaldehyde (0.040 g, 0.076 mmol) and (R)-2-amino-3-hydroxy-2-methylpropanoic acid (0.018 g, 0.153 mmol) in dry N,N-dimethylformamide (1.0 mL) was treated with acetic acid (0.022 mL, 0.382 mmol), stirred for 30 minutes, then treated with sodium cyanoborohydride (9.59 mg, 0.153 mmol). The mixture was stirred for 16 hours. The reaction was filtered (0.45 μm syringe-tip filter) and purified via preparative LCMS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 15-80% B over 25 minutes, then a 5-minute hold at 100% B; Flow: 20 mL/min. Fractions containing the desired product were combined and dried via centrifugal evaporation, affording the product (0.0275 g, 0.044 mmol, 57.4% yield). The estimated purity by LCMS analysis was 100%. LCMS (Condition ACN-AA, ES+) M+H=627.1, 1.55 minutes, calculated exact mass=626.24. ¹H NMR (500 MHz, DMSO-d₆) δ: 7.48 (d, J=7.3 Hz, 1H), 7.45 (s, 1H), 7.29-7.23 (m, 1H), 7.18 (d, J=6.6 Hz, 1H), 7.02 (s, 1H), 6.92 (d, J=8.1 Hz, 1H), 6.79-6.71 (m, 2H), 5.28 (s, 2H), 4.20 (t, J=5.3 Hz, 1H), 3.93-3.83 (m, 2H), 3.63 (d, J=11.0 Hz, 1H), 3.52 (d, J=11.4 Hz, 1H), 3.00-2.85 (m, 3H), 2.84-2.76 (m, 1H), 2.69-2.60 (m, 1H), 2.24 (s, 3H), 2.07-1.95 (m, 1H), 1.93-1.86 (m, 7H), 1.61 (d, J=3.7 Hz, 1H), 1.25 (s, 3H).

### Example 1023: (S)-1-(5-Chloro-4-((3-(2,3-dihydrobenzo[b][1,4]dioxin-6-yl)-2-methylbenzyl)oxy)-2-(2-((R)-3-hydroxypyrrolidin-1-yl)ethoxy)benzyl)piperidine-2-carboxylic acid

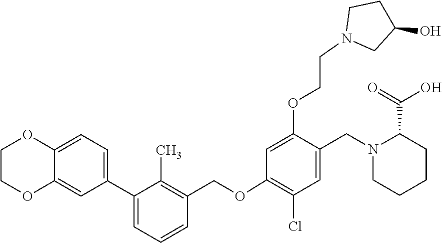 [SMILES: Cc1c(COc2cc(OCCN3CC[C@@H](O)C3)c(CN3CCCC[C@H]3C(=O)O)cc2Cl)cccc1-c1ccc2c(c1)OCCO2]

Prepared in substantially the same manner as Example 1022 above, except using (S)-piperidine-2-carboxylic acid (0.020 g, 0.153 mmol) as the amine, to afford the product (0.0238 g, 0.037 mmol, 48.9% yield). The estimated purity by LCMS analysis was 100%. LCMS (Condition ACN-AA, ES+) M+H=637.1, 1.63 minutes, calculated exact mass=636.26. ¹H NMR (500 MHz, DMSO-d₆) δ: 7.49 (d, J=7.0 Hz, 1H), 7.38 (s, 1H), 7.29-7.23 (m, 1H), 7.18 (d, J=7.7 Hz, 1H), 6.99 (s, 1H), 6.92 (d, J=8.1 Hz, 1H), 6.77 (d, J=1.8 Hz, 1H), 6.76-6.72 (m, 1H), 5.26 (s, 2H), 4.30-4.22 (m, 5H), 4.18 (t, J=5.3 Hz, 2H), 3.83 (d, J=12.8 Hz, 1H), 3.05-2.93 (m, 3H), 2.62 (d, J=11.0 Hz, 1H), 2.39-2.30 (m, 1H), 2.24 (s, 3H), 2.07-1.97 (m, 1H), 1.92-1.88 (m, 5H), 1.86-1.76 (m, 1H), 1.65 (d, J=8.8 Hz, 2H), 1.57-1.46 (m, 3H), 1.40-1.28 (m, 1H).

### Example 1024: (R)-2-((5-Chloro-4-((3-(2,3-dihydrobenzo[b][1,4]dioxin-6-yl)-2-methylbenzyl)oxy)-2-(2-(3-hydroxypyrrolidin-1-yl)ethoxy)benzyl)amino)-2-methylpropane-1,3-diol

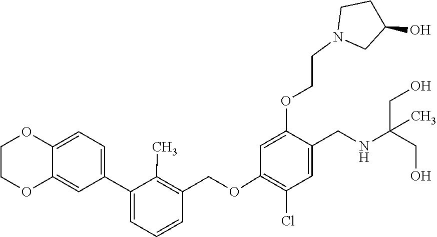 [SMILES: Cc1c(COc2cc(OCCN3CC[C@@H](O)C3)c(CNC(C)(CO)CO)cc2Cl)cccc1-c1ccc2c(c1)OCCO2]

A solution of (R)-5-chloro-4-((3-(2,3-dihydrobenzo[b][1,4]dioxin-6-yl)-2-methylbenzyl)oxy)-2-(2-(3-hydroxypyrrolidin-1-yl)ethoxy)benzaldehyde (0.040 g, 0.076 mmol) and 2-amino-2-methylpropane-1,3-diol (0.016 g, 0.153 mmol) in dry N,N-dimethylformamide (1.0 mL) was treated with acetic acid (0.022 mL, 0.382 mmol), stirred for 30 minutes, then treated with sodium cyanoborohydride (9.59 mg, 0.153 mmol). The mixture was stirred for 16 hours. The reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation, affording the product (0.0312 g, 0.049 mmol, 64.7% yield). The estimated purity by LCMS analysis was 97%. LCMS (Condition ACN-AA, ES+) M+H=613.1, 1.64 minutes, calculated exact mass=612.26.

### Intermediate: 2-(4-Bromobutoxy)-5-chloro-4-((3-(2,3-dihydrobenzo[b][1,4]dioxin-6-yl)-2-methylbenzyl)oxy)benzaldehyde

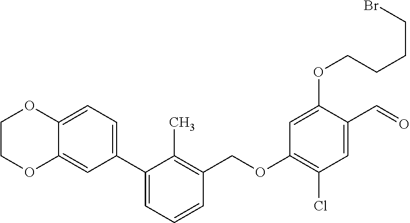 [SMILES: Cc1c(COc2cc(OCCCCBr)c(C=O)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-hydroxybenzaldehyde (0.220 g, 0.535 mmol) in dry N,N-dimethylformamide (5.0 mL) was treated with cesium carbonate (0.262 g, 0.803 mmol) followed by 1,4-dibromobutane (0.320 mL, 2.68 mmol). The mixture was stirred with heating (70° C. oil bath) for 16 hours. The reaction was diluted with ethyl acetate (50 mL) and washed with water (2×50 mL) followed by brine (20 mL). The organic layer was dried over sodium sulfate, filtered, and concentrated under reduced pressure. The residue was purified by biotage (RediSep 12 g SiO₂, 0% (3 CV), 0-50% (20 CV), 50% (2 CV), ethyl acetate in hexanes). Product fractions were pooled and concentrated under reduced pressure, affording the product (0.256 g, 0.469 mmol, 88% yield) as a white solid. LCMS (ES+) M+H=546.9. ¹H NMR (400 MHz, CDCl₃) δ: 10.30 (s, 1H), 7.88 (s, 1H), 7.45 (dd, J=5.9, 3.1 Hz, 1H), 7.28-7.27 (m, 1H), 7.26 (s, 1H), 6.93 (d, J=8.3 Hz, 1H), 6.83 (d, J=2.0 Hz, 1H), 6.78 (dd, J=8.2, 2.1 Hz, 1H), 6.59 (s, 1H), 5.24 (s, 2H), 4.32 (s, 4H), 4.14-4.09 (m, 2H), 3.48-3.41 (m, 1H), 2.30 (s, 3H), 2.09 (td, J=6.1, 3.1 Hz, 2H), 1.96 (dd, J=14.7, 6.7 Hz, 1H), 1.86-1.73 (m, 1H).

### Intermediate: (R)-5-Chloro-4-((3-(2,3-dihydrobenzo[b][1,4]dioxin-6-yl)-2-methylbenzyl)oxy)-2-(4-(3-hydroxypyrrolidin-1-yl)butoxy)benzaldehyde

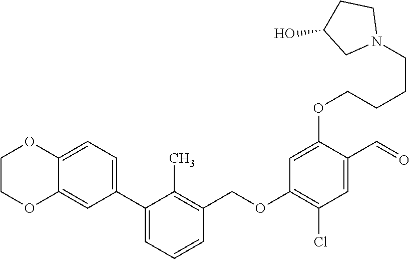 [SMILES: Cc1c(COc2cc(OCCCCN3CC[C@@H](O)C3)c(C=O)cc2Cl)cccc1-c1ccc2c(c1)OCCO2]

A solution of 2-(4-bromobutoxy)-5-chloro-4-((3-(2,3-dihydrobenzo[b][1,4]dioxin-6-yl)-2-methylbenzyl)oxy)benzaldehyde (0.220 g, 0.403 mmol) and (R)-pyrrolidin-3-ol hydrochloride (0.140 g, 1.133 mmol) in dry N,N-dimethylformamide (4.03 ml) was treated with potassium carbonate (0.215 g, 1.556 mmol) and sodium iodide (6.04 mg, 0.040 mmol). The mixture was heated (65° C. oil bath) for 3 hours, then cooled, diluted with ethyl acetate (30 mL) and washed with water (30 mL) followed by brine (30 mL). The organic layer was dried over sodium sulfate, filtered, and concentrated under reduced pressure. The residue was taken up in minimal methanol with a few drops of acetonitrile and loaded onto an SCX column. The column was eluted with several volumes of methanol, then ammonia in methanol. The ammonia fraction was concentrated under reduced pressure. The residue was purified by biotage (RediSep 24 g SiO₂, 0% (3 CV), 0-25% (30 CV), 25% (2 CV), methanol in dichloromethane). Product fractions were pooled and concentrated under reduced pressure, affording the product (0.138 g, 0.250 mmol, 62.0% yield). LCMS (199-07, ES+) M+H=552.2, 0.89 minutes, calculated exact mass=551.21. ¹H NMR (400 MHz, CDCl₃) δ: 10.30 (s, 0.3H), 7.91-7.50 (m, 1H), 7.49-7.42 (m, 1H), 7.27-7.21 (m, 2H), 6.95-6.89 (m, 1H), 6.83 (d, J=2.0 Hz, 1H), 6.78 (dd, J=8.3, 2.0 Hz, 1H), 6.61-6.53 (m, 1H), 5.57 (s, 1H), 5.25-5.10 (m, 2H), 4.39-4.32 (m, 1H), 4.32-4.30 (m, 4H), 4.13-4.07 (m, 1H), 3.98 (t, J=6.3 Hz, 1H), 2.96-2.84 (m, 1H), 2.78-2.67 (m, 1H), 2.58-2.45 (m, 3H), 2.29-2.12 (m, 3H), 1.98-1.81 (m, 4H), 1.80-1.62 (m, 6H).

### Example 1025: (S)-1-(5-Chloro-4-((3-(2,3-dihydrobenzo[b][1,4]dioxin-6-yl)-2-methylbenzyl)oxy)-2-(4-((R)-3-hydroxypyrrolidin-1-yl)butoxy)benzyl)piperidine-2-carboxylic acid

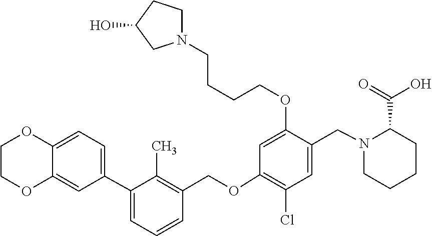 [SMILES: Cc1c(COc2cc(OCCCCN3CC[C@@H](O)C3)c(CN3CCCC[C@H]3C(=O)O)cc2Cl)cccc1-c1ccc2c(c1)OCCO2]

A solution of (R)-5-chloro-4-((3-(2,3-dihydrobenzo[b][1,4]dioxin-6-yl)-2-methylbenzyl)oxy)-2-(4-(3-hydroxypyrrolidin-1-yl)butoxy)benzaldehyde (0.044 g, 0.080 mmol) and (S)-piperidine-2-carboxylic acid (0.021 g, 0.159 mmol) in dry N,N-dimethylformamide (1.2 ml) was treated with acetic acid (0.023 ml, 0.399 mmol), stirred for 30 minutes, then treated with sodium cyanoborohydride (10.02 mg, 0.159 mmol). The reaction was stirred for 16 hours. The reaction was treated with several drops of water, then filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 25-65% B over 15 minutes, then a 5-minute hold at 100% B; Flow: 20 mL/min. Fractions containing the desired product were combined and dried via centrifugal evaporation, affording the product (0.011 g, 0.016 mmol, 20.54% yield). The estimated purity by LCMS analysis was 99%. LCMS (Condition ACN-AA, ES+) M+H=665.2, 1.68 minutes, calculated exact mass=664.29.

### Example 1026: (R)-2-((5-Chloro-4-((3-(2,3-dihydrobenzo[b][1,4]dioxin-6-yl)-2-methylbenzyl)oxy)-2-(4-((R)-3-hydroxypyrrolidin-1-yl)butoxy)benzyl)amino)-3-hydroxy-2-methylpropanoic acid

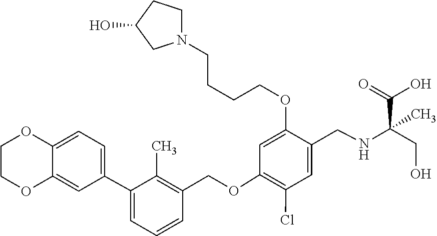 [SMILES: Cc1c(COc2cc(OCCCCN3CC[C@@H](O)C3)c(CN[C@](C)(CO)C(=O)O)cc2Cl)cccc1-c1ccc2c(c1)OCCO2]

A solution of (R)-5-chloro-4-((3-(2,3-dihydrobenzo[b][1,4]dioxin-6-yl)-2-methylbenzyl)oxy)-2-(4-(3-hydroxypyrrolidin-1-yl)butoxy)benzaldehyde (0.038 g, 0.069 mmol) and (R)-2-amino-3-hydroxy-2-methylpropanoic acid (0.016 g, 0.138 mmol) in dry N,N-dimethylformamide (1.0 ml) was treated with acetic acid (0.020 ml, 0.344 mmol), stirred for 30 minutes, then treated with sodium cyanoborohydride (8.65 mg, 0.138 mmol). The reaction was stirred for 16 hours. The reaction was treated with several drops of water, then filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 25-65% B over 15 minutes, then a 5-minute hold at 100% B; Flow: 20 mL/min. Fractions containing the desired product were combined and dried via centrifugal evaporation, affording the product (0.0102 g, 0.016 mmol, 22.6% yield). The estimated purity by LCMS analysis was 99%. LCMS (Condition ACN-AA, ES+) M+H=655.2, 1.58 minutes, calculated exact mass=654.27.

### Example 1027: (R)-2-((5-Chloro-4-((3-(2,3-dihydrobenzo[b][1,4]dioxin-6-yl)-2-methylbenzyl)oxy)-2-(4-(3-hydroxypyrrolidin-1-yl)butoxy)benzyl)amino)-2-methylpropane-1,3-diol

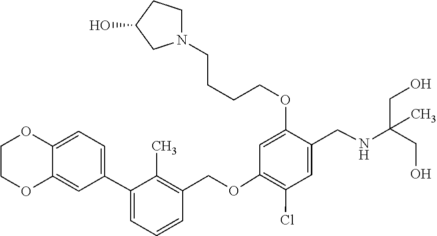 [SMILES: Cc1c(COc2cc(OCCCCN3CC[C@@H](O)C3)c(CNC(C)(CO)CO)cc2Cl)cccc1-c1ccc2c(c1)OCCO2]

A solution of (R)-5-chloro-4-((3-(2,3-dihydrobenzo[b][1,4]dioxin-6-yl)-2-methylbenzyl)oxy)-2-(4-(3-hydroxypyrrolidin-1-yl)butoxy)benzaldehyde (0.038 g, 0.069 mmol) and 2-amino-2-methylpropane-1,3-diol (0.014 g, 0.138 mmol) in dry N,N-dimethylformamide (1.0 ml) was treated with acetic acid (0.020 ml, 0.344 mmol), stirred for 30 minutes, then treated with sodium cyanoborohydride (8.65 mg, 0.138 mmol). The reaction was stirred for 16 hours. The reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation, affording (0.0162 g, 0.024 mmol, 34.9% yield). The estimated purity by LCMS analysis was 95%. LCMS (Condition ACN-AA, ES+) M+H=641.3, 1.54 minutes, calculated exact mass=640.29. ¹H NMR (500 MHz, DMSO-d₆) δ: 7.47 (d, J=8.1 Hz, 1H), 7.37 (s, 1H), 7.25 (t, J=7.7 Hz, 1H), 7.17 (d, J=7.0 Hz, 1H), 6.96-6.87 (m, 2H), 6.79-6.71 (m, 2H), 5.24 (s, 2H), 4.27 (s, 4H), 4.19 (br. s., 1H), 4.05 (t, J=6.2 Hz, 2H), 3.66 (s, 1H), 3.33 (s, 1H), 2.77-2.69 (m, 1H), 2.66-2.57 (m, 1H), 2.47 (d, J=5.5 Hz, 2H), 2.36 (d, J=6.2 Hz, 1H), 2.24 (s, 3H), 2.01-1.93 (m, 1H), 1.90 (s, 4H), 1.80-1.69 (m, 2H), 1.65-1.48 (m, 3H), 0.98 (s, 3H); several peaks were solvent obscured.

### Example 1028: (S)-1-(5-Chloro-4-((3-(2,3-dihydrobenzo[b][1,4]dioxin-6-yl)-2-methylbenzyl)oxy)-2-(4,4,4-trifluorobutoxy)benzyl)piperidine-2-carboxylic acid

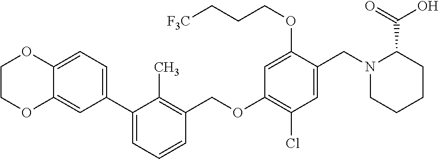 [SMILES: Cc1c(COc2cc(OCCCC(F)(F)F)c(CN3CCCC[C@H]3C(=O)O)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-(4,4,4-trifluorobutoxy)benzaldehyde (0.045 g, 0.086 mmol) and (S)-piperidine-2-carboxylic acid (0.022 g, 0.173 mmol) in dry N,N-dimethylformamide (1.270 ml) was treated with acetic acid (0.025 ml, 0.432 mmol), stirred for 30 minutes, then treated with sodium cyanoborohydride (10.86 mg, 0.173 mmol). The reaction was stirred for 16 hours. The reaction was treated with several drops of water, then filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 45-85% B over 15 minutes, then a 5-minute hold at 100% B; Flow: 20 mL/min. Fractions containing the desired product were combined and dried via centrifugal evaporation, affording the product (0.0271 g, 0.042 mmol, 49.0% yield). The estimated purity by LCMS analysis was 99%. LCMS (Condition ACN-AA, ES+) M+H=634.2, 2.00 minutes, calculated exact mass=633.21.

### Example 1029: (R)-2-((5-Chloro-4-((3-(2,3-dihydrobenzo[b][1,4]dioxin-6-yl)-2-methylbenzyl)oxy)-2-(4,4,4-trifluorobutoxy)benzyl)amino)-3-hydroxy-2-methylpropanoic acid

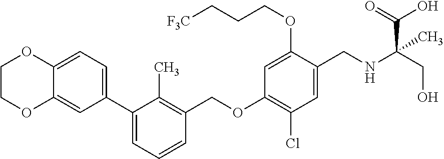 [SMILES: Cc1c(COc2cc(OCCCC(F)(F)F)c(CN[C@](C)(CO)C(=O)O)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-(4,4,4-trifluorobutoxy)benzaldehyde (0.050 g, 0.096 mmol) and (R)-2-amino-3-hydroxy-2-methylpropanoic acid (0.023 g, 0.192 mmol) in dry N,N-dimethylformamide (1.412 ml) was treated with acetic acid (0.027 ml, 0.480 mmol), stirred for 30 minutes, then treated with sodium cyanoborohydride (0.012 g, 0.192 mmol). The reaction was stirred for 16 hours. The reaction was treated with several drops of water, then filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 40-80% B over 15 minutes, then a 5-minute hold at 100% B; Flow: 20 mL/min. Fractions containing the desired product were combined and dried via centrifugal evaporation, affording the product (0.0021 g, 3.37 μmol, 3.51% yield). The estimated purity by LCMS analysis was 100%. LCMS (Condition ACN-AA, ES+) M+H=624.1, 2.19 minutes, calculated exact mass=623.19. ¹H NMR (500 MHz, DMSO-d₆) δ: 7.51-7.46 (m, 2H), 7.29-7.22 (m, 1H), 7.17 (d, J=7.7 Hz, 1H), 6.99 (s, 1H), 6.92 (d, J=8.1 Hz, 1H), 6.76 (d, J=1.8 Hz, 1H), 6.74 (dd, J=8.1, 2.2 Hz, 1H), 5.28 (s, 2H), 4.28 (s, 4H), 4.16 (t, J=5.9 Hz, 2H), 3.91 (s, 2H), 3.70-3.47 (m, 2H), 2.24 (s, 3H), 2.05-1.96 (m, 2H), 1.26 (s, 3H); some signals were solvent obscured.

### Example 1030: 2-((5-Chloro-4-((3-(2,3-dihydrobenzo[b][1,4]dioxin-6-yl)-2-methylbenzyl)oxy)-2-(4,4,4-trifluorobutoxy)benzyl)amino)-2-methylpropane-1,3-diol

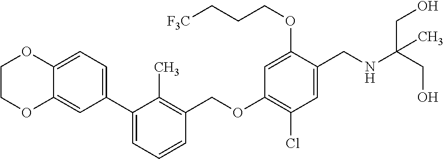 [SMILES: Cc1c(COc2cc(OCCCC(F)(F)F)c(CNC(C)(CO)CO)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-(4,4,4-trifluorobutoxy)benzaldehyde (0.050 g, 0.096 mmol) and 2-amino-2-methylpropane-1,3-diol (0.020 g, 0.192 mmol) in dry N,N-dimethylformamide (1.412 ml) was treated with acetic acid (0.027 ml, 0.480 mmol), stirred for 30 minutes, then treated with sodium cyanoborohydride (0.012 g, 0.192 mmol). The reaction was stirred for 16 hours. The reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 50-60% B over 15 minutes, then a 5-minute hold at 100% B; Flow: 20 mL/min. Fractions containing the desired product were combined and dried via centrifugal evaporation, affording the product (0.0517 g, 0.083 mmol, 87% yield). The estimated purity by LCMS analysis was 98%. LCMS (Condition ACN-AA, ES+) M+H=610.2, 2.04 minutes, calculated exact mass=609.21. ¹H NMR (500 MHz, DMSO-d₆) δ: 7.47 (d, J=7.0 Hz, 1H), 7.35 (s, 1H), 7.25 (t, J=7.5 Hz, 1H), 7.17 (d, J=7.0 Hz, 1H), 6.95-6.89 (m, 2H), 6.76 (d, J=1.8 Hz, 1H), 6.75-6.71 (m, 1H), 5.23 (s, 2H), 4.28 (s, 4H), 4.11 (t, J=5.9 Hz, 2H), 3.58 (s, 2H), 3.28 (s, 2H), 2.48-2.42 (m, 2H), 2.23 (s, 3H), 2.01-1.92 (m, 2H), 1.90 (s, 2H), 0.93 (s, 3H); some peaks were solvent obscured.

### Intermediate: (R)-5-Chloro-4-((3-(2,3-dihydrobenzo[b][1,4]dioxin-6-yl)-2-methylbenzyl)oxy)-2-(3-hydroxy-2-methylpropoxy)benzaldehyde

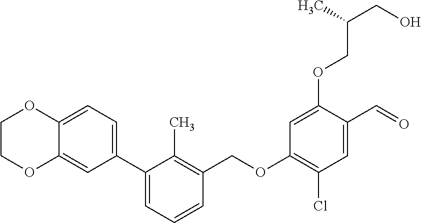 [SMILES: Cc1c(COc2cc(OC[C@H](C)CO)c(C=O)cc2Cl)cccc1-c1ccc2c(c1)OCCO2]

A suspension of 5-chloro-4-((3-(2,3-dihydrobenzo[b][1,4]dioxin-6-yl)-2-methylbenzyl)oxy)-2-hydroxybenzaldehyde (0.202 g, 0.492 mmol) in dry N,N-dimethylformamide (5.0 mL) was treated with sodium hydride, 60% oil dispersion (0.024 g, 0.590 mmol) and the mixture was stirred for 10 minutes at room temperature, until the suspension became a solution. The reaction was treated with (R)-3-bromo-2-methylpropan-1-ol (0.301 g, 1.967 mmol) and stirred with heating (60° C. oil bath) for 16 hours. The reaction mixture was diluted with saturated aqueous ammonium chloride and water, and then extracted with ethyl acetate. The organic layer was washed with water, then brine, dried over anhydrous sodium sulphate, filtered, and concentrated under reduced pressure to an amber semi-solid. The residue was purified by biotage (RediSep 24 g SiO₂, 0% (3 CV), 0-25% (25 CV), 20% (2 CV), methanol in dichloromethane). Product fractions were pooled and concentrated under reduced pressure, affording the product (0.203 g, 0.420 mmol, 85% yield). LCMS (ES+) M+Na=505.1. The residue was used directly in following reactions.

### Example 1031: (S)-1-(5-Chloro-4-((3-(2,3-dihydrobenzo[b][1,4]dioxin-6-yl)-2-methylbenzyl)oxy)-2-((R)-3-hydroxy-2-methylpropoxy)benzyl)piperidine-2-carboxylic acid

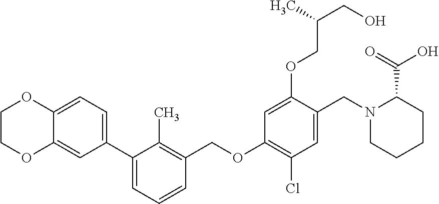 [SMILES: Cc1c(COc2cc(OC[C@H](C)CO)c(CN3CCCC[C@H]3C(=O)O)cc2Cl)cccc1-c1ccc2c(c1)OCCO2]

A suspension of (R)-5-chloro-4-((3-(2,3-dihydrobenzo[b][1,4]dioxin-6-yl)-2-methylbenzyl)oxy)-2-(3-hydroxy-2-methylpropoxy)benzaldehyde (0.087 g, 0.180 mmol) and (S)-piperidine-2-carboxylic acid (0.047 g, 0.360 mmol) in dry N,N-dimethylformamide was treated with acetic acid (0.052 ml, 0.901 mmol), stirred for 30 minutes, then treated with sodium cyanoborohydride (0.023 g, 0.360 mmol). The reaction was stirred for 3 days. The reaction was diluted with minimal drops of water to bring about dissolution, then the reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 40-90% B over 20 minutes, then a 5-minute hold at 100% B; Flow: 20 mL/min. Fractions containing the desired product were combined and dried via centrifugal evaporation, affording the product (0.0245 g, 0.041 mmol, 22.82% yield). The estimated purity by LCMS analysis was 100%. LCMS (Condition ACN-AA, ES+) M+H=596.1, 1.76 minutes, calculated exact mass=595.23. ¹H NMR (500 MHz, DMSO-d₆) δ: 7.48 (d, J=7.0 Hz, 1H), 7.41 (s, 1H), 7.26 (t, J=7.5 Hz, 1H), 7.18 (d, J=7.0 Hz, 1H), 6.95 (s, 1H), 6.92 (d, J=8.1 Hz, 1H), 6.77 (d, J=2.2 Hz, 1H), 6.75 (dd, J=8.1, 2.2 Hz, 1H), 5.25 (s, 2H), 4.28 (s, 4H), 4.01 (dd, J=9.4, 5.7 Hz, 1H), 3.92 (dd, J=9.2, 5.9 Hz, 1H), 3.78 (d, J=13.9 Hz, 1H), 3.69-3.62 (m, 1H), 3.16 (dd, J=7.7, 4.0 Hz, 1H), 2.93 (br. s., 1H), 2.36 (br. s., 1H), 2.24 (s, 3H), 2.02 (dd, J=12.5, 6.2 Hz, 1H), 1.82 (br. s., 1H), 1.77-1.66 (m, 1H), 1.51 (br. s., 3H), 1.39 (br. s., 1H), 0.98 (d, J=6.6 Hz, 3H); signal partially solvent obscured.

### Example 1032: (R)-2-((5-Chloro-4-((3-(2,3-dihydrobenzo[b][1,4]dioxin-6-yl)-2-methylbenzyl)oxy)-2-((R)-3-hydroxy-2-methylpropoxy)benzyl)amino)-3-hydroxy-2-methylpropanoic acid

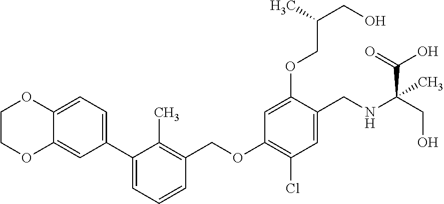 [SMILES: Cc1c(COc2cc(OC[C@H](C)CO)c(CN[C@](C)(CO)C(=O)O)cc2Cl)cccc1-c1ccc2c(c1)OCCO2]

A suspension of(R)-5-chloro-4-((3-(2,3-dihydrobenzo[b][1,4]dioxin-6-yl)-2-methylbenzyl)oxy)-2-(3-hydroxy-2-methylpropoxy)benzaldehyde (0.067 g, 0.139 mmol) and (R)-2-amino-3-hydroxy-2-methylpropanoic acid (0.033 g, 0.277 mmol) in dry N,N-dimethylformamide was treated with acetic acid (0.040 ml, 0.694 mmol), stirred for 30 minutes, then treated with sodium cyanoborohydride (0.017 g, 0.277 mmol). The mixture was stirred for 3 days. The reaction was diluted with minimal drops of water to bring about dissolution, then the reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 35-75% B over 18 minutes, then a 3-minute hold at 100% B; Flow: 20 mL/min. Fractions containing the desired product were combined and dried via centrifugal evaporation, affording the product (0.0279 g, 0.048 mmol, 34.3% yield). The estimated purity by LCMS analysis was 100%. LCMS (Condition ACN-AA, ES+) M+H=586.1, 1.68 minutes, calculated exact mass=585.21. ¹H NMR (500 MHz, DMSO-d₆) δ: 7.51-7.46 (m, 2H), 7.26 (t, J=7.5 Hz, 1H), 7.17 (d, J=7.3 Hz, 1H), 7.00 (s, 1H), 6.92 (d, J=8.1 Hz, 1H), 6.76 (d, J=2.2 Hz, 1H), 6.74 (dd, J=8.3, 2.0 Hz, 1H), 5.28 (s, 2H), 4.28 (s, 4H), 4.05 (dd, J=9.4, 6.1 Hz, 1H), 3.98-3.88 (m, 3H), 3.64 (d, J=11.0 Hz, 1H), 3.54 (d, J=11.4 Hz, 1H), 3.46 (t, J=5.1 Hz, 1H), 2.24 (s, 3H), 2.12-2.02 (m, 1H), 1.27 (s, 3H), 0.99 (d, J=6.6 Hz, 3H); signal partially solvent obscured.

### Example 1033: (R)-2-((5-Chloro-4-((3-(2,3-dihydrobenzo[b][1,4]dioxin-6-yl)-2-methylbenzyl)oxy)-2-(3-hydroxy-2-methylpropoxy)benzyl)amino)-2-methylpropane-1,3-diol

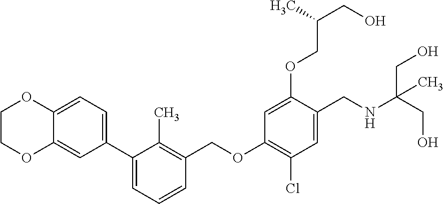 [SMILES: Cc1c(COc2cc(OC[C@H](C)CO)c(CNC(C)(CO)CO)cc2Cl)cccc1-c1ccc2c(c1)OCCO2]

A solution of (R)-5-chloro-4-((3-(2,3-dihydrobenzo[b][1,4]dioxin-6-yl)-2-methylbenzyl)oxy)-2-(3-hydroxy-2-methylpropoxy)benzaldehyde (0.079 g, 0.164 mmol) and 2-amino-2-methylpropane-1,3-diol (0.034 g, 0.327 mmol) in dry N,N-dimethylformamide was treated with acetic acid, stirred for 30 minutes, then treated with sodium cyanoborohydride (0.021 g, 0.327 mmol). The mixture was stirred for 3 days. The reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 40-80% B over 15 minutes, then a 5−minute hold at 100% B; Flow: 20 mL/min. Fractions containing the desired product were combined and dried via centrifugal evaporation, affording the product (0.0431 g, 0.075 mmol, 46.1% yield). The estimated purity by LCMS analysis was 100%. LCMS (Condition ACN-AA, ES+) M+H=572.1, 1.71 minutes, calculated exact mass=571.23. ¹H NMR (500 MHz, DMSO-d₆) δ: 7.48 (d, J=7.7 Hz, 1H), 7.38 (s, 1H), 7.28-7.22 (m, 1H), 7.17 (d, J=6.6 Hz, 1H), 6.95-6.90 (m, 2H), 6.76 (d, J=1.8 Hz, 1H), 6.74 (dd, J=8.1, 2.2 Hz, 1H), 5.24 (s, 2H), 4.28 (s, 4H), 4.01 (dd, J=9.4, 5.7 Hz, 1H), 3.91 (dd, J=9.4, 6.1 Hz, 1H), 3.68 (br. s., 1H), 3.45 (dd, J=5.7, 2.8 Hz, 1H), 2.24 (s, 3H), 2.07-1.97 (m, 1H), 1.91 (s, 2H), 1.02-0.97 (m, 6H); partially solvent obscured.

### Intermediate: (S)-5-Chloro-4-((3-(2,3-dihydrobenzo[b][1,4]dioxin-6-yl)-2-methylbenzyl)oxy)-2-(3-hydroxy-2-methylpropoxy)benzaldehyde

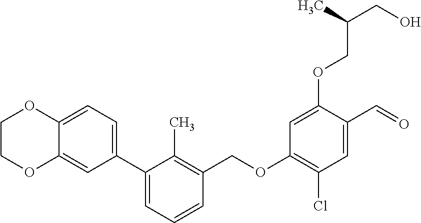 [SMILES: Cc1c(COc2cc(OC[C@@H](C)CO)c(C=O)cc2Cl)cccc1-c1ccc2c(c1)OCCO2]

A suspension of 5-chloro-4-((3-(2,3-dihydrobenzo[b][1,4]dioxin-6-yl)-2-methylbenzyl)oxy)-2-hydroxybenzaldehyde (0.209 g, 0.509 mmol) in dry N,N-dimethylformamide (1.5 mL) was treated with sodium hydride, 60% oil dispersion (0.024 g, 0.610 mmol) and the mixture was stirred for 10 minutes at room temperature, until the suspension became a solution. The reaction was treated with (S)-3-bromo-2-methylpropan-1-ol (0.064 mL, 0.610 mmol) and stirred with heating (50° C. oil bath) for 16 hours. The reaction mixture was diluted with saturated aqueous ammonium chloride and water, and then extracted with ethyl acetate. The organic layer was washed with water, then brine, dried over anhydrous sodium sulphate, filtered, and concentrated under reduced pressure to a viscous yellow oil. The residue was purified by biotage (RediSep 24 g SiO₂, 0% (3 CV), 0-25% (25 CV), 25% (2 CV), methanol in dichloromethane). Product fractions were pooled and concentrated under reduced pressure, affording the product (0.213 g, 0.441 mmol, 87% yield) as a clear viscous oil. LCMS (ES+) M+Na=505.1. The material was used directly in following reactions.

### Example 1034: (S)-1-(5-chloro-4-((3-(2,3-dihydrobenzo[b][1,4]dioxin-6-yl)-2-methylbenzyl)oxy)-2-((S)-3-hydroxy-2-methylpropoxy)benzyl)piperidine-2-carboxylic acid

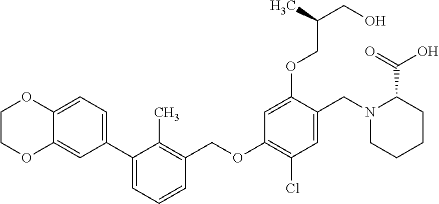 [SMILES: Cc1c(COc2cc(OC[C@@H](C)CO)c(CN3CCCC[C@H]3C(=O)O)cc2Cl)cccc1-c1ccc2c(c1)OCCO2]

A suspension of(S)-5-chloro-4-((3-(2,3-dihydrobenzo[b][1,4]dioxin-6-yl)-2-methylbenzyl)oxy)-2-(3-hydroxy-2-methylpropoxy)benzaldehyde (0.070 g, 0.145 mmol) and (S)-piperidine-2-carboxylic acid (0.037 g, 0.290 mmol) in dry N,N-dimethylformamide (2.0 ml) was treated with acetic acid (0.041 ml, 0.725 mmol), stirred for 30 minutes, then treated with sodium cyanoborohydride (0.018 g, 0.290 mmol). The reaction was stirred for 16 hours. The reaction was diluted with minimal drops of water to bring about dissolution, then the reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 40-80% B over 20 minutes, then a 5-minute hold at 100% B; Flow: 20 mL/min. Fractions containing the desired product were combined and dried via centrifugal evaporation, affording the product (0.0193 g, 0.032 mmol, 22.11% yield). The estimated purity by LCMS analysis was 99%. LCMS (Condition ACN-AA, ES+) M+H=596.3, 1.74 minutes, calculated exact mass=595.23. ¹H NMR (500 MHz, DMSO-d₆) δ: 7.48 (d, J=7.3 Hz, 1H), 7.41 (s, 1H), 7.28-7.22 (m, 1H), 7.18 (d, J=7.3 Hz, 1H), 6.95 (s, 1H), 6.92 (d, J=8.4 Hz, 1H), 6.80-6.71 (m, 2H), 5.24 (s, 2H), 4.28 (s, 4H), 4.05-3.98 (m, 1H), 3.92 (dd, J=9.2, 5.9 Hz, 1H), 3.78 (d, J=13.6 Hz, 1H), 3.69-3.61 (m, 1H), 3.48-3.39 (m, 1H), 3.16 (dd, J=7.9, 3.9 Hz, 1H), 2.94 (d, J=10.3 Hz, 1H), 2.35 (d, J=6.6 Hz, 1H), 2.24 (s, 3H), 2.07-1.97 (m, 1H), 1.83 (br. s., 1H), 1.71 (d, J=9.5 Hz, 1H), 1.50 (br. s., 3H), 1.38 (br. s., 1H), 0.98 (d, J=7.0 Hz, 3H); partially solvent obscured.

### Example 1035: (R)-2-((5-chloro-4-((3-(2,3-dihydrobenzo[b][1,4]dioxin-6-yl)-2-methylbenzyl)oxy)-2-((S)-3-hydroxy-2-methylpropoxy)benzyl)amino)-3-hydroxy-2-methylpropanoic acid

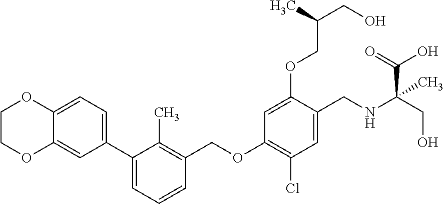 [SMILES: Cc1c(COc2cc(OC[C@@H](C)CO)c(CN[C@](C)(CO)C(=O)O)cc2Cl)cccc1-c1ccc2c(c1)OCCO2]

A suspension of(S)-5-chloro-4-((3-(2,3-dihydrobenzo[b][1,4]dioxin-6-yl)-2-methylbenzyl)oxy)-2-(3-hydroxy-2-methylpropoxy)benzaldehyde (0.067 g, 0.139 mmol) and (R)-2-amino-3-hydroxy-2-methylpropanoic acid (0.033 g, 0.277 mmol) in dry N,N-dimethylformamide (2.0 mL) was treated with acetic acid (0.040 mL, 0.694 mmol), stirred for 30 minutes, then treated with sodium cyanoborohydride (0.017 g, 0.277 mmol). The mixture was stirred for 16 hours. The reaction was diluted with minimal drops of water to bring about dissolution, then the reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 35-75% B over 19 minutes, then a 3-minute hold at 100% B; Flow: 20 mL/min. Fractions containing the desired product were combined and dried via centrifugal evaporation, affording the product (0.0402 g, 0.069 mmol, 49.4% yield). The estimated purity by LCMS analysis was 100%. LCMS (Condition ACN-AA, ES+) M+H=586.1, 1.79 minutes, calculated exact mass=585.21.

### Example 1036: (S)-2-((5-Chloro-4-((3-(2,3-dihydrobenzo[b][1,4]dioxin-6-yl)-2-methylbenzyl)oxy)-2-(3-hydroxy-2-methylpropoxy)benzyl)amino)-2-methylpropane-1,3-diol

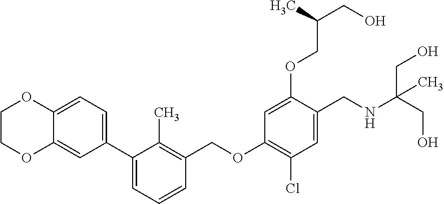 [SMILES: Cc1c(COc2cc(OC[C@@H](C)CO)c(CNC(C)(CO)CO)cc2Cl)cccc1-c1ccc2c(c1)OCCO2]

A solution of(S)-5-chloro-4-((3-(2,3-dihydrobenzo[b][1,4]dioxin-6-yl)-2-methylbenzyl)oxy)-2-(3-hydroxy-2-methylpropoxy)benzaldehyde (0.072 g, 0.149 mmol) and 2-amino-2-methylpropane-1,3-diol (0.031 g, 0.298 mmol) in dry N,N-dimethylformamide (2.0 mL) was treated with acetic acid, stirred for 30 minutes, then treated with sodium cyanoborohydride (0.019 g, 0.298 mmol). The mixture was stirred for 16 hours. The reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 32-72% B over 20 minutes, then a 5-minute hold at 100% B; Flow: 20 mL/min. Fractions containing the desired product were combined and dried via centrifugal evaporation, affording the product (0.0633 g, 0.111 mmol, 74.2% yield). The estimated purity by LCMS analysis was 100%. LCMS (Condition ACN-AA, ES+) M+H=572.2, 1.72 minutes, calculated exact mass=571.23. ¹H NMR (500 MHz, DMSO-d₆) δ: 7.48 (d, J=7.0 Hz, 1H), 7.35 (s, 1H), 7.29-7.22 (m, 1H), 7.17 (d, J=6.6 Hz, 1H), 6.95-6.87 (m, 2H), 6.76 (d, J=1.8 Hz, 1H), 6.75-6.72 (m, 1H), 5.23 (s, 2H), 4.28 (s, 4H), 4.03-3.97 (m, 1H), 3.90 (dd, J=9.2, 5.9 Hz, 1H), 3.60 (s, 1H), 3.45 (t, J=5.9 Hz, 1H), 3.30 (s, 1H), 2.24 (s, 3H), 2.05-1.97 (m, 1H), 1.90 (s, 2H), 0.99 (d, J=7.0 Hz, 3H), 0.95 (s, 3H); partially solvent obscured.

### Intermediate: 5-Chloro-4-((3-(2,3-dihydrobenzo[b][1,4]dioxin-6-yl)-2-methylbenzyl)oxy)-2-(2-morpholinoethoxy)benzaldehyde

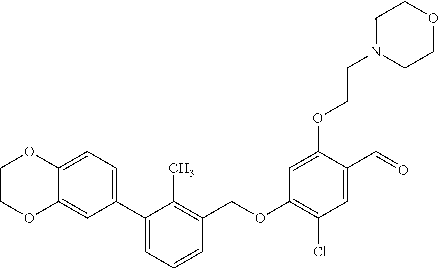 [SMILES: Cc1c(COc2cc(OCCN3CCOCC3)c(C=O)cc2Cl)cccc1-c1ccc2c(c1)OCCO2]

A solution of 2-(2-bromoethoxy)-5-chloro-4-((3-(2,3-dihydrobenzo[b][1,4]dioxin-6-yl)-2-methylbenzyl)oxy)benzaldehyde (0.150 g, 0.290 mmol) in dry N,N-dimethylformamide (2.5 ml) was treated with potassium carbonate (0.080 g, 0.579 mmol) and morpholine (0.126 g, 1.448 mmol). The mixture was stirred with heating (70° C. oil bath) for 16 hours. The reaction was diluted with ethyl acetate (50 mL) and washed with water (50 mL) followed by brine (50 mL). The organic layer was dried over sodium sulfate, filtered, and concentrated under reduced pressure. The residue was purified by biotage (RediSep 12 g SiO₂, 0% (3 CV), 0-25% (15 CV), 25% (2 CV), methanol in dichloromethane). Product fractions were pooled and concentrated under reduced pressure, affording the product (0.130 g, 0.248 mmol, 86% yield). LCMS (ES+) M+H=524.2. ¹H NMR (400 MHz, CDCl₃) δ: 10.30 (s, 1H), 7.89 (s, 1H), 7.46 (dd, J=5.4, 3.6 Hz, 1H), 7.32-7.26 (m, 2H), 6.94 (d, J=8.3 Hz, 1H), 6.85 (d, J=2.0 Hz, 1H), 6.80 (dd, J=8.2, 2.1 Hz, 1H), 6.63 (s, 1H), 5.24 (s, 2H), 4.33 (s, 4H), 4.23 (t, J=5.6 Hz, 2H), 3.77-3.71 (m, 4H), 2.88 (t, J=5.6 Hz, 2H), 2.64-2.57 (m, 4H), 2.31 (s, 3H).

### Example 1037: (R)-2-((5-Chloro-4-((3-(2,3-dihydrobenzo[b][1,4]dioxin-6-yl)-2-methylbenzyl)oxy)-2-(2-morpholinoethoxy)benzyl)amino)-3-hydroxy-2-methylpropanoic acid

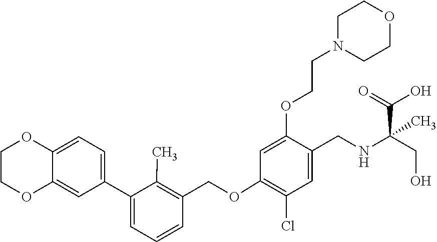 [SMILES: Cc1c(COc2cc(OCCN3CCOCC3)c(CN[C@](C)(CO)C(=O)O)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-(2-morpholinoethoxy)benzaldehyde (0.037 g, 0.071 mmol) and (R)-2-amino-3-hydroxy-2-methylpropanoic acid (0.017 g, 0.141 mmol) in dry N,N-dimethylformamide was treated with acetic acid (0.020 ml, 0.353 mmol), stirred for 30 minutes, then treated with sodium cyanoborohydride (8.87 mg, 0.141 mmol). The mixture was stirred for 16 hours. The reaction was diluted with several drops of water to dissolve solids, filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-70% B over 17 minutes, then a 3-minute hold at 100% B; Flow: 20 m/min. Fractions containing the desired product were combined and dried via centrifugal evaporation, affording the product (0.0284 g, 0.045 mmol, 64.1% yield). The estimated purity by LCMS analysis was 100%. LCMS (Condition ACN-AA, ES+) M+H=627.1, 1.74 minutes, calculated exact mass=626.239. ¹H NMR (500 MHz, DMSO-d₆) δ 7.51-7.43 (m, 2H), 7.26 (t, J=7.5 Hz, 1H), 7.17 (d, J=7.7 Hz, 1H), 7.02 (s, 1H), 6.92 (d, J=8.1 Hz, 1H), 6.80-6.71 (m, 2H), 5.28 (s, 2H), 4.28 (s, 4H), 4.20 (t, J=5.7 Hz, 2H), 3.91 (br. s., 2H), 3.68-3.51 (m, 4H), 2.75 (t, J=5.7 Hz, 2H), 2.48 (br. s., 4H), 2.24 (s, 3H), 1.90 (s, 1H), 1.27 (s, 3H).

### Example 1038: (S)-1-(5-Chloro-4-((3-(2,3-dihydrobenzo[b][1,4]dioxin-6-yl)-2-methylbenzyl)oxy)-2-(2-morpholinoethoxy)benzyl)piperidine-2-carboxylic acid

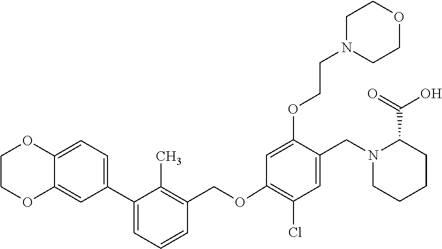 [SMILES: Cc1c(COc2cc(OCCN3CCOCC3)c(CN3CCCC[C@H]3C(=O)O)cc2Cl)cccc1-c1ccc2c(c1)OCCO2]

A suspension of 5-chloro-4-((3-(2,3-dihydrobenzo[b][1,4]dioxin-6-yl)-2-methylbenzyl)oxy)-2-(2-morpholinoethoxy)benzaldehyde (0.038 g, 0.073 mmol) and (S)-piperidine-2-carboxylic acid (0.019 g, 0.145 mmol) in dry N,N-dimethylformamide (1.066 ml) was treated with acetic acid (0.021 ml, 0.363 mmol), stirred for 30 minutes, then treated with sodium cyanoborohydride (9.11 mg, 0.145 mmol). The reaction was stirred for 16 hours. The reaction was diluted with minimal drops of water to bring about dissolution, then filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 35-85% B over 20 minutes, then a 3-minute hold at 100% B; Flow: 20 m/min. Fractions containing the desired product were combined and dried via centrifugal evaporation, affording the product (0.0254 g, 0.040 mmol, 55.0% yield). The estimated purity by LCMS analysis was 100%. LCMS (Condition ACN-AA, ES+) M+H=637.1, 1.81 minutes, calculated exact mass=636.260. ¹H NMR (500 MHz, DMSO-d₆) δ: 7.47 (d, J=7.3 Hz, 1H), 7.43 (s, 1H), 7.26 (t, J=7.5 Hz, 1H), 7.17 (d, J=7.3 Hz, 1H), 6.98 (s, 1H), 6.92 (d, J=8.4 Hz, 1H), 6.79-6.72 (m, 2H), 5.25 (s, 2H), 4.27 (s, 4H), 4.17 (t, J=5.5 Hz, 2H), 3.90-3.66 (m, 2H), 3.57 (t, J=4.6 Hz, 2H), 3.44 (br. s., 1H), 3.19-3.13 (m, 1H), 3.02-2.93 (m, 1H), 2.71 (t, J=5.5 Hz, 2H), 2.48 (br. s., 4H), 2.45-2.38 (m, 1H), 2.24 (s, 3H), 1.90 (s, 1H), 1.89-1.80 (m, 1H), 1.71 (d, J=10.3 Hz, 1H), 1.53 (br. s., 3H), 1.43-1.28 (m, 1H); some peaks were solvent obscured.

### Example 1039: 2-((5-Chloro-4-((3-(2,3-dihydrobenzo[b][1,4]dioxin-6-yl)-2-methylbenzyl)oxy)-2-(2-morpholinoethoxy)benzyl)amino)-2-methylpropane-1,3-diol

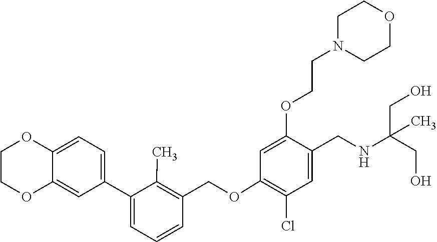 [SMILES: Cc1c(COc2cc(OCCN3CCOCC3)c(CNC(C)(CO)CO)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-(2-morpholinoethoxy)benzaldehyde (0.044 g, 0.084 mmol) and 2-amino-2-methylpropane-1,3-diol (0.018 g, 0.168 mmol) in dry N,N-dimethylformamide (1.235 ml) was treated with acetic acid, stirred for 30 minutes, then treated with sodium cyanoborohydride (10.55 mg, 0.168 mmol). The mixture was stirred for 16 hours. The reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 32-72% B over 17 minutes, then a 3-minute hold at 100% B; Flow: 20 mL/min. Fractions containing the desired product were combined and dried via centrifugal evaporation, affording the product (0.0472 g, 0.077 mmol, 92% yield). The estimated purity by LCMS analysis was 100%. LCMS (Condition ACN-AA, ES+) M+H=613.2, 1.99 minutes, calculated exact mass=612.260. ¹H NMR (500 MHz, DMSO-d₆) δ: 7.46 (d, J=7.7 Hz, 1H), 7.35 (s, 1H), 7.25 (t, J=7.5 Hz, 1H), 7.16 (d, J=7.3 Hz, 1H), 6.94 (s, 1H), 6.92 (d, J=8.1 Hz, 1H), 6.78-6.70 (m, 2H), 5.24 (s, 2H), 4.27 (s, 4H), 4.15 (t, J=5.5 Hz, 2H), 3.61 (s, 1H), 3.58 (t, J=4.6 Hz, 2H), 3.31 (s, 2H), 2.71 (t, J=5.5 Hz, 2H), 2.48 (br. s., 4H), 2.23 (s, 3H), 1.90 (s, 2H), 0.96 (s, 3H); several peaks were solvent obscured.

### Intermediate: 5-Chloro-2-(cyclohexylmethoxy)-4-((3-(2,3-dihydrobenzo[b][1,4]dioxin-6-yl)-2-methylbenzyl)oxy)benzaldehyde

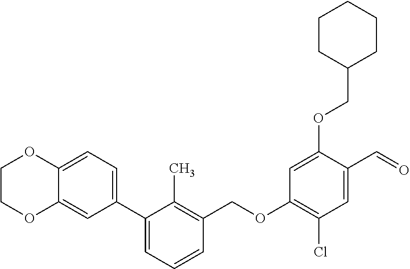 [SMILES: Cc1c(COc2cc(OCC3CCCCC3)c(C=O)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-hydroxybenzaldehyde (0.211 g, 0.514 mmol) in dry N,N-dimethylformamide (5.0 mL) was treated with cesium carbonate (0.251 g, 0.770 mmol) followed by (bromomethyl)cyclohexane (0.455 g, 2.57 mmol). The mixture was stirred with heating (70° C. oil bath) for 3.5 hours. The reaction was cooled and stirred for 16 hours. The reaction was diluted with ethyl acetate (15 mL) and washed with water (2×20 mL) followed by brine (20 mL). The organic layer was dried over sodium sulfate, filtered, and concentrated under reduced pressure. The residue was purified by biotage (RediSep 12 g SiO₂, 0% (3 CV), 0-50% (15 CV), 50% (2 CV), ethyl acetate in hexanes). Product fractions were pooled and concentrated under reduced pressure, affording the product (0.243 g, 0.479 mmol, 93% yield) as a clear oil that slowly solidified upon standing. LCMS (ES+) M+H=507.1. ¹H NMR (400 MHz, CDCl₃) δ: 10.32 (s, 1H), 7.87 (s, 1H), 7.49-7.41 (m, 1H), 7.27-7.22 (m, 2H), 6.92 (d, J=8.3 Hz, 1H), 6.83 (d, J=2.0 Hz, 1H), 6.78 (dd, J=8.3, 2.3 Hz, 1H), 6.57 (s, 1H), 5.23 (s, 2H), 4.32 (s, 4H), 3.86 (d, J=5.8 Hz, 2H), 2.31 (s, 3H), 1.94-1.68 (m, 6H), 1.59 (s, 1H), 1.38-1.19 (m, 4H), 1.18-1.03 (m, 2H).

### Example 1040: (S)-1-(5-Chloro-2-(cyclohexylmethoxy)-4-((3-(2,3-dihydrobenzo[b][1,4]dioxin-6-yl)-2-methylbenzyl)oxy)benzyl)piperidine-2-carboxylic acid

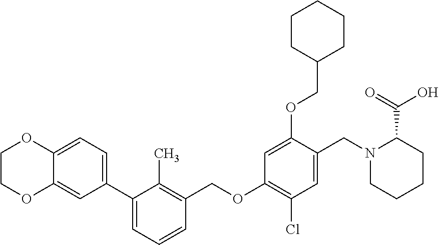 [SMILES: Cc1c(COc2cc(OCC3CCCCC3)c(CN3CCCC[C@H]3C(=O)O)cc2Cl)cccc1-c1ccc2c(c1)OCCO2]

A suspension of 5-chloro-2-(cyclohexylmethoxy)-4-((3-(2,3-dihydrobenzo[b][1,4]dioxin-6-yl)-2-methylbenzyl)oxy)benzaldehyde (0.074 g, 0.146 mmol) and (S)-piperidine-2-carboxylic acid (0.038 g, 0.292 mmol) in dry N,N-dimethylformamide (2.146 ml) was treated with acetic acid (0.042 ml, 0.730 mmol), stirred for 30 minutes, then treated with sodium cyanoborohydride (0.018 g, 0.292 mmol). The reaction was stirred for 16 hours. The reaction was diluted with minimal drops of water to bring about dissolution, then filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 45-85% B over 17 minutes, then a 3-minute hold at 100% B; Flow: 20 m/min. Fractions containing the desired product were combined and dried via centrifugal evaporation, affording the product (0.0213 g, 0.034 mmol, 23.53% yield). The estimated purity by LCMS analysis was 100%. LCMS (Condition ACN-AA, ES+) M+H=620.2, 2.31 minutes, calculated exact mass=619.270. ¹H NMR (500 MHz, DMSO-d₆) δ: 7.46 (d, J=7.7 Hz, 1H), 7.42 (s, 1H), 7.25 (t, J=7.5 Hz, 1H), 7.17 (d, J=7.3 Hz, 1H), 6.94-6.87 (m, 2H), 6.78-6.71 (m, 2H), 5.24 (s, 2H), 4.27 (s, 4H), 3.85 (d, J=5.9 Hz, 2H), 3.81-3.58 (m, 2H), 3.18-3.10 (m, 1H), 2.91 (br. s., 1H), 2.33 (br. s., 1H), 2.24 (s, 3H), 1.87-1.59 (m, 8H), 1.50 (br. s., 3H), 1.37 (br. s., 1H), 1.31-0.98 (m, 5H).

### Example 1041: 2-((5-Chloro-2-(cyclohexylmethoxy)-4-((3-(2,3-dihydrobenzo[b][1,4]dioxin-6-yl)-2-methylbenzyl)oxy)benzyl)amino)-2-methylpropane-1,3-diol

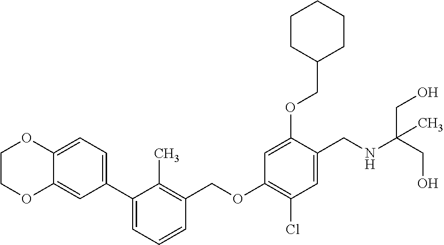 [SMILES: Cc1c(COc2cc(OCC3CCCCC3)c(CNC(C)(CO)CO)cc2Cl)cccc1-c1ccc2c(c1)OCCO2]

A solution of 5-chloro-2-(cyclohexylmethoxy)-4-((3-(2,3-dihydrobenzo[b][1,4]dioxin-6-yl)-2-methylbenzyl)oxy)benzaldehyde (0.065 g, 0.128 mmol) and 2-amino-2-methylpropane-1,3-diol (0.027 g, 0.256 mmol) in dry N,N-dimethylformamide (1.885 ml) was treated with acetic acid, stirred for 30 minutes, then treated with sodium cyanoborohydride (0.016 g, 0.256 mmol). The mixture was stirred for 16 hours. The reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 48-88% B over 15 minutes, then a 4-minute hold at 100% B; Flow: 20 mL/min. Fractions containing the desired product were combined and dried via centrifugal evaporation, affording the product. The estimated purity by LCMS analysis was 100%. LCMS (Condition ACN-AA, ES+) M+H=596.2, 2.46 minutes, calculated exact mass=595.270. ¹H NMR (500 MHz, DMSO-d₆) δ: 7.45 (d, J=7.0 Hz, 1H), 7.32 (s, 1H), 7.24 (t, J=7.5 Hz, 1H), 7.16 (d, J=7.3 Hz, 1H), 6.92 (d, J=8.1 Hz, 1H), 6.86 (s, 1H), 6.78-6.70 (m, 2H), 5.22 (s, 2H), 4.27 (s, 4H), 3.83 (d, J=5.9 Hz, 2H), 3.56 (s, 1H), 3.27 (s, 2H), 2.23 (s, 3H), 1.88 (s, 2H), 1.81 (d, J=11.7 Hz, 2H), 1.71 (d, J=13.6 Hz, 3H), 1.65 (d, J=12.1 Hz, 1H), 1.32-1.03 (m, 5H), 0.92 (s, 3H); some signals were solvent obscured.

### Intermediate: 5-Chloro-4-((3-(2,3-dihydrobenzo[b][1,4]dioxin-6-yl)-2-methylbenzyl)oxy)-2-(2-(piperidin-1-yl)ethoxy)benzaldehyde

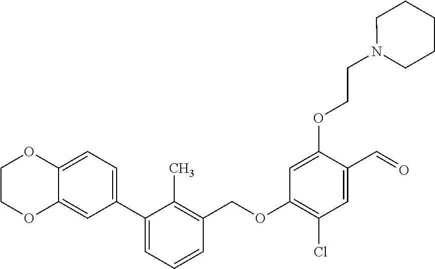 [SMILES: Cc1c(COc2cc(OCCN3CCCCC3)c(C=O)cc2Cl)cccc1-c1ccc2c(c1)OCCO2]

A solution of 2-(2-bromoethoxy)-5-chloro-4-((3-(2,3-dihydrobenzo[b][1,4]dioxin-6-yl)-2-methylbenzyl)oxy)benzaldehyde (0.140 g, 0.270 mmol) in dry N,N-dimethylformamide (2.5 ml) was treated with potassium carbonate (0.075 g, 0.541 mmol) and piperidine (0.115 g, 1.352 mmol). The mixture was stirred with heating (70° C. oil bath) for 16 hours The reaction was diluted with ethyl acetate (20 mL) and washed with water (20 mL) followed by brine (20 mL). The organic layer was dried over sodium sulfate, filtered, and concentrated under reduced pressure. The residue was purified by biotage (RediSep 12 g SiO₂, 0% (3 CV), 0-25% (15 CV), 25% (2 CV), methanol in dichloromethane). Product fractions were pooled and concentrated under reduced pressure, affording the product (0.105 g, 0.201 mmol, 74.4% yield) as a viscous oil that crystallized slowly upon standing under vacuum. LCMS (ES+) M+H=522.1. ¹H NMR (500 MHz, CDCl₃) δ: 10.30 (s, 1H), 7.89 (s, 1H), 7.47 (dd, J=6.1, 2.7 Hz, 1H), 7.31-7.27 (m, 2H), 6.94 (d, J=8.2 Hz, 1H), 6.85 (d, J=2.1 Hz, 1H), 6.80 (dd, J=8.2, 2.1 Hz, 1H), 6.66 (s, 1H), 5.23 (s, 2H), 4.33 (s, 4H), 4.23 (t, J=6.0 Hz, 2H), 2.85 (t, J=6.0 Hz, 2H), 2.54 (br. s., 4H), 2.31 (s, 3H), 1.62 (quin, J=5.6 Hz, 4H), 1.51-1.44 (m, 2H).

### Example 1042: (S)-1-(5-Chloro-4-((3-(2,3-dihydrobenzo[b][1,4]dioxin-6-yl)-2-methylbenzyl)oxy)-2-(2-(piperidin-1-yl)ethoxy)benzyl)piperidine-2-carboxylic acid

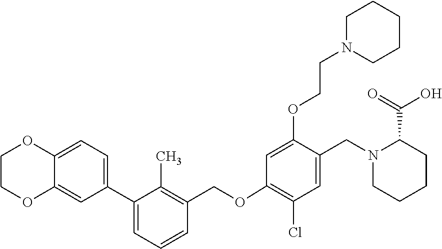 [SMILES: Cc1c(COc2cc(OCCN3CCCCC3)c(CN3CCCC[C@H]3C(=O)O)cc2Cl)cccc1-c1ccc2c(c1)OCCO2]

A suspension of 5-chloro-4-((3-(2,3-dihydrobenzo[b][1,4]dioxin-6-yl)-2-methylbenzyl)oxy)-2-(2-(piperidin-1-yl)ethoxy)benzaldehyde (0.033 g, 0.063 mmol) and (S)-piperidine-2-carboxylic acid (0.016 g, 0.126 mmol) in dry N,N-dimethylformamide (0.930 ml) was treated with acetic acid (0.018 ml, 0.316 mmol), stirred for 30 minutes, then treated with sodium cyanoborohydride (7.95 mg, 0.126 mmol). The reaction was stirred for 16 hours. The reaction was diluted with minimal drops of water to bring about dissolution, then filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 25-80% B over 25 minutes, then a 5-minute hold at 100% B; Flow: 20 mL/min. Fractions containing the desired product were combined and dried via centrifugal evaporation, affording the product (0.0051 g, 7.55 μmol, 11.94% yield). The estimated purity by LCMS analysis was 94%. LCMS (Condition ACN-AA, ES+) M+H=635.0, 1.61 minutes, calculated exact mass=634.281.

### Example 1043: (R)-2-((5-Chloro-4-((3-(2,3-dihydrobenzo[b][1,4]dioxin-6-yl)-2-methylbenzyl)oxy)-2-(2-(piperidin-1-yl)ethoxy)benzyl)amino)-3-hydroxy-2-methylpropanoic acid

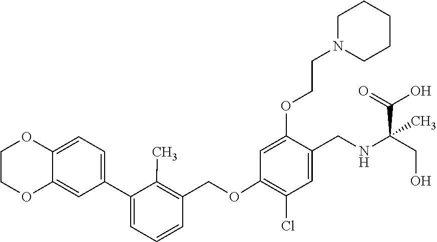 [SMILES: Cc1c(COc2cc(OCCN3CCCCC3)c(CN[C@](C)(CO)C(=O)O)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-(2-(piperidin-1-yl)ethoxy)benzaldehyde (0.032 g, 0.061 mmol) and (R)-2-amino-3-hydroxy-2-methylpropanoic acid (0.015 g, 0.123 mmol) in dry N,N-dimethylformamide (0.901 ml) was treated with acetic acid (0.018 ml, 0.306 mmol), stirred for 30 minutes, then treated with sodium cyanoborohydride (7.70 mg, 0.123 mmol). The mixture was stirred for 16 hours. The reaction was diluted with several drops of water to dissolve solids, filtered (0.45 m syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-80% B over 25 minutes, then a 5-minute hold at 100% B; Flow: 20 mL/min. Fractions containing the desired product were combined and dried via centrifugal evaporation, affording the product (0.0012 g, 1.900 μmol, 3.10% yield). The estimated purity by LCMS analysis was 99%. LCMS (Condition ACN-AA, ES+) M+H=625.1, 1.56 minutes, calculated exact mass=624.260.

### Example 1044: 2-((5-Chloro-4-((3-(2,3-dihydrobenzo[b][1,4]dioxin-6-yl)-2-methylbenzyl)oxy)-2-(2-(piperidin-1-yl)ethoxy)benzyl)amino)-2-methylpropane-1,3-diol

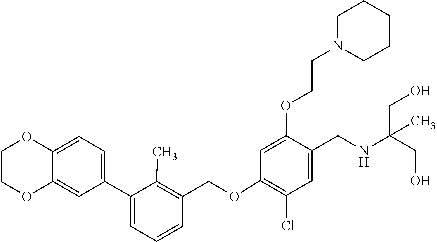 [SMILES: Cc1c(COc2cc(OCCN3CCCCC3)c(CNC(C)(CO)CO)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-(2-(piperidin-1-yl)ethoxy)benzaldehyde (0.033 g, 0.063 mmol) and 2-amino-2-methylpropane-1,3-diol (0.013 g, 0.126 mmol) in dry N,N-dimethylformamide (0.930 ml) was treated with acetic acid, stirred for 30 minutes, then treated with sodium cyanoborohydride (7.95 mg, 0.126 mmol). The mixture was stirred for 16 hours. The reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 40-80% B over 20 minutes, then a 5-minute hold at 100% B; Flow: 20 mL/min. Fractions containing the desired product were combined and dried via centrifugal evaporation, affording the product (0.0366 g, 0.060 mmol, 95% yield). The estimated purity by LCMS analysis was 100%. LCMS (Condition ACN-AA, ES+) M+H=611.1, 1.90 minutes, calculated exact mass=610.281. ¹H NMR (500 MHz, DMSO-d₆) δ: 7.47 (d, J=7.0 Hz, 1H), 7.37 (s, 1H), 7.25 (t, J=7.5 Hz, 1H), 7.17 (d, J=7.0 Hz, 1H), 6.97 (s, 1H), 6.92 (d, J=8.1 Hz, 1H), 6.79-6.71 (m, 2H), 5.26 (s, 2H), 4.28 (s, 4H), 4.15 (t, J=5.7 Hz, 2H), 3.71 (br. s., 1H), 2.69 (t, J=5.5 Hz, 2H), 2.45 (br. s., 3H), 2.24 (s, 3H), 1.90 (s, 3H), 1.51 (quin, J=5.5 Hz, 4H), 1.38 (br. s., 2H), 1.01 (s, 3H); several peaks are solvent obscured.

### Intermediate: 5-Chloro-4-((3-(2,3-dihydrobenzo[b][1,4]dioxin-6-yl)-2-methylbenzyl)oxy)-2-((tetrahydro-2H-pyran-4-yl)methoxy)benzaldehyde

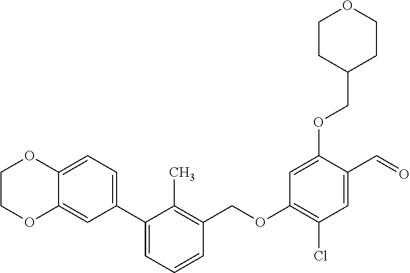 [SMILES: Cc1c(COc2cc(OCC3CCOCC3)c(C=O)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-hydroxybenzaldehyde (0.148 g, 0.360 mmol) in dry N,N-dimethylformamide (3.5 mL) was treated with cesium carbonate (0.176 g, 0.540 mmol) followed by 4-(bromomethyl)tetrahydro-2H-pyran (0.132 g, 0.737 mmol). The mixture was stirred with heating (70° C. oil bath) for 20 hours. The reaction was diluted with ethyl acetate (20 mL) and washed with water (20 mL) followed by brine (20 mL). The organic layer was dried over sodium sulfate, filtered, and concentrated under reduced pressure. The residue was purified by biotage (RediSep 12 g SiO₂, 0% (3 CV), 0-20% (15 CV), 20% (2 CV), methanol in dichloromethane). Product fractions were pooled and concentrated under reduced pressure, affording the product (0.179 g, 0.352 mmol, 98% yield) as a pale orange solid. LCMS (ES+) M+H=509.1. ¹H NMR (400 MHz, CDCl₃) δ: 10.29 (s, 1H), 7.86 (s, 1H), 7.43 (t, J=4.5 Hz, 1H), 7.26-7.21 (m, 2H), 6.91 (d, J=8.3 Hz, 1H), 6.82 (d, J=2.0 Hz, 1H), 6.77 (dd, J=8.3, 2.0 Hz, 1H), 6.57 (s, 1H), 5.23 (s, 2H), 4.31 (s, 4H), 4.04 (dd, J=11.3, 3.3 Hz, 2H), 3.90 (d, J=6.5 Hz, 2H), 3.46 (td, J=11.8, 2.0 Hz, 2H), 2.30 (s, 3H), 2.21-2.07 (m, 1H), 1.74 (br. s., 2H), 1.57-1.43 (m, 2H).

### Example 1045: (R)-2-((5-Chloro-4-((3-(2,3-dihydrobenzo[b][1,4]dioxin-6-yl)-2-methylbenzyl)oxy)-2-((tetrahydro-2H-pyran-4-yl)methoxy)benzyl)amino)-3-hydroxy-2-methylpropanoic acid

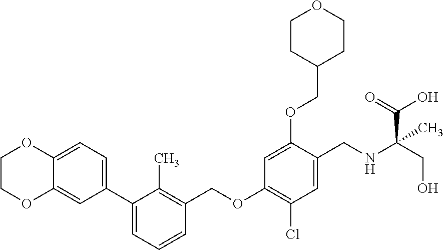 [SMILES: Cc1c(COc2cc(OCC3CCOCC3)c(CN[C@](C)(CO)C(=O)O)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-((tetrahydro-2H-pyran-4-yl)methoxy)benzaldehyde (0.033 g, 0.065 mmol) and (R)-2-amino-3-hydroxy-2-methylpropanoic acid (0.015 g, 0.130 mmol) in dry N,N-dimethylformamide (0.953 ml) was treated with acetic acid (0.019 ml, 0.324 mmol), stirred for 30 minutes, then treated with sodium cyanoborohydride (8.15 mg, 0.130 mmol). The mixture was stirred for 16 hours. The reaction was diluted with several drops of water to dissolve solids, filtered (0.45 m syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 38-90% B over 22 minutes, then a 5-minute hold at 100% B; Flow: 20 mL/min. Fractions containing the desired product were combined and dried via centrifugal evaporation, affording the product (0.0078 g, 0.013 mmol, 19.46% yield). The estimated purity by LCMS analysis was 99%. LCMS (Condition ACN-AA, ES+) M+H=612.1, 1.74 minutes, calculated exact mass=611.229. ¹H NMR (500 MHz, DMSO-d₆) δ: 7.51-7.44 (m, 2H), 7.26 (t, J=7.5 Hz, 1H), 7.18 (d, J=7.0 Hz, 1H), 6.98 (s, 1H), 6.92 (d, J=8.1 Hz, 1H), 6.79-6.70 (m, 2H), 5.28 (s, 2H), 4.28 (s, 4H), 3.94 (dd, J=6.6, 2.9 Hz, 2H), 3.91-3.84 (m, 4H), 3.65-3.48 (m, 1H), 2.24 (s, 3H), 2.14-2.01 (m, 1H), 1.74 (d, J=12.5 Hz, 2H), 1.40-1.27 (m, 2H), 1.26 (s, 3H); several peaks were solvent obscured.

### Example 1046: (S)-1-(5-Chloro-4-((3-(2,3-dihydrobenzo[b][1,4]dioxin-6-yl)-2-methylbenzyl)oxy)-2-((tetrahydro-2H-pyran-4-yl)methoxy)benzyl)piperidine-2-carboxylic acid

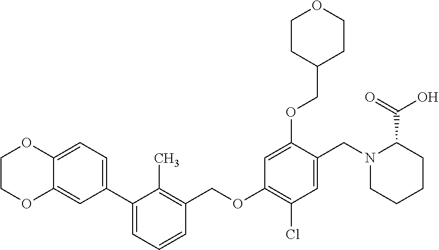 [SMILES: Cc1c(COc2cc(OCC3CCOCC3)c(CN3CCCC[C@H]3C(=O)O)cc2Cl)cccc1-c1ccc2c(c1)OCCO2]

A suspension of 5-chloro-4-((3-(2,3-dihydrobenzo[b][1,4]dioxin-6-yl)-2-methylbenzyl)oxy)-2-((tetrahydro-2H-pyran-4-yl)methoxy)benzaldehyde (0.034 g, 0.067 mmol) and (S)-piperidine-2-carboxylic acid (0.017 g, 0.134 mmol) in dry N,N-dimethylformamide (0.982 ml) was treated with acetic acid (0.019 ml, 0.334 mmol), stirred for 30 minutes, then treated with sodium cyanoborohydride (8.40 mg, 0.134 mmol). The reaction was stirred for 16 hours. The reaction was diluted with minimal drops of water to bring about dissolution, then the reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 42-82% B over 17 minutes, then a 3-minute hold at 100% B; Flow: 20 mL/min. Fractions containing the desired product were combined and dried via centrifugal evaporation, affording the product (0.0045 g, 7.23 μmol, 10.83% yield). The estimated purity by LCMS analysis was 100%. LCMS (Condition ACN-AA, ES+) M+H=622.2, 1.87 minutes, calculated exact mass=621.249. ¹H NMR (500 MHz, DMSO-d₆) δ: 7.47 (d, J=7.7 Hz, 1H), 7.43 (s, 1H), 7.26 (t, J=7.5 Hz, 1H), 7.17 (d, J=7.3 Hz, 1H), 6.95-6.89 (m, 2H), 6.77 (d, J=2.2 Hz, 1H), 6.76-6.72 (m, 1H), 5.24 (s, 2H), 4.28 (s, 4H), 3.92-3.83 (m, 4H), 3.77-3.54 (m, 1H), 3.38-3.29 (m, 1H), 3.09 (d, J=4.0 Hz, 1H), 2.90 (d, J=11.4 Hz, 1H), 2.26 (br. s., 1H), 2.24 (s, 3H), 2.01 (br. s., 1H), 1.79 (br. s., 1H), 1.69 (d, J=13.2 Hz, 3H), 1.49 (br. s., 3H), 1.40-1.28 (m, 3H).

### Example 1047: 2-((5-Chloro-4-((3-(2,3-dihydrobenzo[b][1,4]dioxin-6-yl)-2-methylbenzyl)oxy)-2-((tetrahydro-2H-pyran-4-yl)methoxy)benzyl)amino)-2-methylpropane-1,3-diol

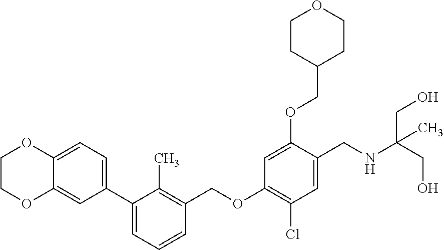 [SMILES: Cc1c(COc2cc(OCC3CCOCC3)c(CNC(C)(CO)CO)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-((tetrahydro-2H-pyran-4-yl)methoxy)benzaldehyde (0.031 g, 0.061 mmol) and 2-amino-2-methylpropane-1,3-diol (0.013 g, 0.122 mmol) in dry N,N-dimethylformamide (0.896 ml) was treated with acetic acid, stirred for 30 minutes, then treated with sodium cyanoborohydride (7.65 mg, 0.122 mmol). The mixture was stirred for 16 hours. The reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 40-80% B over 17 minutes, then a 3-minute hold at 100% B; Flow: 20 mL/min. Fractions containing the desired product were combined and dried via centrifugal evaporation, affording the product (0.0287 g, 0.048 mmol, 78.8% yield). The estimated purity by LCMS analysis was 100%. LCMS (Condition ACN-AA, ES+) M+H=598.1, 1.81 minutes, calculated exact mass=597.249. ¹H NMR (500 MHz, DMSO-d₆) δ: 7.47 (d, J=6.6 Hz, 1H), 7.34 (s, 1H), 7.25 (t, J=7.5 Hz, 1H), 7.17 (d, J=6.6 Hz, 1H), 6.92 (d, J=8.1 Hz, 1H), 6.89 (s, 1H), 6.76 (d, J=1.8 Hz, 1H), 6.75-6.71 (m, 1H), 5.23 (s, 2H), 4.28 (s, 4H), 3.93-3.83 (m, 4H), 3.56 (s, 1H), 3.27 (s, 2H), 2.24 (s, 3H), 2.01 (br. s., 1H), 1.89 (s, 2H), 1.71 (d, J=12.8 Hz, 2H), 1.36 (qd, J=12.3, 4.8 Hz, 2H), 0.92 (s, 3H); several peaks were solvent obscured.

### Intermediate: 5-chloro-2-(cyclobutylmethoxy)-4-((3-(2,3-dihydrobenzo[b][1,4]dioxin-6-yl)-2-methylbenzyl)oxy)benzaldehyde

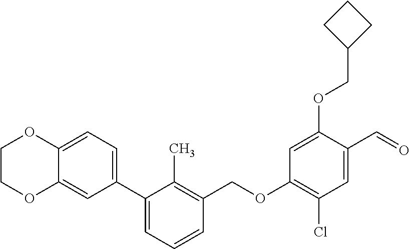 [SMILES: Cc1c(COc2cc(OCC3CCC3)c(C=O)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-hydroxybenzaldehyde (0.210 g, 0.511 mmol) in dry N,N-dimethylformamide (5.0 mL) was treated with cesium carbonate (0.250 g, 0.767 mmol) followed by (bromomethyl)cyclobutane (0.381 g, 2.56 mmol). The mixture was stirred with heating (70° C. oil bath) for 3.5 hours. The reaction was diluted with ethyl acetate (20 mL) and washed with water (20 mL) followed by brine (20 mL). The organic layer was dried over sodium sulfate, filtered, and concentrated under reduced pressure. The residue was purified by biotage (RediSep 12 g SiO₂, 0% (3 CV), 0-25% (20 CV), 25% (2 CV), methanol in dichloromethane). Product fractions were pooled and concentrated under reduced pressure, affording the product (0.210 g, 0.438 mmol, 86% yield) as a white solid. LCMS (ES+) M+H=479.1. ¹H NMR (400 MHz, CDCl₃) δ: 10.32 (s, 1H), 7.87 (s, 1H), 7.49-7.42 (m, 1H), 7.27-7.24 (m, 2H), 6.92 (d, J=8.3 Hz, 1H), 6.83 (d, J=2.3 Hz, 1H), 6.78 (dd, J=8.2, 2.1 Hz, 1H), 6.59 (s, 1H), 5.24 (s, 2H), 4.32 (s, 4H), 4.03 (d, J=6.5 Hz, 2H), 2.91-2.74 (m, 1H), 2.31 (s, 3H), 2.21-2.12 (m, 2H), 2.03-1.85 (m, 4H).

### Example 1048: (R)-2-((5-chloro-2-(cyclobutylmethoxy)-4-((3-(2,3-dihydrobenzo[b][1,4]dioxin-6-yl)-2-methylbenzyl)oxy)benzyl)amino)-3-hydroxy-2-methylpropanoic acid

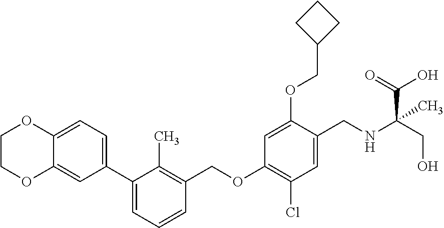 [SMILES: Cc1c(COc2cc(OCC3CCC3)c(CN[C@](C)(CO)C(=O)O)cc2Cl)cccc1-c1ccc2c(c1)OCCO2]

A solution of 5-chloro-2-(cyclobutylmethoxy)-4-((3-(2,3-dihydrobenzo[b][1,4]dioxin-6-yl)-2-methylbenzyl)oxy)benzaldehyde (0.040 g, 0.084 mmol) and (R)-2-amino-3-hydroxy-2-methylpropanoic acid (0.020 g, 0.167 mmol) in dry N,N-dimethylformamide (1.228 ml) was treated with acetic acid (0.024 ml, 0.418 mmol), stirred for 30 minutes, then treated with sodium cyanoborohydride (10.50 mg, 0.167 mmol). The mixture was stirred for 16 hours. The reaction was diluted with several drops of water to dissolve solids, filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 45-85% B over 15 minutes, then a 5-minute hold at 100% B; Flow: 20 mL/min. Fractions containing the desired product were combined and dried via centrifugal evaporation, affording (0.0087 g, 0.014 mmol, 17.36% yield). The estimated purity by LCMS analysis was 97%. LCMS (Condition ACN-AA, ES+) M+H=582.0, 1.83 minutes, calculated exact mass=581.218. ¹H NMR (500 MHz, DMSO-d₆) δ: 7.48 (d, J=7.3 Hz, 1H), 7.47 (s, 1H), 7.29-7.23 (m, 1H), 7.18 (d, J=6.6 Hz, 1H), 6.98 (s, 1H), 6.92 (d, J=8.1 Hz, 1H), 6.77 (d, J=2.2 Hz, 1H), 6.75-6.72 (m, 1H), 5.28 (s, 2H), 4.28 (s, 4H), 4.06 (d, J=6.6 Hz, 2H), 3.89 (s, 2H), 3.64-3.50 (m, 1H), 2.82-2.71 (m, 1H), 2.24 (s, 3H), 2.08 (d, J=7.3 Hz, 2H), 1.96-1.82 (m, 4H), 1.26 (s, 3H), some peaks were solvent obscured.

### Example 1049: 2-((5-Chloro-2-(cyclobutylmethoxy)-4-((3-(2,3-dihydrobenzo[b][1,4]dioxin-6-yl)-2-methylbenzyl)oxy)benzyl)amino)-2-methylpropane-1,3-diol

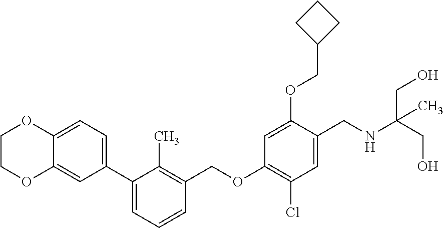 [SMILES: Cc1c(COc2cc(OCC3CCC3)c(CNC(C)(CO)CO)cc2Cl)cccc1-c1ccc2c(c1)OCCO2]

A solution of 5-chloro-2-(cyclobutylmethoxy)-4-((3-(2,3-dihydrobenzo[b][1,4]dioxin-6-yl)-2-methylbenzyl)oxy)benzaldehyde (0.042 g, 0.088 mmol) and 2-amino-2-methylpropane-1,3-diol (0.018 g, 0.175 mmol) in dry N,N-dimethylformamide (1.290 ml) was treated with acetic acid, stirred for 30 minutes, then treated with sodium cyanoborohydride (0.011 g, 0.175 mmol). The mixture was stirred for 16 hours. The reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 45-85% B over 15 minutes, then a 5-minute hold at 100% B; Flow: 20 mL/min. Fractions containing the desired product were combined and dried via centrifugal evaporation, affording the product (0.0407 g, 0.072 mmol, 82% yield). The estimated purity by LCMS analysis was 100%. LCMS (Condition ACN-AA, ES+) M+H=568.1, 1.96 minutes, calculated exact mass=567.239. ¹H NMR (500 MHz, DMSO-d₆) δ: 7.47 (d, J=7.0 Hz, 1H), 7.34 (s, 1H), 7.25 (t, J=7.7 Hz, 1H), 7.17 (d, J=7.3 Hz, 1H), 6.95-6.89 (m, 2H), 6.77 (d, J=1.8 Hz, 1H), 6.74 (dd, J=8.1, 2.2 Hz, 1H), 5.23 (s, 2H), 4.28 (s, 4H), 4.00 (d, J=6.2 Hz, 2H), 3.58 (s, 2H), 2.78-2.65 (m, 1H), 2.24 (s, 3H), 2.13-2.01 (m, 2H), 1.95-1.84 (m, 6H), 0.94 (s, 3H), some peaks were solvent obscured.

### Example 1050: (S)-1-(5-Chloro-2-(cyclobutylmethoxy)-4-((3-(2,3-dihydrobenzo[b][1,4]dioxin-6-yl)-2-methylbenzyl)oxy)benzyl)piperidine-2-carboxylic acid

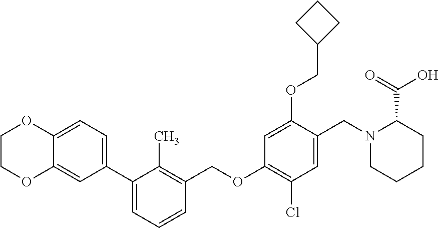 [SMILES: Cc1c(COc2cc(OCC3CCC3)c(CN3CCCC[C@H]3C(=O)O)cc2Cl)cccc1-c1ccc2c(c1)OCCO2]

A suspension of 5-chloro-2-(cyclobutylmethoxy)-4-((3-(2,3-dihydrobenzo[b][1,4]dioxin-6-yl)-2-methylbenzyl)oxy)benzaldehyde (0.041 g, 0.086 mmol) and (S)-piperidine-2-carboxylic acid (0.022 g, 0.171 mmol) in dry N,N-dimethylformamide (1.259 ml) was treated with acetic acid (0.025 ml, 0.428 mmol), stirred for 30 minutes, then treated with sodium cyanoborohydride (10.76 mg, 0.171 mmol). The reaction was stirred for 16 hours. The reaction was diluted with minimal drops of water to bring about dissolution, then the reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 40-100% B over 20 minutes, then a 5-minute hold at 100% B; Flow: 20 mL/min. Fractions containing the desired product were combined and dried via centrifugal evaporation, affording (0.0035 g, 5.85 μmol, 6.84% yield). The estimated purity by LCMS analysis was 99%. LCMS (Condition ACN-AA, ES+) M+H=592.1, 1.94 minutes, calculated exact mass=591.239.

### Intermediate: Methyl 1-(2-(4-chloro-5-((3-(2,3-dihydrobenzo[b][1,4]dioxin-6-yl)-2-methylbenzyl)oxy)-2-formylphenoxy)ethyl)-4-hydroxypiperidine-4-carboxylate

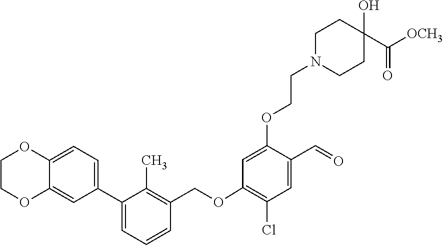 [SMILES: COC(=O)C1(O)CCN(CCOc2cc(OCc3cccc(-c4ccc5c(c4)OCCO5)c3C)c(Cl)cc2C=O)CC1]

A solution of 2-(2-bromoethoxy)-5-chloro-4-((3-(2,3-dihydrobenzo[b][1,4]dioxin-6-yl)-2-methylbenzyl)oxy)benzaldehyde (0.209 g, 0.404 mmol) in dry N,N-dimethylformamide (5.0 mL) was treated with cesium carbonate (0.329 g, 1.009 mmol) followed by methyl 4-hydroxypiperidine-4-carboxylate hydrochloride (0.158 g, 0.807 mmol). The mixture was stirred with heating (70° C. oil bath) for 24 hours. The reaction was diluted with ethyl acetate (20 mL) and washed with water (20 mL) followed by brine (20 mL). The organic layer was dried over sodium sulfate, filtered, and concentrated under reduced pressure. The residue was purified by biotage (RediSep 12 g SiO₂, 0% (3 CV), 0-100% (20 CV), 100% (6 CV), ethyl acetate in hexanes). Product fractions were pooled and concentrated under reduced pressure, affording the product (0.117 g, 0.196 mmol, 48.6% yield) as an off-white glassy solid. LCMS (ES+) M+H=596.286. ¹H NMR (400 MHz, CDCl₃) δ: 10.30 (s, 1H), 7.88 (s, 1H), 7.45 (dd, J=5.9, 3.1 Hz, 1H), 7.27-7.24 (m, 2H), 6.92 (d, J=8.3 Hz, 1H), 6.83 (d, J=2.0 Hz, 1H), 6.78 (dd, J=8.3, 2.0 Hz, 1H), 6.64 (s, 1H), 5.23 (s, 2H), 4.32 (s, 4H), 4.21 (t, J=5.8 Hz, 2H), 3.79 (s, 3H), 2.90 (d, J=5.5 Hz, 2H), 2.82 (d, J=11.3 Hz, 2H), 2.59 (td, J=11.8, 2.5 Hz, 2H), 2.30 (s, 3H), 2.11 (td, J=12.7, 4.6 Hz, 2H), 1.65 (dd, J=13.7, 2.6 Hz, 2H).

### Example 1051: (S)-1-(5-Chloro-4-((3-(2,3-dihydrobenzo[b][1,4]dioxin-6-yl)-2-methylbenzyl)oxy)-2-(2-(4-hydroxy-4-(methoxycarbonyl)piperidin-1-yl)ethoxy)benzyl)piperidine-2-carboxylic acid

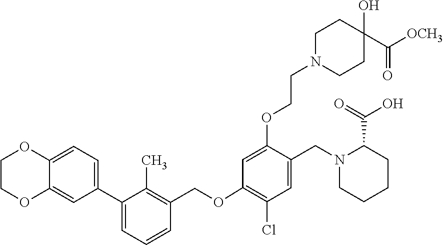 [SMILES: COC(=O)C1(O)CCN(CCOc2cc(OCc3cccc(-c4ccc5c(c4)OCCO5)c3C)c(Cl)cc2CN2CCCC[C@H]2C(=O)O)CC1]

A suspension of methyl 1-(2-(4-chloro-5-((3-(2,3-dihydrobenzo[b][1,4]dioxin-6-yl)-2-methylbenzyl)oxy)-2-formylphenoxy)ethyl)-4-hydroxypiperidine-4-carboxylate (0.037 g, 0.062 mmol) and (S)-piperidine-2-carboxylic acid (0.016 g, 0.124 mmol) in dry N,N-dimethylformamide (0.9 ml) was treated with acetic acid (0.018 ml, 0.310 mmol), stirred for 30 minutes, then treated with sodium cyanoborohydride (7.80 mg, 0.124 mmol). The reaction was stirred for 16 hours. The reaction was diluted with minimal drops of water to bring about dissolution, then the reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 18-62% B over 25 minutes, then a 5-minute hold at 100% B; Flow: 20 mL/min. Fractions containing the desired product were combined and dried via centrifugal evaporation, affording the product (0.0242 g, 0.034 mmol, 54.4% yield). The estimated purity by LCMS analysis was 99%. LCMS (Condition ACN-AA, ES+) M+H=709.3, 1.62 minutes, calculated exact mass=708.281.

### Example 1052: (R)-2-((5-Chloro-4-((3-(2,3-dihydrobenzo[b][1,4]dioxin-6-yl)-2-methylbenzyl)oxy)-2-(2-(4-hydroxy-4-(methoxycarbonyl)piperidin-1-yl)ethoxy)benzyl)amino)-3-hydroxy-2-methylpropanoic acid

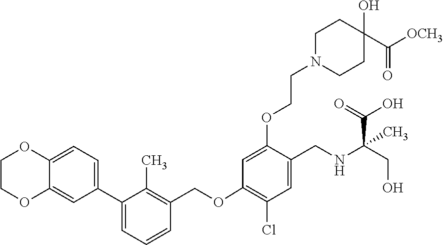 [SMILES: COC(=O)C1(O)CCN(CCOc2cc(OCc3cccc(-c4ccc5c(c4)OCCO5)c3C)c(Cl)cc2CN[C@](C)(CO)C(=O)O)CC1]

A solution of methyl 1-(2-(4-chloro-5-((3-(2,3-dihydrobenzo[b][1,4]dioxin-6-yl)-2-methylbenzyl)oxy)-2-formylphenoxy)ethyl)-4-hydroxypiperidine-4-carboxylate (0.035 g, 0.059 mmol) and (R)-2-amino-3-hydroxy-2-methylpropanoic acid (0.014 g, 0.117 mmol) in dry N,N-dimethylformamide (0.864 ml) was treated with acetic acid (0.017 ml, 0.294 mmol), stirred for 30 minutes, then treated with sodium cyanoborohydride (7.38 mg, 0.117 mmol). The mixture was stirred for 16 hours. The reaction was diluted with several drops of water to dissolve solids, filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation, affording the product (0.032 g, 0.046 mmol, 78% yield). The estimated purity by LCMS analysis was 100%. LCMS (Condition ACN-AA, ES+) M+H=699.1, 1.60 minutes, calculated exact mass=698.261.

### Example 1053: Methyl 1-(2-(4-chloro-5-((3-(2,3-dihydrobenzo[b][1,4]dioxin-6-yl)-2-methylbenzyl)oxy)-2-(((1,3-dihydroxy-2-methylpropan-2-yl)amino)methyl)phenoxy)ethyl)-4-hydroxypiperidine-4-carboxylate

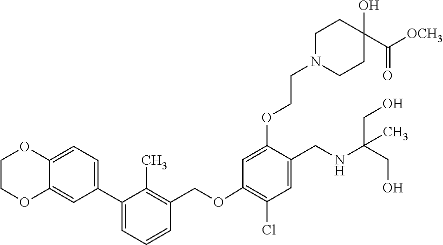 [SMILES: COC(=O)C1(O)CCN(CCOc2cc(OCc3cccc(-c4ccc5c(c4)OCCO5)c3C)c(Cl)cc2CNC(C)(CO)CO)CC1]

A solution of methyl 1-(2-(4-chloro-5-((3-(2,3-dihydrobenzo[b][1,4]dioxin-6-yl)-2-methylbenzyl)oxy)-2-formylphenoxy)ethyl)-4-hydroxypiperidine-4-carboxylate (0.053 g, 0.089 mmol) and 2-amino-2-methylpropane-1,3-diol (0.019 g, 0.178 mmol) in dry N,N-dimethylformamide (1.308 ml) was treated with acetic acid, stirred for 30 minutes, then treated with sodium cyanoborohydride (0.011 g, 0.178 mmol). The mixture was stirred for 16 hours. The reaction was filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-70% B over 25 minutes, then a 5-minute hold at 100% B; Flow: 20 mL/min. Fractions containing the desired product were combined and dried via centrifugal evaporation, affording the product (0.0462 g, 0.067 mmol, 76% yield). The estimated purity by LCMS analysis was 100%. LCMS (Condition ACN-AA, ES+) M+H=685.1, 1.73 minutes, calculated exact mass=684.281. ¹H NMR (500 MHz, DMSO-d₆) δ: 7.47 (d, J=7.3 Hz, 1H), 7.37 (s, 1H), 7.30-7.21 (m, 1H), 7.17 (d, J=7.0 Hz, 1H), 6.97 (s, 1H), 6.92 (d, J=8.1 Hz, 1H), 6.79-6.69 (m, 2H), 5.25 (s, 2H), 4.28 (s, 4H), 4.15 (t, J=5.5 Hz, 2H), 3.69 (br. s., 1H), 3.63 (s, 2H), 2.72 (t, J=5.7 Hz, 2H), 2.62 (d, J=11.4 Hz, 2H), 2.48-2.40 (m, 2H), 2.24 (s, 3H), 1.94-1.81 (m, 5H), 1.60 (d, J=13.9 Hz, 2H), 1.00 (s, 3H); several peaks were solvent obscured.

### Example 1054: (S)-1-(2-(2-(4-Carboxy-4-hydroxypiperidin-1-yl)ethoxy)-5-chloro-4-((3-(2,3-dihydrobenzo[b]1[1,4]dioxin-6-yl)-2-methylbenzyl)oxy)benzyl)piperidine-2-carboxylic acid

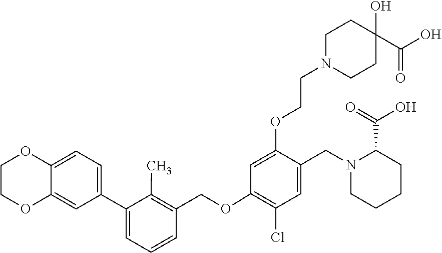 [SMILES: Cc1c(COc2cc(OCCN3CCC(O)(C(=O)O)CC3)c(CN3CCCC[C@H]3C(=O)O)cc2Cl)cccc1-c1ccc2c(c1)OCCO2]

A solution of (S)-1-(5-chloro-4-((3-(2,3-dihydrobenzo[b][1,4]dioxin-6-yl)-2-methylbenzyl)oxy)-2-(2-(4-hydroxy-4-(methoxycarbonyl)piperidin-1-yl)ethoxy)benzyl) piperidine-2-carboxylic acid (0.0239 g, 0.034 mmol) in methanol (0.2 mL) was treated with a solution of lithium hydroxide monohydrate (6.0 mg, 0.143 mmol) in water (0.2 mL), and the mixture was stirred for 6 hours. The reaction was diluted with methanol, filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 23-63% B over 25 minutes, then a 5-minute hold at 100% B; Flow: 20 mL/min. Fractions containing the desired product were combined and dried via centrifugal evaporation, affording the product (0.0149 g, 0.021 mmol, 61.1% yield). The estimated purity by LCMS analysis was 96%. LCMS (Condition ACN-AA, ES+) M+H=695.1, 1.58 minutes, calculated exact mass=694.266.

### Example 1055: (R)-1-(2-(2-(((2-Carboxy-1-hydroxypropan-2-yl)amino)methyl)-4-chloro-5-((3-(2,3-dihydrobenzo[b]1[1,4]dioxin-6-yl)-2-methylbenzyl)oxy)phenoxy)ethyl)-4-hydroxypiperidine-4-carboxylic acid

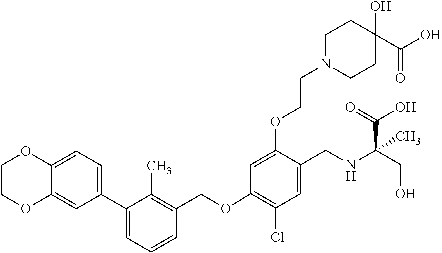 [SMILES: Cc1c(COc2cc(OCCN3CCC(O)(C(=O)O)CC3)c(CN[C@](C)(CO)C(=O)O)cc2Cl)cccc1-c1ccc2c(c1)OCCO2]

A solution of (R)-2-((5-chloro-4-((3-(2,3-dihydrobenzo[b][1,4]dioxin-6-yl)-2-methylbenzyl)oxy)-2-(2-(4-hydroxy-4-(methoxycarbonyl)piperidin-1-yl)ethoxy) benzyl)amino)-3-hydroxy-2-methylpropanoic acid (0.026 g, 0.037 mmol) in methanol (0.2 mL) was treated with a solution of lithium hydroxide monohydrate (6.0 mg, 0.143 mmol) in water (0.2 mL), and the mixture was stirred for 3 hours. The reaction was diluted with methanol, filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation, affording the product (0.0142 g, 0.021 mmol, 55.2% yield). The estimated purity by LCMS analysis was 99%. LCMS (Condition ACN-AA, ES+) M+H=685.1, 1.49 minutes, calculated exact mass=684.254.

### Example 1056: 1-(2-(4-Chloro-5-((3-(2,3-dihydrobenzo[b][1,4]dioxin-6-yl)-2-methylbenzyl)oxy)-2-(((1,3-dihydroxy-2-methylpropan-2-yl)amino)methyl)phenoxy)ethyl)-4-hydroxypiperidine-4-carboxylic acid

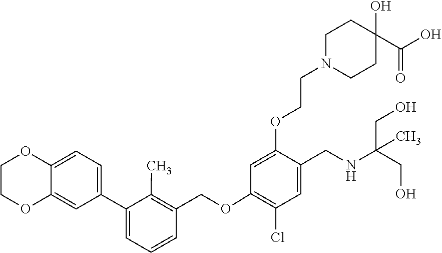 [SMILES: Cc1c(COc2cc(OCCN3CCC(O)(C(=O)O)CC3)c(CNC(C)(CO)CO)cc2Cl)cccc1-c1ccc2c(c1)OCCO2]

A solution of methyl 1-(2-(4-chloro-5-((3-(2,3-dihydrobenzo[b][1,4]dioxin-6-yl)-2-methylbenzyl)oxy)-2-(((1,3-dihydroxy-2-methylpropan-2-yl)amino)methyl)phenoxy) ethyl)-4-hydroxypiperidine-4-carboxylate (0.0364 g, 0.053 mmol) in methanol (0.2 mL) was treated with a solution of lithium hydroxide monohydrate (8.0 mg, 0.191 mmol) in water (0.2 mL), and the mixture was stirred for 3 hours. The reaction was diluted with methanol, filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation, affording (0.019 g, 0.028 mmol, 53.3% yield). The estimated purity by LCMS analysis was 100%. LCMS (Condition ACN-AA, ES+) M+H=671.1, 1.55 minutes, calculated exact mass=670.266. Poor quality 1H NMR spectrum obtained.

### Intermediate: 5-Chloro-4-((3-(2,3-dihydrobenzo[b][1,4]dioxin-6-yl)-2-methylbenzyl) oxy)-2-(2-(3-hydroxy-3-(trifluoromethyl)piperidin-1-yl)ethoxy)benzaldehyde

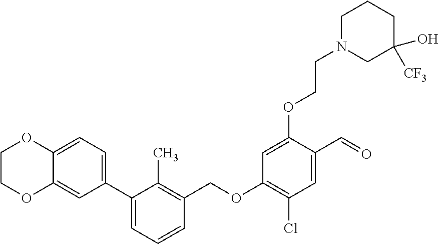 [SMILES: Cc1c(COc2cc(OCCN3CCCC(O)(C(F)(F)F)C3)c(C=O)cc2Cl)cccc1-c1ccc2c(c1)OCCO2]

A solution of 2-(2-bromoethoxy)-5-chloro-4-((3-(2,3-dihydrobenzo[b][1,4]dioxin-6-yl)-2-methylbenzyl)oxy)benzaldehyde (0.120 g, 0.232 mmol) in dry N,N-dimethylformamide (2.318 ml) was treated with cesium carbonate (0.202 g, 0.620 mmol) followed by 3-(trifluoromethyl)piperidin-3-ol hydrochloride (0.113 g, 0.550 mmol). The mixture was stirred with heating (70° C. oil bath) for 6 hours. Additional 3-(trifluoromethyl)piperidin-3-ol hydrochloride (0.113 g, 0.550 mmol) was added and the reaction was stirred for 16 hours. The reaction was treated with additional 3-(trifluoromethyl)piperidin-3-ol hydrochloride (0.113 g, 0.550 mmol) and cesium carbonate (0.202 g, 0.620 mmol), and heated for 6 hours then cooled. The reaction was diluted with ethyl acetate (20 mL) and washed with water (20 mL) followed by brine (20 mL). The organic layer was dried over sodium sulfate, filtered, and concentrated under reduced pressure. Extended drying (10 days) of sample under vacuume afforded the product as a brown viscous oil. LCMS (ES+) M+H=606.1, 0.89 minutes, calculated exact mass=605.179. The product was used as is in following reactions.

### Example 1057: (2R)-2-((5-Chloro-4-((3-(2,3-dihydrobenzo[b][1,4]dioxin-6-yl)-2-methylbenzyl)oxy)-2-(2-(3-hydroxy-3-(trifluoromethyl)piperidin-1-yl)ethoxy)benzyl)amino)-3-hydroxy-2-methylpropanoic acid

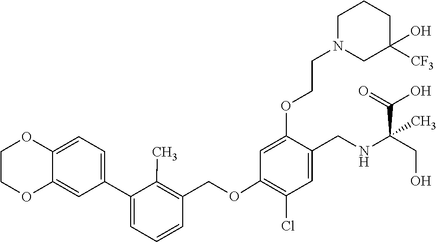 [SMILES: Cc1c(COc2cc(OCCN3CCCC(O)(C(F)(F)F)C3)c(CN[C@](C)(CO)C(=O)O)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-(2-(3-hydroxy-3-(trifluoromethyl)piperidin-1-yl)ethoxy) benzaldehyde (0.035 g, 0.058 mmol) and (R)-2-amino-3-hydroxy-2-methylpropanoic acid (0.014 g, 0.116 mmol) in dry N,N-dimethylformamide (1.7 mL) was treated with acetic acid (0.017 mL, 0.289 mmol), stirred for 30 minutes, then treated with sodium cyanoborohydride (7.26 mg, 0.116 mmol). The reaction was stirred for 19 hours. The reaction was diluted with several drops of water, then filtered (0.45 μm syringe-tip filter) and purified by preparative LC/MS with the following conditions: Mobile Phase A: 5:95 acetonitrile: water with 10-mM ammonium acetate; Mobile Phase B: 95:5 acetonitrile: water with 10-mM ammonium acetate; Gradient: 40-100% B over 25 minutes, then a 5-minute hold at 100% B; Flow: 20 mL/min. Fractions containing the desired product were combined and dried via centrifugal evaporation, affording the product (0.0124 g, 0.017 mmol, 30.3% yield). The estimated purity by LCMS analysis was 100%. LCMS (Condition ACN-AA, ES+) M+H=709.1, 1.99 minutes, calculated exact mass=708.243. ¹H NMR (500 MHz, DMSO-d₆) δ: 7.49 (d, J=7.7 Hz, 1H), 7.45 (s, 1H), 7.26 (t, J=7.5 Hz, 1H), 7.18 (d, J=7.0 Hz, 1H), 7.02 (d, J=4.0 Hz, 1H), 6.92 (d, J=8.1 Hz, 1H), 6.79-6.71 (m, 2H), 5.28 (s, 2H), 4.28 (s, 4H), 4.25-4.10 (m, 2H), 3.94-3.81 (m, 2H), 3.66-3.47 (m, 1H), 2.88-2.75 (m, 3H), 2.29 (t, J=12.8 Hz, 1H), 2.24 (s, 3H), 2.15-2.00 (m, 1H), 1.90 (s, 2H), 1.84-1.72 (m, 1H), 1.64 (d, J=12.1 Hz, 1H), 1.56-1.37 (m, 2H), 1.25 (d, J=4.0 Hz, 3H).

### Example 1058: (2S)-1-(5-Chloro-4-((3-(2,3-dihydrobenzo[b][1,4]dioxin-6-yl)-2-methylbenzyl)oxy)-2-(2-(3-hydroxy-3-(trifluoromethyl)piperidin-1-yl)ethoxy)benzyl)piperidine-2-carboxylic acid

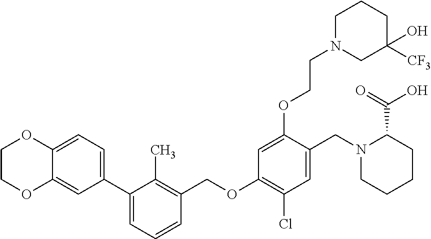 [SMILES: Cc1c(COc2cc(OCCN3CCCC(O)(C(F)(F)F)C3)c(CN3CCCC[C@H]3C(=O)O)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-(2-(3-hydroxy-3-(trifluoromethyl)piperidin-1-yl)ethoxy)benzaldehyde (0.035 g, 0.058 mmol) and (S)-piperidine-2-carboxylic acid (0.015 g, 0.116 mmol) in dry N,N-dimethylformamide (1.7 mL) was treated with acetic acid (0.017 mL, 0.289 mmol), stirred for 30 minutes, then treated with sodium cyanoborohydride (7.26 mg, 0.116 mmol). The reaction was stirred for 19 hours. The reaction was diluted with several drops of water, then filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 40-100% B over 25 minutes, then a 5-minute hold at 100% B; Flow: 20 mL/min. Fractions containing the desired product were combined and dried via centrifugal evaporation, affording the product (0.0153 g, 0.020 mmol, 33.9% yield). The estimated purity by LCMS analysis was 92%. LCMS (ACN-AA, ES+) M+H=719.1, 2.06 minutes, calculated exact mass=718.263.

### Example 1059: 2-((5-Chloro-4-((3-(2,3-dihydrobenzo[b][1,4]dioxin-6-yl)-2-methylbenzyl)oxy)-2-(2-(3-hydroxy-3-(trifluoromethyl)piperidin-1-yl)ethoxy)benzyl)amino)-2-methylpropane-1,3-diol

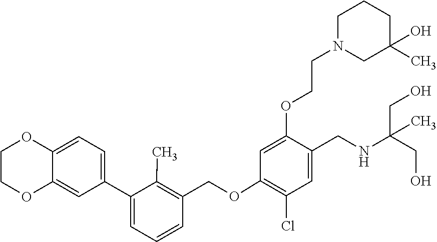 [SMILES: Cc1c(COc2cc(OCCN3CCCC(C)(O)C3)c(CNC(C)(CO)CO)cc2Cl)cccc1-c1ccc2c(c1)OCCO2]

A solution of 5-chloro-4-((3-(2,3-dihydrobenzo[b][1,4]dioxin-6-yl)-2-methylbenzyl)oxy)-2-(2-(3-hydroxy-3-(trifluoromethyl)piperidin-1-yl)ethoxy) benzaldehyde (0.035 g, 0.058 mmol) and (R)-2-amino-3-hydroxy-2-methylpropanoic acid (0.014 g, 0.116 mmol) in dry N,N-dimethylformamide (1.7 mL) was treated with acetic acid (0.017 mL, 0.289 mmol), stirred for 30 minutes, then treated with sodium cyanoborohydride (7.26 mg, 0.116 mmol). The reaction was stirred for 19 hours. The reaction was diluted with several drops of water, then filtered (0.45 μm syringe-tip filter) and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 45-100% B over 25 minutes, then a 5-minute hold at 100% B; Flow: 20 mL/min. Fractions containing the desired product were combined and dried via centrifugal evaporation, affording the product (0.0105 g, 0.015 mmol, 26.2% yield). The estimated purity by LCMS analysis was 100%. LCMS (Condition ACN-AA, ES+) M+H=695.1, 2.07 minutes, calculated exact mass=694.263. ¹H NMR (500 MHz, DMSO-d₆) δ: 7.48 (d, J=7.6 Hz, 1H), 7.36 (s, 1H), 7.29-7.21 (m, 1H), 7.17 (d, J=7.0 Hz, 1H), 6.97-6.89 (m, 2H), 6.79-6.70 (m, 2H), 5.24 (s, 2H), 4.28 (s, 4H), 4.22-4.10 (m, 2H), 2.87 (d, J=11.0 Hz, 2H), 2.81 (t, J=5.5 Hz, 2H), 2.34 (d, J=11.6 Hz, 1H), 2.24 (s, 3H), 2.19-2.08 (m, 1H), 1.86 (s, 5H), 1.82-1.72 (m, 1H), 1.66 (d, J=12.8 Hz, 1H), 1.57-1.40 (m, 2H), 0.93 (s, 2H).

### Example 1060: (S)-1-(5-chloro-4-((3-(2,3-dihydrobenzo[b][1,4]dioxin-6-yl)-2-methylbenzyl)oxy)-2-(2-(1,1-dioxidothiomorpholino)ethoxy)benzyl)piperidine-2-carboxylic acid

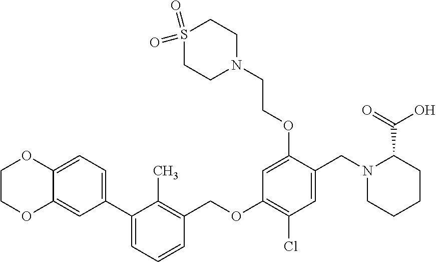 [SMILES: Cc1c(COc2cc(OCCN3CCS(=O)(=O)CC3)c(CN3CCCC[C@H]3C(=O)O)cc2Cl)cccc1-c1ccc2c(c1)OCCO2]

Example 1060 was prepared in a similar manner as described above.

### Example 1061: (S)-1-(5-chloro-4-((3-(2,3-dihydrobenzo[b][1,4]dioxin-6-yl)-2-methylbenzyl)oxy)-2-(2-(1,1-dioxidothiomorpholino)-2-oxoethoxy)benzyl)piperidine-2-carboxylic acid

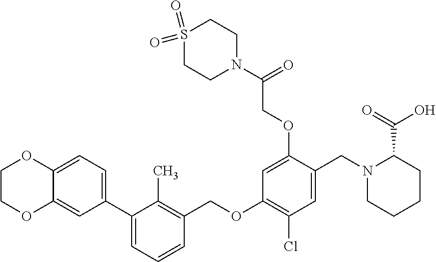 [SMILES: Cc1c(COc2cc(OCC(=O)N3CCS(=O)(=O)CC3)c(CN3CCCC[C@H]3C(=O)O)cc2Cl)cccc1-c1ccc2c(c1)OCCO2]

Example 1061 was prepared in a similar manner as described above.

### Intermediate: 5-chloro-4-((3-(2,3-dihydrobenzo[b][1,4]dioxin-6-yl)-2-methylbenzyl)oxy)-2-(3-(dimethylamino)propoxy)benzaldehyde

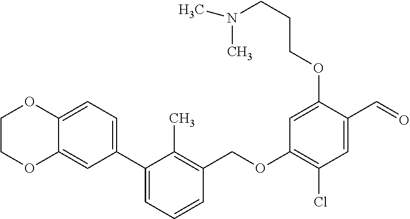 [SMILES: Cc1c(COc2cc(OCCCN(C)C)c(C=O)cc2Cl)cccc1-c1ccc2c(c1)OCCO2]

The mixture of 5-chloro-4-((3-(2,3-dihydrobenzo[b][1,4]dioxin-6-yl)-2-methylbenzyl)oxy)-2-hydroxybenzaldehyde (60 mg, 0.146 mmol), 3-chloro-1-(N,N-dimethyl)propylamine (19.54 mg, 0.161 mmol), and cesium carbonate (95 mg, 0.292 mmol) in DMF (2 mL) was stirred at 75° C. for 3 hrs. The solvent was removed from the reaction and the residue was partitioned between dichloromethane and water. The aqueous phase was extracted once with dichloromethane. The organic extracts were combined and washed with brine and then dried over sodium sulfate. The drying agent was removed, and the solvent was removed to give 110 mg of the crude product, which was directly for the next step without further purification.

### Example 2001: (S)-2-((5-chloro-4-((3-(2,3-dihydrobenzo[b][1,4]dioxin-6-yl)-2-methylbenzyl)oxy)-2-(3-(dimethylamino)propoxy)benzyl)amino)-3-hydroxy-2-methylpropanoic acid

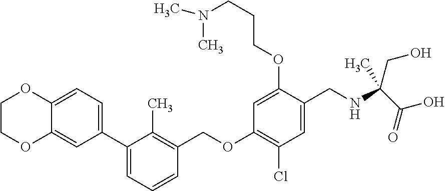 [SMILES: Cc1c(COc2cc(OCCCN(C)C)c(CN[C@@](C)(CO)C(=O)O)cc2Cl)cccc1-c1ccc2c(c1)OCCO2]

(S)-2-((5-Chloro-4-((3-(2,3-dihydrobenzo[b][1,4]dioxin-6-yl)-2-methylbenzyl) oxy)-2-(3-(dimethylamino)propoxy)benzyl)amino)-3-hydroxy-2-methylpropanoic acid was obtained from 5-chloro-4-((3-(2,3-dihydrobenzo[b][1,4]dioxin-6-yl)-2-methylbenzyl) oxy)-2-(3-(dimethylamino)propoxy)benzaldehyde and 2-methyl-L-serine by using and acetic acid and borane-2-picoline complex. The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 15 minutes, then a 5-minute hold at 100% B; Flow: 20 mL/min. Fractions containing the desired product were combined and dried via centrifugal evaporation. The material was further purified via preparative LC/MS with the following conditions: Column: Waters CSH C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 30-70% B over 15 minutes, then a 5-minute hold at 100% B; Flow: 20 mL/min. Fractions containing the desired product were combined and dried via centrifugal evaporation. ¹H NMR (500 MHz, DMSO-d₆) δ 7.53-7.46 (m, 2H), 7.27 (t, J=7.6 Hz, 1H), 7.19 (d, J=7.3 Hz, 1H), 7.01 (s, 1H), 6.93 (d, J=8.2 Hz, 1H), 6.78-6.71 (m, 2H), 5.29 (s, 2H), 4.27 (s, 4H), 4.15 (br. s., 2H), 4.12-4.04 (m, 2H), 3.79 (d, J=11.9 Hz, 1H), 3.75-3.62 (m, 1H) 3.33-3.26 (m, 2H), 2.81 (s, 6H), 2.51 (br. s., 2H), 2.23 (s, 3H), 1.39 (s, 3H).

Two analytical LC/MS injections were used to determine the final purity.

Injection 1 conditions: Column: Waters BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 70° C.; Gradient: 0% B, 0-100% B over 3 minutes, then a 2.0-minute hold at 100% B; Flow: 0.75 mL/min; Detection: UV at 220 nm.

Injection 2 conditions: Column: Waters CSH C18, 2.1×50 mm, 1.7-μm particles; Mobile Phase A: 5:95 acetonitrile:water with trifluoroacetic acid; Mobile Phase B: 95:5 acetonitrile:water with trifluoroacetic acid; Temperature: 70° C.; Gradient: 0% B, 0-100% B over 3 minutes, then a 2.0-minute hold at 100% B; Flow: 0.75 mL/min; Detection: UV at 220 nm.

LCMS (Injection 1 condition) Rt=1.58 min. m/z 600 (M+1).

LCMS (Injection 2 condition) Rt=1.429 min, m/z 600 (M+1).

### Example 2002: (S)-2-((5-chloro-4-((3-(2,3-dihydrobenzo[b][1,4]dioxin-6-yl)-2-methylbenzyl)oxy)-2-(3-(dimethylamino)propoxy)benzyl)amino)-3-hydroxypropanoic acid

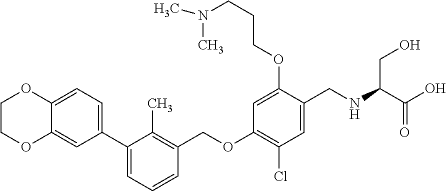 [SMILES: Cc1c(COc2cc(OCCCN(C)C)c(CN[C@@H](CO)C(=O)O)cc2Cl)cccc1-c1ccc2c(c1)OCCO2]

(S)-2-((5-Chloro-4-((3-(2,3-dihydrobenzo[b][1,4]dioxin-6-yl)-2-methylbenzyl) oxy)-2-(3-(dimethylamino)propoxy)benzyl)amino)-3-hydroxypropanoic acid was obtained from 5-chloro-4-((3-(2,3-dihydrobenzo[b][1,4]dioxin-6-yl)-2-methylbenzyl) oxy)-2-(3-(dimethylamino)propoxy)benzaldehyde and L-serine by using similar conditions for Example 2001. The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 40 minutes, then a 5-minute hold at 100% B; Flow: 20 m/min. Fractions containing the desired product were combined and dried via centrifugal evaporation. ¹H NMR (500 MHz, DMSO-d₆) δ 7.49 (d, J=7.7 Hz, 1H), 7.44 (s, 1H), 7.30-7.23 (m, 1H), 7.18 (d, J=7.3 Hz, 1H), 6.98 (s, 1H), 6.93 (d, J=8.1 Hz, 1H), 6.79-6.72 (m, 2H), 5.27 (s, 2H), 4.28 (s, 4H), 4.11 (t, J=6.2 Hz, 2H), 3.91 (d, J=5.1 Hz, 2H), 3.69-3.63 (m, 1H), 3.61 (d, J=6.6 Hz, 1H), 3.08 (t, J=5.1 Hz, 1H), 2.53-2.45 (m, 2H), 2.27-2.19 (m, 9H), 1.94 (t, J=6.6 Hz, 2H).

Two analytical LC/MS injections were used to determine the final purity.

Injection 1 conditions: Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm.

Injection 2 conditions: Column: Waters BEH C18, 2.0×50 mm, 1.7-μm particles; Mobile Phase A: 5:95 methanol:water with 10 mM ammonium acetate; Mobile Phase B: 95:5 methanol:water with 10 mM ammonium acetate; Temperature: 50° C.; Gradient: 0% B, 0-100% B over 3 minutes, then a 0.5-minute hold at 100% B; Flow: 0.5 mL/min; Detection: UV at 220 nm.

LCMS (Injection 1 condition) Rt=1.47 min, m/z 585 (M+1), 583 (M-1).

LCMS (Injection 2 condition) Rt=2.53 min, m/z 585 (M+1), 583 (M-1).

### Intermediate: 5-chloro-4-((3-(2,3-dihydrobenzo[b][1,4]dioxin-6-yl)-2-methylbenzyl)oxy)-2-((1-methylpiperidin-3-yl)methoxy)benzaldehyde

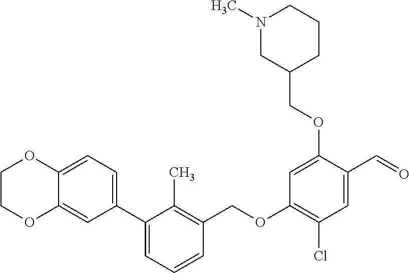 [SMILES: Cc1c(COc2cc(OCC3CCCN(C)C3)c(C=O)cc2Cl)cccc1-c1ccc2c(c1)OCCO2]

5-Chloro-4-((3-(2,3-dihydrobenzo[b][1,4]dioxin-6-yl)-2-methylbenzyl)oxy)-2-((1-methylpiperidin-3-yl)methoxy)benzaldehyde (crude) was obtained from 5-chloro-4-((3-(2,3-dihydrobenzo[b][1,4]dioxin-6-yl)-2-methylbenzyl)oxy)-2-hydroxybenzaldehyde and 3-chloromethyl-1-methylpiperidine hydrochloride using the procedure described for 5-chloro-4-((3-(2,3-dihydrobenzo[b][1,4]dioxin-6-yl)-2-methylbenzyl)oxy)-2-(3-(dimethylamino)propoxy)benzaldehyde.

### Example 2003: (2S)-2-((5-chloro-4-((3-(2,3-dihydrobenzo[b][1,4]dioxin-6-yl)-2-methylbenzyl)oxy)-2-((1-methylpiperidin-3-yl)methoxy)benzyl)amino)-3-hydroxy-2-methylpropanoic acid

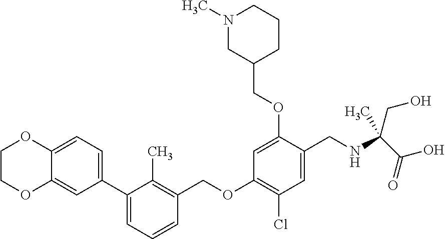 [SMILES: Cc1c(COc2cc(OCC3CCCN(C)C3)c(CN[C@@](C)(CO)C(=O)O)cc2Cl)cccc1-c1ccc2c(c1)OCCO2]

(2S)-2-((5-Chloro-4-((3-(2,3-dihydrobenzo[b][1,4]dioxin-6-yl)-2-methylbenzyl)oxy)-2-((1-methylpiperidin-3-yl)methoxy)benzyl)amino)-3-hydroxy-2-methylpropanoic acid was obtained from 5-chloro-4-((3-(2,3-dihydrobenzo[b][1,4]dioxin-6-yl)-2-methylbenzyl)oxy)-2-((1-methylpiperidin-3-yl)methoxy)benzaldehyde and 2-methyl-L-serine by using similar conditions for Example 2001. The crude material was purified via preparative LC/MS with the following conditions: Column: XBridge C18, 19×200 mm, 5-μm particles; Mobile Phase A: 5:95 methanol: water with 10-mM ammonium acetate; Mobile Phase B: 95:5 methanol: water with 10-mM ammonium acetate; Gradient: 50-100% B over 20 minutes, then a 5-minute hold at 100% B; Flow: 20 mL/min. Fractions containing the desired product were combined and dried via centrifugal evaporation. ¹H NMR (500 MHz, DMSO-d₆) δ 7.51-7.44 (m, 2H), 7.26 (t, J=7.5 Hz, 1H), 7.18 (d, J=7.7 Hz, 1H), 6.97 (s, 1H), 6.92 (d, J=8.1 Hz, 1H), 6.79-6.72 (m, 2H), 5.26 (s, 2H), 4.28 (s, 4H), 4.03-3.87 (m, 4H), 3.66 (d, J=11.4 Hz, 1H), 3.54 (d, J=11.4 Hz, 1H), 2.99 (br. s., 1H), 2.73 (br, s, 1H), 2.27 (d, J=12.1 Hz, 6H), 2.18-1.99 (m, 3H), 1.79-1.49 (m, 3H), 1.28 (s, 3H), 1.16 (br, s, 1H).

Two analytical LC/MS injections were used to determine the final purity.

Injection 1 conditions: Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm.

Injection 2 conditions: Column: Waters BEH C18, 2.0×50 mm, 1.7-μm particles; Mobile Phase A: 5:95 methanol:water with 10 mM ammonium acetate; Mobile Phase B: 95:5 methanol:water with 10 mM ammonium acetate; Temperature: 50° C.; Gradient: 0% B, 0-100% B over 3 minutes, then a 0.5-minute hold at 100% B; Flow: 0.5 mL/min; Detection: UV at 220 nm.

LCMS (Injection 1 condition) Rt=1.55 min, m/z 625 (M+1), 623 (M-1).

LCMS (Injection 2 condition) Rt=2.60 min, m/z 625 (M+1). 623 (M-1).

### Example 2004: (2S)-2-((5-chloro-4-((3-(2,3-dihydrobenzo[b][1,4]dioxin-6-yl)-2-methylbenzyl)oxy)-2-((1-methylpiperidin-3-yl)methoxy)benzyl)amino)-3-hydroxypropanoic acid

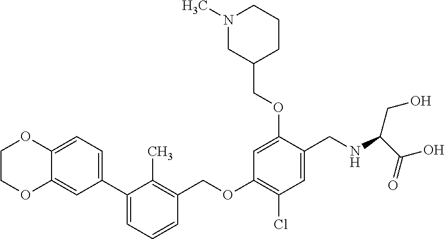 [SMILES: Cc1c(COc2cc(OCC3CCCN(C)C3)c(CN[C@@H](CO)C(=O)O)cc2Cl)cccc1-c1ccc2c(c1)OCCO2]

(2S)-2-((5-Chloro-4-((3-(2,3-dihydrobenzo[b][1,4]dioxin-6-yl)-2-methylbenzyl) oxy)-2-((1-methylpiperidin-3-yl)methoxy)benzyl)amino)-3-hydroxypropanoic acid was obtained from 5-chloro-4-((3-(2,3-dihydrobenzo[b][1,4]dioxin-6-yl)-2-methylbenzyl) oxy)-2-((1-methylpiperidin-3-yl)methoxy)benzaldehyde and L-serine by using similar conditions for Example 2001. The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 15 minutes, then a 5-minute hold at 100% B; Flow: 20 mL/min. Fractions containing the desired product were combined and dried via centrifugal evaporation. ¹H NMR (500 MHz, DMSO-d₆) δ 7.49-7.43 (m, 2H), 7.28-7.21 (m, 1H), 7.17 (d, J=7.3 Hz, 1H), 6.96 (s, 1H), 6.91 (d, J=8.1 Hz, 1H), 6.78-6.71 (m, 2H), 5.24 (s, 2H), 4.27 (s, 4H), 4.07-3.85 (m, 4H), 3.76-3.69 (m, 1H), 3.68-3.61 (m, 1H), 3.18-3.13 (m, 1H), 2.99 (t, J=11.0 Hz, 1H), 2.75 (br. s., 1H), 2.28 (s, 3H), 2.23 (s, 3H), 2.19-2.04 (m, 3H), 1.76-1.63 (m, 2H), 1.56 (m, 1H), 1.15 (m, 1H).

Two analytical LC/MS injections were used to determine the final purity.

Injection 1 conditions: Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm.

Injection 2 conditions: Column: Waters BEH C18, 2.0×50 mm, 1.7-μm particles; Mobile

Phase A: 5:95 methanol:water with 10 mM ammonium acetate; Mobile Phase B: 95:5 methanol:water with 10 mM ammonium acetate; Temperature: 50° C.; Gradient: 0% B, 0-100% B over 3 minutes, then a 0.5-minute hold at 100% B; Flow: 0.5 mL/min; Detection: UV at 220 nm.

LCMS (Injection 1 condition) Rt=1.52 min, m/z 611 (M+1), 609 (M-1).

LCMS (Injection 2 condition) Rt=2.56 min, M/z 611 (M+1), 609 (M-1).

### Example 2005 (Isomer-1) and Example 2006 (Isomer-2)

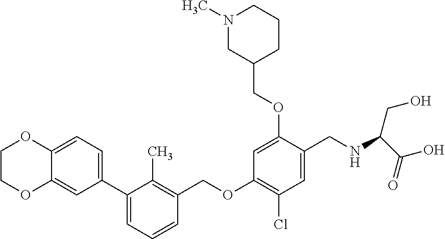 [SMILES: Cc1c(COc2cc(OCC3CCCN(C)C3)c(CN[C@@H](CO)C(=O)O)cc2Cl)cccc1-c1ccc2c(c1)OCCO2]

(2S)-2-((5-Chloro-4-((3-(2,3-dihydrobenzo[b][1,4]dioxin-6-yl)-2-methylbenzyl) oxy)-2-((1-methylpiperidin-3-yl)methoxy)benzyl)amino)-3-hydroxypropanoic acid was further separated by chiral SFC to give two diastereoisomers via the conditions: Column ChiralCel OD-H, 30×250 mm, 5 μm; Mobile Phase:20% zMeOH:H₂O (95:5) w/10 mM NH₄OAc/80% CO₂; Pressure: 100 bar; Flow Rate:70 mL/min; UV:210 nm.

Example 2005 (Isomer-1): Rt=14.374 min. ee=100%, ¹H NMR (400 MHz, METHANOL-d4) δ 7.52-7.43 (m, 2H), 7.28-7.17 (m, 2H), 6.95-6.87 (m, 2H), 6.80-6.70 (m, 2H), 5.29 (s, 2H), 4.30 (s, 4H), 4.22-4.09 (m, 1H), 4.06-3.83 (m, 2H), 3.66-3.48 (m, 1H), 2.75 (br. s., 3H), 2.66 (d, J=11.5 Hz, 1H), 2.29 (s, 3H), 2.06-1.78 (m, 6H), 1.31 (br. s., 4H), 0.91 (d, J=8.8 Hz, 1H).

Example 2006 (Isomer-2): Rt=18.93 min. ee=89.00%, ¹H NMR (400 MHz, METHANOL-d4) δ 7.46 (d, J=5.3 Hz, 2H), 7.30-7.17 (m, 2H), 6.89 (d, J=8.0 Hz, 2H), 6.81-6.71 (m, 2H), 5.30 (br. s., 2H), 4.30 (s, 4H), 4.22-3.89 (m, 2H), 3.74-3.47 (m, 2H), 2.75 (br. s., 4H), 2.29 (s, 3H), 2.10-1.74 (m, 6H), 1.31 (br. s., 4H), 0.99-0.84 (m, 1H).

### Intermediate: (S)-ethyl 5-((4-chloro-5-((3-(2,3-dihydrobenzo[b][1,4]dioxin-6-yl)-2-methylbenzyl)oxy)-2-((3-hydroxypyrrolidin-1-yl)methyl)phenoxy)methyl)nicotinate

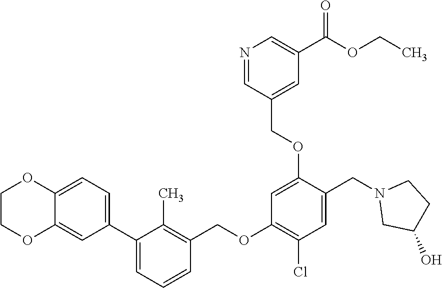 [SMILES: CCOC(=O)c1cncc(COc2cc(OCc3cccc(-c4ccc5c(c4)OCCO5)c3C)c(Cl)cc2CN2CC[C@H](O)C2)c1]

The mixture of ethyl 5-((4-chloro-5-((3-(2,3-dihydrobenzo[b][1,4]dioxin-6-yl)-2-methylbenzyl)oxy)-2-formylphenoxy)methyl)nicotinate (110 mg, 0.192 mmol), (S)-pyrrolidin-3-ol·HCl salt (71.0 mg, 0.575 mmol) and sodium triacetoxyhydroborate (126 mg, 0.594 mmol) in DMF (4 mL) was stirred at room temperature overnight. The solvent was removed. The residue was used in the next step without further purification.

### Intermediate: (S)-5-((4-chloro-5-((3-(2,3-dihydrobenzo[b][1,4]dioxin-6-yl)-2-methylbenzyl)oxy)-2-((3-hydroxypyrrolidin-1-yl)methyl)phenoxy)methyl)nicotinic acid

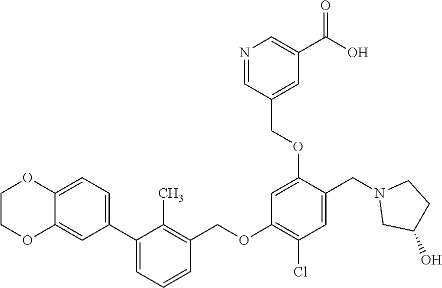 [SMILES: Cc1c(COc2cc(OCc3cncc(C(=O)O)c3)c(CN3CC[C@H](O)C3)cc2Cl)cccc1-c1ccc2c(c1)OCCO2]

Lithium hydroxide (0.064 g, 2.69 mmol) was added to a solution of (S)-ethyl 5-((4-chloro-5-((3-(2,3-dihydrobenzo[b][1,4]dioxin-6-yl)-2-methylbenzyl)oxy)-2-((3-hydroxypyrrolidin-1-yl)methyl)phenoxy)methyl)nicotinate (0.124 g, 0.192 mmol) in THF (5 mL) and EtOH (5 mL). The mixture was heated under microwave at 100° C. for 55 min. The solvent was removed, and the crude compound was used for the next reaction without further purification.

### Example 2007: (S)-5-((4-chloro-5-((3-(2,3-dihydrobenzo[b][1,4]dioxin-6-yl)-2-methylbenzyl)oxy)-2-((3-hydroxypyrrolidin-1-yl)methyl)phenoxy)methyl)-N-(3-(dimethylamino)propyl)nicotinamide

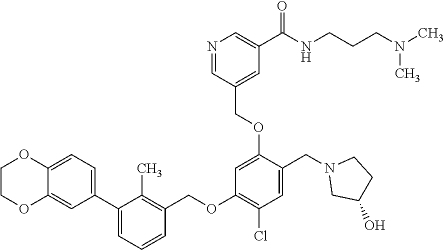 [SMILES: Cc1c(COc2cc(OCc3cncc(C(=O)NCCCN(C)C)c3)c(CN3CC[C@H](O)C3)cc2Cl)cccc1-c1ccc2c(c1)OCCO2]

To a vial was added (S)-5-((4-chloro-5-((3-(2,3-dihydrobenzo[b][1,4]dioxin-6-yl)-2-methylbenzyl)oxy)-2-((3-hydroxypyrrolidin-1-yl)methyl)phenoxy)methyl)nicotinic acid (29.6 mg, 0.048 mmol, crude) in DMF (1 mL) along with N,N-diisopropylethyl amine (0.050 mL, 0.288 mmol) and HATU (54.8 mg, 0.144 mmol). The vial was capped and the mixture stirred at rt for 18 hours. The solvent was removed. The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 5-45% B over 30 minutes, then a 5-minute hold at 100% B; Flow: 20 mL/min. Fractions containing the desired product were combined and dried via centrifugal evaporation to give the target compound. ¹H NMR (400 MHz, METHANOL-d4) δ 8.97 (d, J=2.0 Hz, 1H), 8.84 (d, J=2.0 Hz, 1H), 8.42-8.37 (m, 1H), 7.45-7.40 (m, 2H), 7.25-7.16 (m, 2H), 6.99-6.95 (m, 1H), 6.89 (d, J=8.1 Hz, 1H), 6.78-6.71 (m, 2H), 5.32 (s, 2H), 5.25 (s, 2H), 4.30 (s, 4H), 3.79 (s, 2H), 3.47 (t, J=7.0 Hz, 2H), 2.98-2.84 (m, 2H), 2.72 (td, J=8.9, 5.3 Hz, 1H), 2.64 (dd, J=10.6, 3.1 Hz, 1H), 2.60-2.53 (m, 2H), 2.39 (s, 6H), 2.28 (s, 3H), 2.24-2.11 (m, 1H), 1.96-1.83 (m, 3H), 1.81-1.70 (m, 1H).

Two analytical LC/MS injections were used to determine the final purity.

Injection 1 conditions: Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm.

Injection 2 conditions: Column: Waters BEH C18, 2.0×50 mm, 1.7-μm particles; Mobile Phase A: 5:95 methanol:water with 10 mM ammonium acetate; Mobile Phase B: 95:5 methanol:water with 10 mM ammonium acetate; Temperature: 50° C.; Gradient: 0% B, 0-100% B over 3 minutes, then a 0.5-minute hold at 100% B; Flow: 0.5 mL/min; Detection: UV at 220 nm.

LCMS (Injection 1 condition) Rt=1.46 min, m/z 701(M+1).

LCMS (Injection 2 condition) Rt=3.203 min, m/z 701 (M+1).

### Intermediate: (S)-tert-butyl 3-(5-((4-chloro-5-((3-(2,3-dihydrobenzo[b][1,4]dioxin-6-yl)-2-methylbenzyl)oxy)-2-((3-hydroxypyrrolidin-1-yl)methyl)phenoxy)methyl)nicotinamido)propanoate

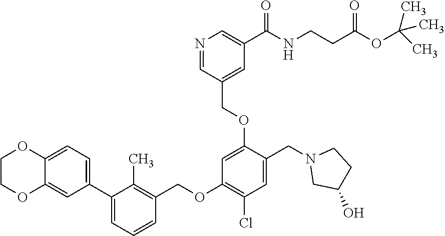 [SMILES: Cc1c(COc2cc(OCc3cncc(C(=O)NCCC(=O)OC(C)(C)C)c3)c(CN3CC[C@H](O)C3)cc2Cl)cccc1-c1ccc2c(c1)OCCO2]

(S)-tert-Butyl 3-(5-((4-chloro-5-((3-(2,3-dihydrobenzo[b][1,4]dioxin-6-yl)-2-methylbenzyl)oxy)-2-((3-hydroxypyrrolidin-1-yl)methyl)phenoxy)methyl) nicotinamido)propanoate (crude) was obtained from (S)-5-((4-chloro-5-((3-(2,3-dihydrobenzo[b][1,4]dioxin-6-yl)-2-methylbenzyl)oxy)-2-((3-hydroxypyrrolidin-1-yl)methyl)phenoxy)methyl)nicotinic acid and H-beta-Ala-OtBu·HCl using the procedure described for Example 2007.

### Intermediate: (S)-ethyl 2-(5-((4-chloro-5-((3-(2,3-dihydrobenzo[b][1,4]dioxin-6-yl)-2-methylbenzyl)oxy)-2-(((S)-3-hydroxypyrrolidin-1-yl)methyl)phenoxy)methyl)nicotinamido)-3-hydroxypropanoate

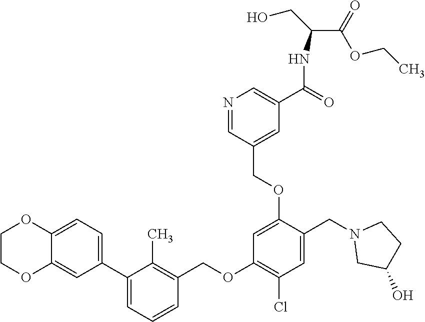 [SMILES: CCOC(=O)[C@H](CO)NC(=O)c1cncc(COc2cc(OCc3cccc(-c4ccc5c(c4)OCCO5)c3C)c(Cl)cc2CN2CC[C@H](O)C2)c1]

(S)-Ethyl 2-(5-((4-chloro-5-((3-(2,3-dihydrobenzo[b][1,4]dioxin-6-yl)-2-methylbenzyl)oxy)-2-(((S)-3-hydroxypyrrolidin-1-yl)methyl)phenoxy)methyl) nicotinamido)-3-hydroxypropanoate (crude) was obtained from (S)-5-((4-chloro-5-((3-(2,3-dihydrobenzo[b]1[1,4]dioxin-6-yl)-2-methylbenzyl)oxy)-2-((3-hydroxypyrrolidin-1-yl)methyl)phenoxy)methyl)nicotinic acid and L-serine ethyl ester hydrochloride using the procedure described for Example 2007.

### Intermediate: (S)-tert-butyl 2-(5-((4-chloro-5-((3-(2,3-dihydrobenzo[b][1,4]dioxin-6-yl)-2-methylbenzyl)oxy)-2-(((S)-3-hydroxypyrrolidin-1-yl)methyl)phenoxy)methyl)nicotinamido)-5-guanidinopentanoate

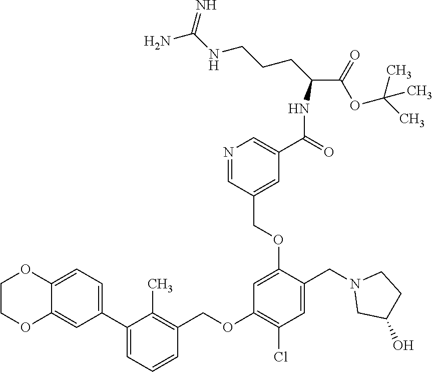 [SMILES: Cc1c(COc2cc(OCc3cncc(C(=O)N[C@@H](CCCNC(=N)N)C(=O)OC(C)(C)C)c3)c(CN3CC[C@H](O)C3)cc2Cl)cccc1-c1ccc2c(c1)OCCO2]

(S)-tert-Butyl 2-(5-((4-chloro-5-((3-(2,3-dihydrobenzo[b][1,4]dioxin-6-yl)-2-methylbenzyl)oxy)-2-(((S)-3-hydroxypyrrolidin-1-yl)methyl)phenoxy)methyl) nicotinamido)-5-guanidinopentanoate (crude) was obtained from (S)-5-((4-chloro-5-((3-(2,3-dihydrobenzo[b][1,4]dioxin-6-yl)-2-methylbenzyl)oxy)-2-((3-hydroxypyrrolidin-1-yl)methyl)phenoxy)methyl)nicotinic acid and H-Arg-OtBu·2HCl using the procedure for Example 2007.

### Example 2008: (S)-3-(5-((4-chloro-5-((3-(2,3-dihydrobenzo[b][1,4]dioxin-6-yl)-2-methylbenzyl)oxy)-2-((3-hydroxypyrrolidin-1-yl)methyl)phenoxy)methyl)nicotinamido)propanoic acid

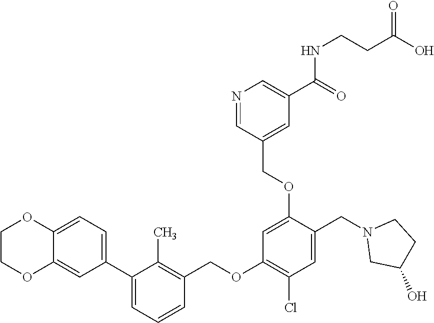 [SMILES: Cc1c(COc2cc(OCc3cncc(C(=O)NCCC(=O)O)c3)c(CN3CC[C@H](O)C3)cc2Cl)cccc1-c1ccc2c(c1)OCCO2]

(S)-3-(5-((4-Chloro-5-((3-(2,3-dihydrobenzo[b][1,4]dioxin-6-yl)-2-methylbenzyl)oxy)-2-((3-hydroxypyrrolidin-1-yl)methyl)phenoxy)methyl) nicotinamido)propanoic acid was obtained from (S)-tert-butyl 3-(5-((4-chloro-5-((3-(2,3-dihydrobenzo[b][1,4]dioxin-6-yl)-2-methylbenzyl)oxy)-2-((3-hydroxypyrrolidin-1-yl)methyl)phenoxy)methyl)nicotinamido)propanoate and lithium hydroxide using the procedure described for (S)-5-((4-chloro-5-((3-(2,3-dihydrobenzo[b][1,4]dioxin-6-yl)-2-methylbenzyl)oxy)-2-((3-hydroxypyrrolidin-1-yl)methyl)phenoxy)methyl)nicotinic acid. The crude material was purified via preparative LC/MS with the following conditions: Column: XBridge C18, 19×200 mm, 5-μm particles; Mobile Phase A: 5:95 methanol: water with 10-mM ammonium acetate; Mobile Phase B: 95:5 methanol: water with 10-mM ammonium acetate; Gradient: 40-80% B over 20 minutes, then a 5-minute hold at 100% B; Flow: 20 mL/min. Fractions containing the desired product were combined and dried via centrifugal evaporation. ¹H NMR (500 MHz, DMSO-d₆) δ 8.96 (s, 1H), 8.82 (s, 1H), 8.30 (s, 1H), 7.47 (d, J=7.3 Hz, 1H), 7.35 (s, 1H), 7.28-7.22 (m, 1H), 7.18 (d, J=7.7 Hz, 1H), 7.14 (s, 1H), 6.93 (d, J=8.1 Hz, 1H), 6.80-6.73 (m, 2H), 5.32 (s, 2H), 5.24 (s, 2H), 4.28 (s, 4H), 4.18 (br. s., 1H), 3.64-3.53 (m, 2H), 3.52-3.44 (m, 2H), 2.69 (dd, J=9.5, 6.2 Hz, 1H), 2.66-2.58 (m, 1H), 2.5-2.47 (m, 2H), 2.47-2.41 (m, 1H), 2.37 (dd, J=9.7, 3.1 Hz, 1H), 2.25 (s, 3H), 2.04-1.94 (m, 1H), 1.54 (dd, J=8.3, 5.0 Hz, 1H). Two analytical LC/MS injections were used to determine the final purity.

Injection 1 conditions: Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B, 0-100% B over 3 minutes, then a 0.5-minute hold at 100% B; Flow: 1 mL/min; Detection: UV at 220 nm.

Injection 2 conditions: Column: Waters BEH C18, 2.0×50 mm, 1.7-μm particles; Mobile Phase A: 5:95 methanol:water with 10 mM ammonium acetate; Mobile Phase B: 95:5 methanol:water with 10 mM ammonium acetate; Temperature: 50° C.; Gradient: 0% B, 0-100% B over 3 minutes, then a 0.5-minute hold at 100% B; Flow: 0.5 mL/min; Detection: UV at 220 nm.

LCMS (Injection 1 condition) Rt=1.55 min, m/z 688 (M+1).

LCMS (Injection 2 condition) Rt=2.62 min, m/z 688 (M+1).

### Example 2009: (S)-2-(5-((4-chloro-5-((3-(2,3-dihydrobenzo[b][1,4]dioxin-6-yl)-2-methylbenzyl)oxy)-2-(((S)-3-hydroxypyrrolidin-1-yl)methyl)phenoxy)methyl)nicotinamido)-3-hydroxypropanoic acid

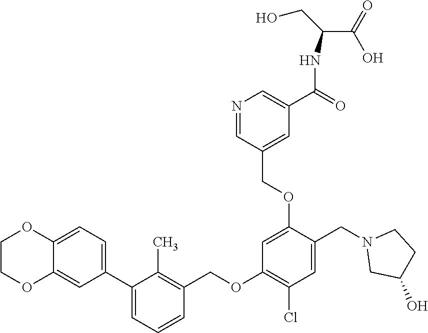 [SMILES: Cc1c(COc2cc(OCc3cncc(C(=O)N[C@@H](CO)C(=O)O)c3)c(CN3CC[C@H](O)C3)cc2Cl)cccc1-c1ccc2c(c1)OCCO2]

(S)-2-(5-((4-Chloro-5-((3-(2,3-dihydrobenzo[b][1,4]dioxin-6-yl)-2-methylbenzyl)oxy)-2-(((S)-3-hydroxypyrrolidin-1-yl)methyl)phenoxy) methyl)nicotinamido)-3-hydroxypropanoic acid was obtained from (S)-ethyl 2-(5-((4-chloro-5-((3-(2,3-dihydrobenzo[b][1,4]dioxin-6-yl)-2-methylbenzyl)oxy)-2-(((S)-3-hydroxypyrrolidin-1-yl)methyl)phenoxy)methyl)nicotinamido)-3-hydroxypropanoate and lithium hydroxide using the procedure described for (S)-5-((4-chloro-5-((3-(2,3-dihydrobenzo[b][1,4]dioxin-6-yl)-2-methylbenzyl)oxy)-2-((3-hydroxypyrrolidin-1-yl)methyl)phenoxy)methyl)nicotinic acid. The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-50% B over 30 minutes, then a 5-minute hold at 100% B; Flow: 20 mL/min. Fractions containing the desired product were combined and dried via centrifugal evaporation. ¹H NMR (500 MHz, DMSO-d₆) δ 8.99 (br. s., 1H), 8.83 (s, 1H), 8.37 (br. s., 1H), 7.48 (d, J=7.7 Hz, 1H), 7.37 (s, 1H), 7.26 (t, J=7.3 Hz, 1H), 7.21-7.13 (m, 2H), 6.93 (d, J=8.4 Hz, 1H), 6.82-6.74 (m, 2H), 5.33 (s, 2H), 5.26 (s, 2H), 4.29 (s, 4H), 4.34-4.24 (m, 1H), 4.19 (br. s., 1H), 3.77-3.57 (m, 4H), 2.79-2.64 (m, 2H), 2.54-2.46 (m, 1H), 2.43 (d, J=10.3 Hz, 1H), 2.26 (s, 3H), 2.05-1.93 (m, 1H), 1.56 (br. s., 1H).

Two analytical LC/MS injections were used to determine the final purity.

Injection 1 conditions: Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.5-minute hold at 100% B; Flow: 1.0 mL/min; Detection: UV at 220 nm.

Injection 2 conditions: Column: Waters BEH C18, 2.0×50 mm, 1.7-μm particles; Mobile Phase A: 5:95 methanol:water with 10 mM ammonium acetate; Mobile Phase B: 95:5 methanol:water with 10 mM ammonium acetate; Temperature: 50° C.; Gradient: 0-100% B over 3 minutes, then a 0.5-minute hold at 100% B; Flow: 0.5 mL/min; Detection: UV at 220 nm

LCMS (Injection 1 condition) Rt=1.555 min, m/z 704 (M+1), 702(M−1).

LCMS (Injection 2 condition) Rt=3.029 min, m/z 704 (M+1), 702 (M−1).

### Example 2010: (S)-2-(5-((4-chloro-5-((3-(2,3-dihydrobenzo[b][1,4]dioxin-6-yl)-2-methylbenzyl)oxy)-2-(((S)-3-hydroxypyrrolidin-1-yl)methyl)phenoxy)methyl)nicotinamido)-5-guanidinopentanoic acid

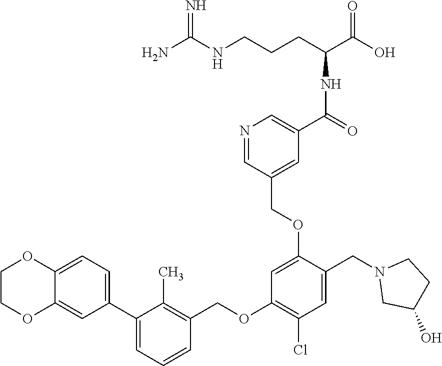 [SMILES: Cc1c(COc2cc(OCc3cncc(C(=O)N[C@@H](CCCNC(=N)N)C(=O)O)c3)c(CN3CC[C@H](O)C3)cc2Cl)cccc1-c1ccc2c(c1)OCCO2]

(S)-2-(5-((4-Chloro-5-((3-(2,3-dihydrobenzo[b][1,4]dioxin-6-yl)-2-methylbenzyl)oxy)-2-(((S)-3-hydroxypyrrolidin-1-yl)methyl)phenoxy)methyl) nicotinamido)-5-guanidinopentanoic acid was obtained from (S)-tert-butyl 2-(5-((4-chloro-5-((3-(2,3-dihydrobenzo[b][1,4]dioxin-6-yl)-2-methylbenzyl)oxy)-2-(((S)-3-hydroxypyrrolidin-1-yl)methyl)phenoxy)methyl)nicotinamido)-5-guanidinopentanoate and lithium hydroxide using the procedure described for (S)-5-((4-chloro-5-((3-(2,3-dihydrobenzo[b][1,4]dioxin-6-yl)-2-methylbenzyl)oxy)-2-((3-hydroxypyrrolidin-1-yl)methyl)phenoxy)methyl)nicotinic acid. The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-80% B over 30 minutes, then a 5-minute hold at 100% B; Flow: 20 mL/min. Fractions containing the desired product were combined and dried via centrifugal evaporation. ¹H NMR (500 MHz, DMSO-d₆) δ 8.87 (s, 1H), 8.64 (s, 1H), 8.14 (br. s., 1H), 7.46 (d, J=7.3 Hz, 1H), 7.30 (s, 1H), 7.24 (t, J=7.5 Hz, 1H), 7.17 (d, J=7.3 Hz, 1H), 7.09 (s, 1H), 6.92 (d, J=8.4 Hz, 1H), 6.81-6.72 (m, 2H), 5.25-5.11 (m, 4H), 4.32-4.21 (br, s, 1H), 4.28 (s, 4H), 4.15 (br. s., 1H), 3.59-3.45 (m, 2H), 3.11 (br. s., 1H), 3.02 (br. s., 1H), 2.65-2.53 (m, 2H), 2.34 (m, 2H), 2.23 (s, 3H), 2.00-1.92 (m, 2H), 1.85 (br. s., 1H), 1.65-1.45 (m, 3H).

Two analytical LC/MS injections were used to determine the final purity.

Injection 1 conditions: Column: Waters BEH C18, 2.0×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.5-minute hold at 100% B; Flow: 1.0 mL/min; Detection: UV at 220 nm.

Injection 2 conditions: Column: Waters BEH C18, 2.0×50 mm, 1.7-μm particles; Mobile Phase A: 5:95 methanol:water with 10 mM ammonium acetate; Mobile Phase B: 95:5 methanol:water with 10 mM ammonium acetate; Temperature: 50° C.; Gradient: 0-100% B over 3 minutes, then a 0.5-minute hold at 100% B; Flow: 0.5 mL/min; Detection: UV at 220 nm.

LCMS (Injection 1 condition) Rt=1.537 min, m/z 773 (M+1), 771 (M−1).

LCMS (Injection 2 condition) Rt=3.074 min, m/z 773 (M+1), 771 (M−1).

### Example 2011: (2S)-1-(5-Chloro-4-((3-(2,3-dihydrobenzo[b][1,4]dioxin-6-yl)-2-methylbenzyl)oxy)-2-(morpholin-2-ylmethoxy)benzyl)piperidine-2-carboxylic acid acetic acid salt

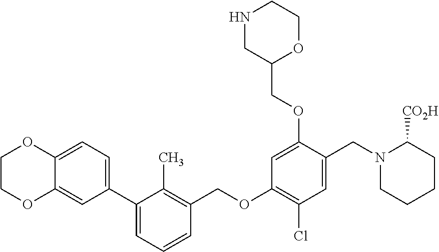 [SMILES: Cc1c(COc2cc(OCC3CNCCO3)c(CN3CCCC[C@H]3C(=O)O)cc2Cl)cccc1-c1ccc2c(c1)OCCO2]

Example 2011 was prepared in two steps in a similar fashion to examples above from 5-chloro-4-((3-(2,3-dihydrobenzo[b][1,4]dioxin-6-yl)-2-methylbenzyl)oxy)-2-hydroxybenzaldehyde, tert-butyl 2-(chloromethyl)morpholine-4-carboxylate, and (S)-piperidine-2-carboxylic acid. The third and final step, the Boc deprotection step, was accomplished as follows: The Boc-precursor was taken up in dry DCM (0.5 mL) and TFA (200 μL, 2.59 mmol) was added to the stirred mixture at rt. After 2 h, this mixture was concentrated with nitrogen stream and the residue was taken up in MeOH, filtered through a syringe filter and purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 22-62% B over 20 minutes, then a 4-minute hold at 100% B; Flow: 20 mL/min. Fractions containing the desired product were combined and dried via centrifugal evaporation. The material was further purified via preparative LC/MS with the following conditions: Column: XBridge C18, 19×200 mm, 5-μm particles; Mobile Phase A: 5:95 methanol: water with 10-mM ammonium acetate; Mobile Phase B: 95:5 methanol: water with 10-mM ammonium acetate; Gradient: 40-85% B over 25 minutes, then a 5-minute hold at 100% B; Flow: 20 mL/min. Fractions containing the desired product were combined and dried via centrifugal evaporation. The yield of the product was 3.4 mg, and its estimated purity by LCMS analysis was 96%. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.75 min hold at 100% B; Flow: 1 mL/min; Detection: MS and UV (220 nm). Injection 1 results: Purity: 95.8%; Observed Mass: 623.18; Retention Time: 1.86 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.75 min hold at 100% B; Flow: 1 mL/min; Detection: MS and UV (220 nm). Injection 2 results: Purity: 96.3%; Observed Mass: 623.21; Retention Time: 1.83 min. ¹H NMR (500 MHz, DMSO-d₆) δ 7.49 (d, J=7.7 Hz, 1H), 7.45 (s, 1H), 7.27 (t, J=7.5 Hz, 1H), 7.19 (d, J=7.3 Hz, 1H), 6.99 (s, 1H), 6.93 (d, J=8.1 Hz, 1H), 6.79 (d, J=2.2 Hz, 1H), 6.76 (dd, J=8.3, 2.0 Hz, 1H), 5.26 (s, 2H), 4.29 (s, 4H), 4.09-3.97 (m, 2H), 3.91 (s, 1H), 3.86-3.80 (m, 1H), 3.79-3.72 (m, 2H), 3.70-3.64 (m, 1H), 3.18-3.09 (m, 1H), 3.02-2.86 (m, 2H), 2.77-2.62 (m, 2H), 2.61-2.53 (m, 2H), 2.44-2.32 (m, 1H), 2.25 (s, 3H), 1.91 (s, 3H), 1.89-1.80 (m, 1H), 1.74-1.64 (m, 1H), 1.57-1.46 (m, 3H), 1.42-1.37 (m, 1H).

### Example 2012: (2S)-1-(5-Chloro-4-((3-(2,3-dihydrobenzo[b][1,4]dioxin-6-yl)-2-methyl benzyl)oxy)-2-((1-methylpiperidin-3-yl)methoxy)benzyl)piperidine-2-carboxylic acid

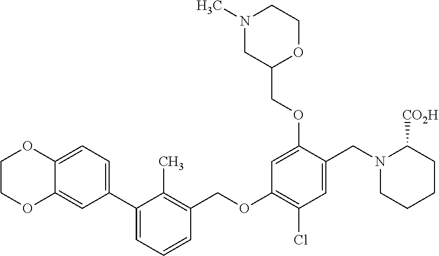 [SMILES: Cc1c(COc2cc(OCC3CN(C)CCO3)c(CN3CCCC[C@H]3C(=O)O)cc2Cl)cccc1-c1ccc2c(c1)OCCO2]

Example 2012 was prepared in two steps in a similar fashion to examples above from 5-chloro-4-((3-(2,3-dihydrobenzo[b][1,4]dioxin-6-yl)-2-methylbenzyl)oxy)-2-hydroxybenzaldehyde, 3-(chloromethyl)-1-methylpiperidine HCl, and (S)-piperidine-2-carboxylic acid. The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 26-66% B over 20 minutes, then a 4-minute hold at 100% B; Flow: 20 mL/min. Fractions containing the desired product were combined and dried via centrifugal evaporation. The yield of the product was 7.7 mg, and its estimated purity by LCMS analysis was 99%. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.75 min hold at 100% B; Flow: 1 mL/min; Detection: MS and UV (220 nm). Injection 1 results: Purity: 100.0%; Observed Mass: 635.27; Retention Time: 1.91 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.75 min hold at 100% B; Flow: 1 mL/min; Detection: MS and UV (220 nm). Injection 2 results: Purity: 98.7%; Observed Mass: 635.27; Retention Time: 1.88 min. ¹H NMR (500 MHz, DMSO-d₆) δ 7.49 (d, J=7.3 Hz, 1H), 7.42 (s, 1H), 7.27 (t, J=7.5 Hz, 1H), 7.19 (d, J=7.7 Hz, 1H), 6.96-6.90 (m, 2H), 6.78 (s, 1H), 6.77-6.74 (m, 1H), 5.26 (s, 2H), 4.29 (s, 4H), 3.98-3.87 (m, 2H), 3.79-3.72 (m, 1H), 3.64-3.58 (m, 1H), 3.18-3.08 (m, 1H), 2.98-2.88 (m, 1H), 2.84-2.78 (m, 1H), 2.68-2.60 (m, 1H), 2.35-2.28 (m, 1H), 2.25 (s, 3H), 2.17 (s, 3H), 2.07-1.98 (m, 1H), 1.97-1.92 (m, 1H), 1.88-1.78 (m, 2H), 1.75-1.69 (m, 2H), 1.68-1.61 (m, 1H), 1.55-1.46 (m, 4H), 1.42-1.32 (m, 1H), 1.18-1.03 (m, 1H).

### Biological Assays

The ability of the compounds of the invention to bind to PD-L1 was investigated using a PD-1/PD-L1 Homogenous Time-Resolved Fluorescence (HTRF) binding assay.

The interaction of PD-1 and PD-L1 can be assessed using soluble, purified preparations of the extracellular domains of the two proteins. The PD-1 and PD-L1 protein extracellular domains were expressed as fusion proteins with detection tags, for PD-1, the tag was the Fc portion of Immunoglobulin (PD-1-Ig) and for PD-L1 it was the 6 histidine motif (PD-L1-His). All binding studies were performed in an HTRF assay buffer consisting of dPBS supplemented with 0.1% (with) bovine serum albumin and 0.05% (v/v) Tween-20. For the h/PD-L1-His binding assay, inhibitors were pre-incubated with PD-L1-His (10 nM final) for 15m in 4 μl of assay buffer, followed by addition of PD-1-Ig (20 nM final) in 1 μl of assay buffer and further incubation for 15 m. HTRF detection was achieved using europium crypate-labeled anti-Ig (1 nM final) and allophycocyanin (APC) labeled anti-His (20 nM final). Antibodies were diluted in HTRF detection buffer and 5 μl was dispensed on top of the binding reaction. The reaction mixture was allowed to equilibrate for 30 minutes and the resulting signal (665 nm/620 nm ratio) was obtained using an EnVision fluorometer. Additional binding assays were established between the human proteins PD-1-Ig/PD-L2-His (20 & 5 nM, respectively) and CD80-His/PD-L1-Ig (100 & 10 nM, respectively).

Recombinant Proteins: Human PD-1 (25-167) with a C-terminal human Fc domain of immunoglobulin G (Ig) epitope tag [hPD-1 (25-167)-3S-IG] and human PD-L1 (18-239) with a C-terminal His epitope tag [hPD-L1(18-239)-TVMV-His] were expressed in HEK293T cells and purified sequentially by ProteinA affinity chromatography and size exclusion chromatography. Human PD-L2-His and CD80-His was obtained through commercial sources.

### Methods

Homogenous Time-Resolved Fluorescence (HTRF) Assays of Binding of Soluble PD-1 to Soluble PD-L1. Soluble PD-1 and soluble PD-L1 refers to proteins with carboxyl-end truncations that remove the transmembrane-spanning regions and are fused to heterologous sequences, specifically the Fc portion of the human immunoglobuling G sequence (Ig) or the hexahistidine epitope tag (His). All binding studies were performed in an HTRF assay buffer consisting of dPBS supplemented with 0.1% (w/v) bovine serum albumin and 0.05% (v/v) Tween-20. For the PD-1-Ig/PD-L1-His binding assay, inhibitors were pre-incubated with PD-L1-His (10 nM final) for 15m in 4 μl of assay buffer, followed by addition of PD-1-Ig (20 nM final) in 1 μl of assay buffer and further incubation for 15 m. PD-L1 fusion proteins from either human, cynomologous macaques, mouse, or other species were used. HTRF detection was achieved using europium crypate-labeled anti-Ig monoclonal antibody (1 nM final) and allophycocyanin (APC) labeled anti-His monoclonal antibody (20 nM final). Antibodies were diluted in HTRF detection buffer and 5 μl was dispensed on top of binding reaction. The reaction was allowed to equilibrate for 30 minutes and signal (665 nm/620 nm ratio) was obtained using an EnVision fluorometer. Additional binding assays were established between PD-1-Ig/PD-L2-His (20 and 5 nM, respectively), CD80-His/PD-L1-Ig (100 and 10 nM, respectively) and CD80-His/CTLA4-Ig (10 and 5 nM, respectively).

Binding/competition studies between biotinylated Compound No. 71 and human PD-L1-His were performed as follows. The compounds of the present invention were pre-incubated with PD-L1-His (10 nM final) for 60 minutes in 4 μl of assay buffer followed by addition of biotinylated Compound No. 71 (0.5 nM final) in 1 μl of assay buffer. Binding was allowed to equilibrate for 30 minutes followed by addition of europium crypated labeled Streptavidin (2.5 pM final) and APC-labeled anti-His (20 nM final) in 5 μl of HTRF buffer. The reaction was allowed to equilibrate for 30m and signal (665 nm/620 nm ratio) was obtained using an EnVision fluorometer.

Recombinant Proteins. Carboxyl-truncated human PD-1 (amino acids 25-167) with a C-terminal human Ig epitope tag [hPD-1 (25-167)-3S-IG] and human PD-L1 (amino acids 18-239) with a C-terminal His epitope tag [hPD-L1(19-239)-tobacco vein mottling virus protease cleavage site (TVMV)-His] were expressed in HEK293T cells and purified sequentially by recombinant Protein A affinity chromatography and size exclusion chromatography. Human PD-L2-His (Sino Biologicals), CD80-His (Sino Biologicals), CTLA4-Ig (RnD Systems) were all obtained through commercial sources.

The table below lists the IC₅₀ values for representative examples of this disclosure measured in the PD-1/PD-L1 Homogenous Time-Resolved Fluorescence (HTRF) binding assay.

| Example Number | HTRF IC₅₀ (μM) |
|---|---|
| 1001 | 0.0045 |
| 1002 | 0.0068 |
| 1003 | 0.0058 |
| 1004 | 0.0173 |
| 1005 | 0.0079 |
| 1006 | 0.0011 |
| 1007 | 0.0276 |
| 1008 | 0.0017 |
| 1009 | 0.0437 |
| 1010 | 0.0434 |
| 1011 | 0.0110 |
| 1012 | 0.0030 |
| 1013 | 0.0720 |
| 1014 | 0.0066 |
| 1015 | 0.0684 |
| 1016 | 0.0054 |
| 1017 | 0.0202 |
| 1018 | 0.0040 |
| 1019 | 0.0041 |
| 1020 | 0.0031 |
| 1021 | 0.0057 |
| 1022 | 0.0027 |
| 1023 | 0.0046 |
| 1024 | 0.0005 |
| 1025 | 0.0156 |
| 1026 | 0.0022 |
| 1027 | 0.0041 |
| 1028 | 0.0018 |
| 1029 | 0.0012 |
| 1030 | 0.0065 |
| 1031 | 0.0147 |
| 1032 | 0.0044 |
| 1033 | 0.0039 |
| 1034 | 0.0039 |
| 1035 | 0.0034 |
| 1036 | 0.0046 |
| 1037 | 0.0018 |
| 1038 | 0.0057 |
| 1039 | 0.0023 |
| 1040 | 0.1644 |
| 1041 | 0.0505 |
| 1042 | 0.0006 |
| 1043 | 0.0004 |
| 1044 | 0.0021 |
| 1045 | 0.0044 |
| 1046 | 0.0070 |
| 1047 | 0.0054 |
| 1048 | 0.0111 |
| 1049 | 0.0142 |
| 1050 | 0.0598 |
| 1051 | 0.0010 |
| 1052 | 0.0013 |
| 1053 | 0.0014 |
| 1054 | 0.0018 |
| 1055 | 0.0004 |
| 1056 | 0.0006 |
| 1057 | 0.0062 |
| 1058 | 0.0218 |
| 1059 | 0.0022 |
| 1060 | 0.0092 |
| 1061 | 0.4548 |
| 2001 | 0.0095 |
| 2002 | 0.0065 |
| 2003 | 0.0065 |
| 2004 | 0.0058 |
| 2005 | 0.0019 |
| 2006 | 0.0028 |
| 2007 | 0.0034 |
| 2008 | 0.0052 |
| 2009 | 0.0046 |
| 2010 | 0.0065 |
| 2011 | 0.0265 |
| 2012 | 0.0285 |

The compounds of the present invention tested possess activity as inhibitors of the PD-1/PD-L1 interaction, and therefore, may be used in the treatment of diseases or deficiencies associated with the PD-1/PD-L1 interaction. Via inhibition of the PD-1/PD-L1 interaction, the compounds of the present disclosure may be employed to treat infectious diseases such as HIV, septic shock, Hepatitis A, B, C, or D and cancer.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound of Formula (I): 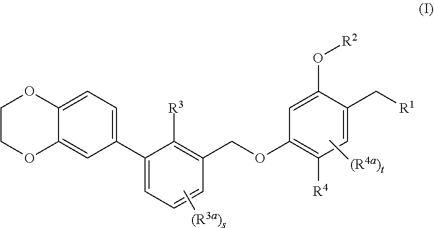
or a pharmaceutically acceptable salt thereof, wherein:
R¹ is independently 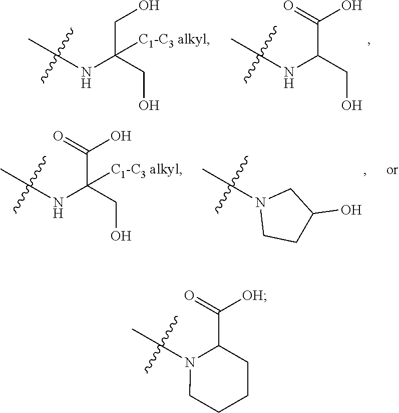
R² is independently —CH₂R^{2a}, —CH₂R^{2b}, or —(CH₂)ₘ(CHR^{a})ₙ—(CH₂)ₘR^{2b};
R^{2a} is independently C₄-C₆ cycloalkyl, 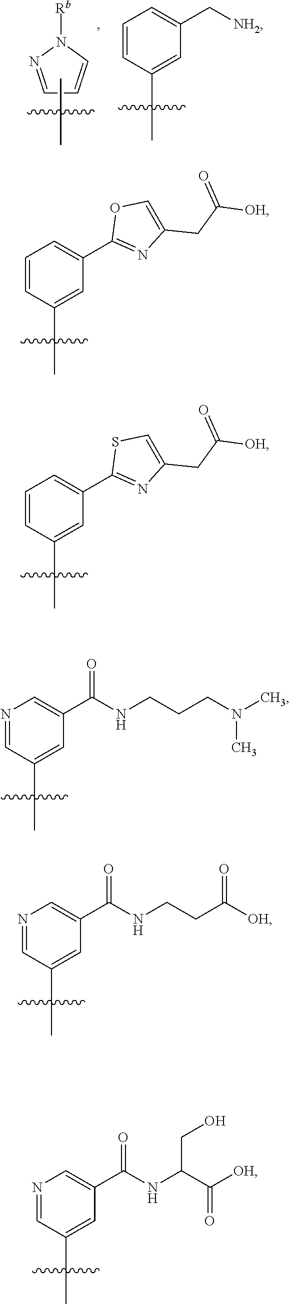 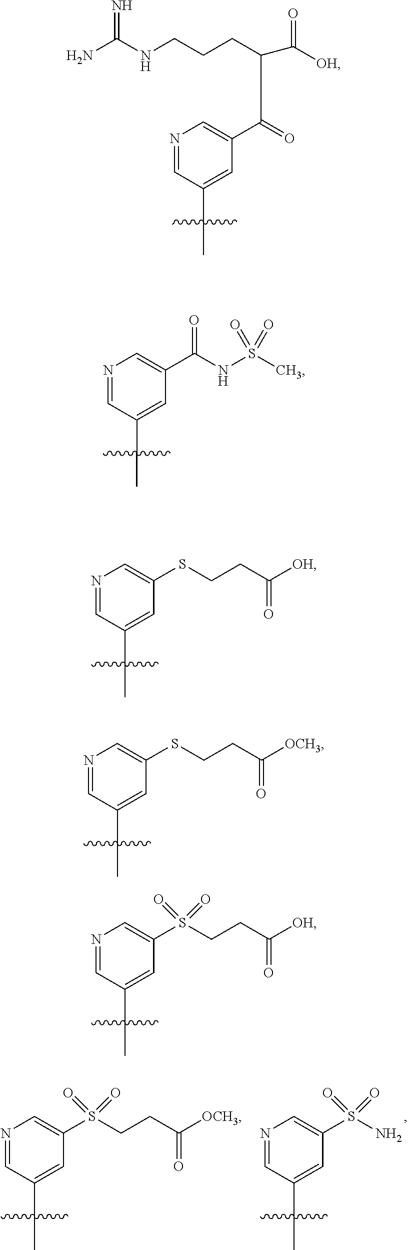 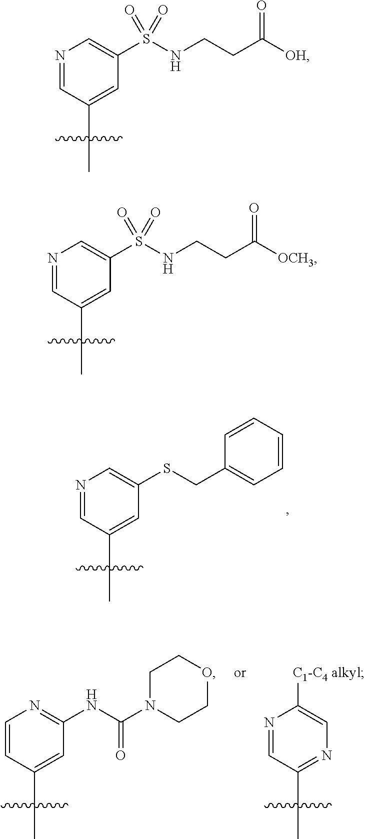
R^{2b} is independently OH, C₁-C₄ haloalkyl, —N(C₁-C₄ alkyl)₂, a 5- to 6-membered heterocyclic ring selected from pyrrolidinyl, tetrahydropyranyl, piperidinyl, morpholinyl 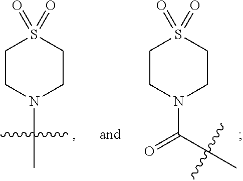 [SMILES: *N1CCS(=O)(=O)CC1 ; *C(=O)N1CCS(=O)(=O)CC1]
wherein said heterocyclic ring is substituted with 0 to 3 R^{c};
R³ and R⁴ are, at each occurrence, independently hydrogen, halogen, CN, C₁-C₄ alkyl, C₁-C₄haloalkyl, C₁-C₄ alkoxy, or C₁-C₄haloalkoxy;
R^{3a} and R^{4a} are, at each occurrence, independently halogen, CN, C₁-C₄ alkyl, C₁-C₄haloalkyl, C₁-C₄ alkoxy, or C₁-C₄haloalkoxy;
R^{a} is independently H or C₁-C₃ alkyl;
R^{b} is independently C₁-C₄ alkyl substituted with 0 to 1 R^{d}, or pyridyl;
R^{c} is independently OH, CN, halogen, C₁-C₄ alkyl, C₁-C₄haloalkyl, —C(═O)OH, or —C(═O)(OC₁-C₄ alkyl);
R^{d} is independently OH, CN, halogen, C₁-C₄haloalkyl, —C(═O)OH, or —C(═O)(OC₁-C₄ alkyl);
m is independently 1, 2 or 3;
n is independently 0 or 1;
s and t are each independently 0, 1, or 2.

2. A compound according to claim 1, wherein:
R³ and R⁴ are, at each occurrence, independently hydrogen, halogen, CN, C₁-C₄ alkyl or C₁-C₄haloalkyl;
R^{3a} and R^{4a} are, at each occurrence, independently halogen, CN, C₁-C₄ alkyl, or C₁-C₄haloalkyl;
R^{c} is independently OH, F, C₁, C₁-C₃ alkyl, C₁-C₂haloalkyl, —C(═O)OH, or —C(═O)(OC₁-C₂ alkyl);
R^{d} is independently CN, —C(═O)OH, or —C(═O)(OC₁-C₄ alkyl);
m is independently 1 or 2;
n is independently 0 or 1;
s and t are each independently 0 or 1.

3. A compound according to claim 1, wherein said compound is of Formula (II): 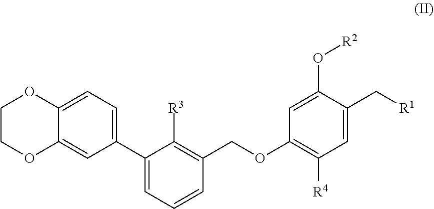
or a pharmaceutically acceptable salt thereof, wherein:
R¹ is independently 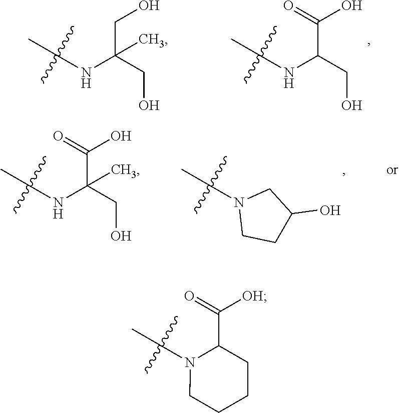
R² is independently —CH₂R^{2a}, —CH₂R^{2b}, or —(CH₂)ₘ(CHR^{a})ₙ—(CH₂)ₘR^{2b};
R^{a}is independently cyclobutyl, cyclohexyl, 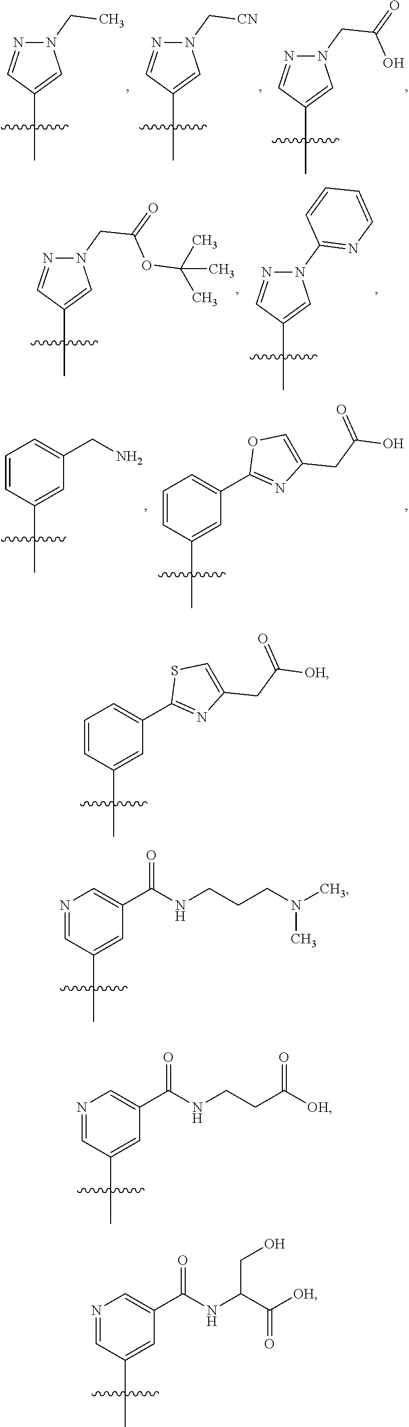 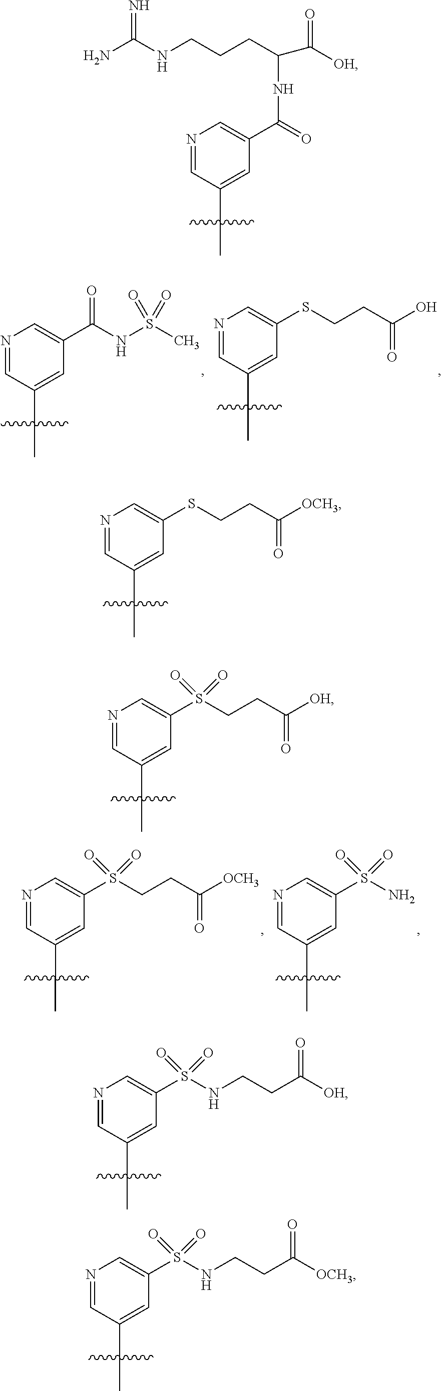 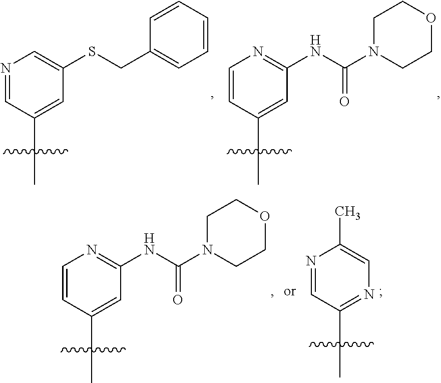
R^{2b} is independently OH, CF₃, —N(CH₃)₂, 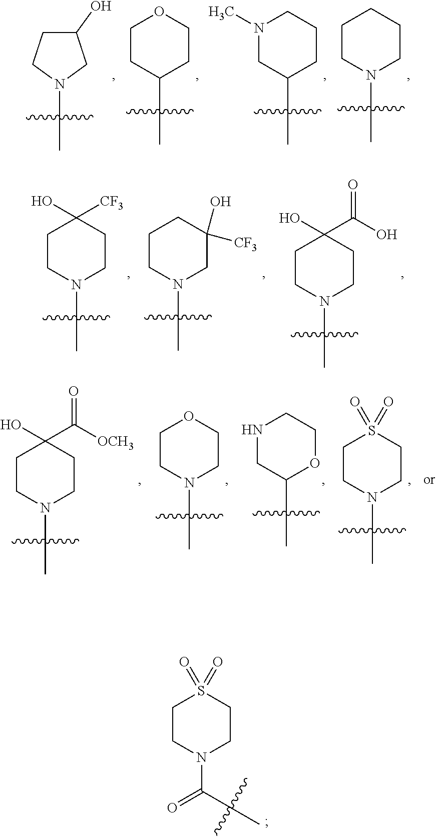
R³ is independently hydrogen, C₁-C₃ alkyl or halogen;
R⁴ is independently hydrogen, C₁-C₃ alkyl or halogen;
R^{a} is independently H or CH₃;
m is independently 1 or 2; and
n is independently 0 or 1.

4. A compound according to claim 3, wherein:
R² is independently —CH₂R^{2a};
R^{2a} is independently cyclobutyl, cyclohexyl, 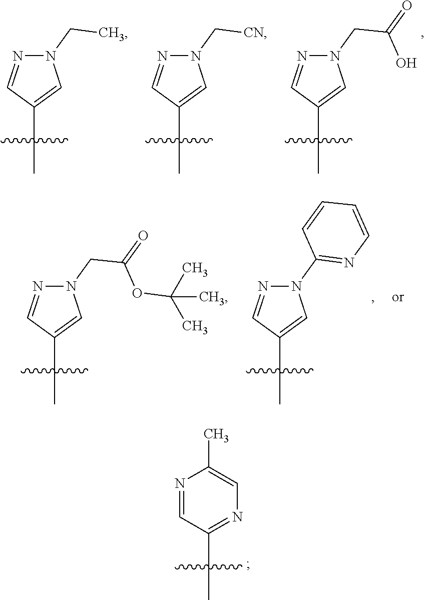
R³ is CH₃; and
R⁴ is Cl.

5. A compound according to claim 3, wherein:
R² is independently —CH₂R^{2a};
R^{2a} is independently 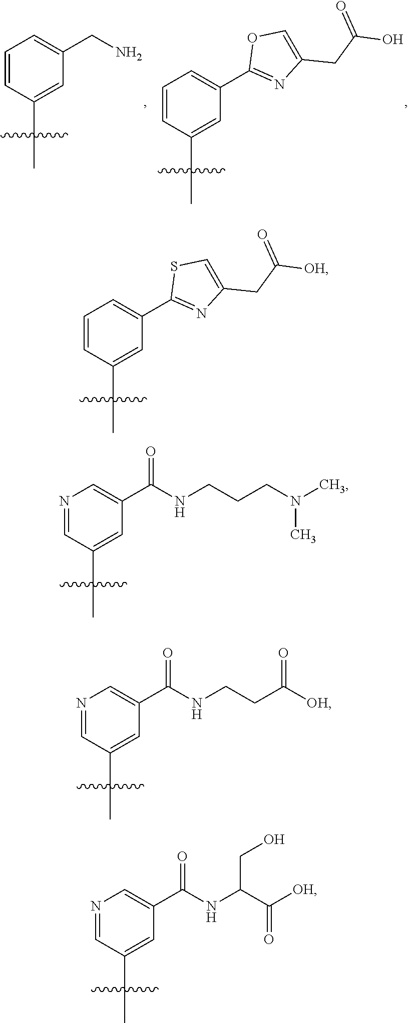 [SMILES: *c1cccc(CN)c1 ; *c1cccc(-c2nc(CC(=O)O)co2)c1 ; *c1cccc(-c2nc(CC(=O)O)cs2)c1 ; *c1cncc(C(=O)NCCCN(C)C)c1 ; *c1cncc(C(=O)NCCC(=O)O)c1 ; *c1cncc(C(=O)NC(CO)C(=O)O)c1] 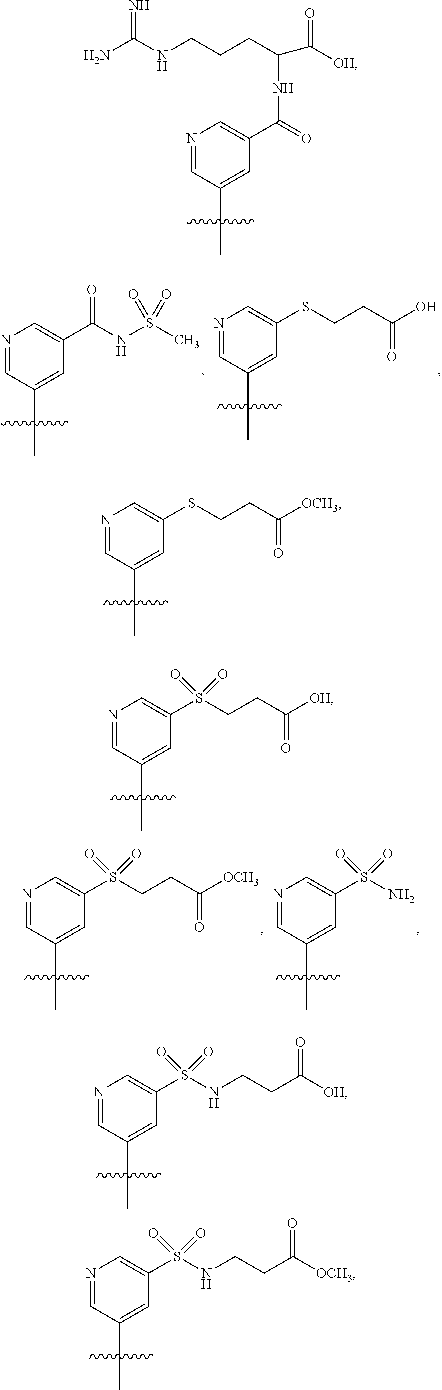 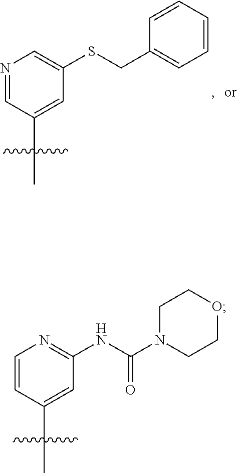 [SMILES: *c1cncc(SCc2ccccc2)c1 ; *c1ccnc(NC(=O)N2CCOCC2)c1]
R³ is CH₃; and
R⁴ is Cl.

6. A compound according to claim 3, wherein:
R² is independently —CH₂R^{2b}, or —(CH₂)ₘ(CHR^{a})˜—(CH₂)ₘR^{2b}.
R^{2b} is independently OH, CF₃, —N(CH₃)₂, 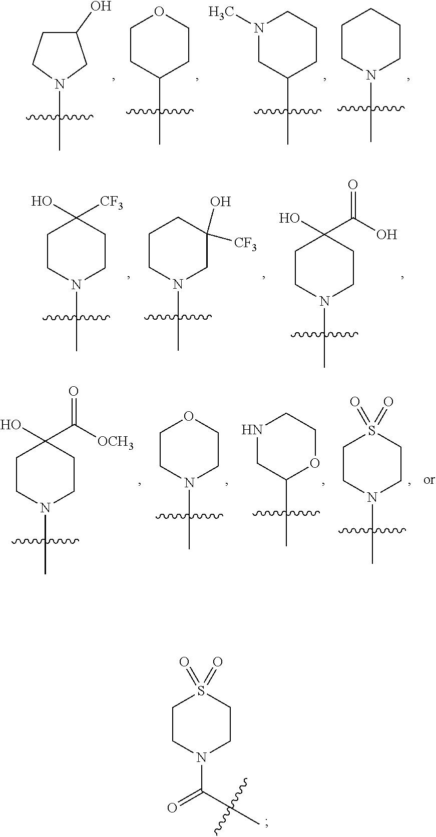
R³ is CH₃; and
R⁴ is Cl.

7. A compound of claim 1, wherein the compound is selected from the exemplified Examples or a pharmaceutically acceptable salt thereof.

8. A pharmaceutical composition comprising a compound of claim 1, or a pharmaceutically acceptable salt thereof, and a pharmaceutically acceptable carrier.

9. (canceled)

10. A compound or a pharmaceutically acceptable salt thereof according to claim 1, or a pharmaceutical composition according to claim 8, for use in the manufacture of a medicament for treatment of cancer in a subject in need thereof.

11. A compound or a pharmaceutically acceptable salt thereof according to claim 1, for use in enhancing, stimulating, modulating and/or increasing an immune response in a subject in need thereof, comprising administering to the subject a therapeutically effective amount of said compound or a pharmaceutically acceptable salt thereof.

12. A compound or a pharmaceutically acceptable salt thereof according to claim 1, for use in inhibiting growth, proliferation, or metastasis of cancer cells in a subject in need thereof, comprising administering to the subject a therapeutically effective amount of said compound or a pharmaceutically acceptable salt thereof.
